(19) United States
(12) Patent Application Publication
(10) Pub. No.: US 2024/0009136 A1
(43) Pub. Date: Jan. 11, 2024

(54) SYSTEM, METHOD, DEVICE, AND COMPOUND FOR FACILITATING MULTICOMPARTMENT CAPSULES

(71) Applicant: Multiply Labs Inc., San Francisco, CA (US)

(72) Inventors: Federico PARIETTI, San Francisco, CA (US); Enrico Giuseppe FERRO, San Francisco, CA (US); Alice MELOCCHI, Dalmine (IT); Bo ZHOU, San Francisco, CA (US)

(21) Appl. No.: 18/349,070

(22) Filed: Jul. 7, 2023

Related U.S. Application Data
(60) Provisional application No. 63/359,464, filed on Jul. 8, 2022.

(51) Int. Cl. A61K 9/48

(52) U.S. Cl. CPC A61K 9/4808

(57) ABSTRACT

Systems, methods, devices, and compounds for facilitating a capsule are provided. The capsule includes a first hollow body and a second hollow body configured to couple together, which collectively form an interior volume of the capsule. The capsule includes a divider that includes a base and a sidewall protruding from a first circumferential edge portion of the base. The sidewall includes an inner surface, an outer surface, and a second circumferential edge portion. The second circumferential edge portion is configured to continuously engage a unique circumferential surface of the capsule, which secures the divider within the interior volume of the capsule and forms at least two unique interior volumes based on a position of the t divider and the unique circumferential surface of the capsule. The capsule includes a plurality of pharmaceutical compositions, each of which occupies a unique interior volume in the at least two unique interior volumes.

### CROSS-REFERENCE TO RELATED APPLICATION

The present application claims priority to U.S. Provisional Patent Application No. 63/359,464, entitled “System, Method, Device, and Compound for Facilitating Multicompartment Capsules,” filed Jul. 8, 2022, which is hereby incorporated by reference in its entirety for all purposes.

### TECHNICAL FIELD

The present invention is directed to systems, methods, devices, and compounds for facilitating a multicompartment capsule.

### BACKGROUND

Oral administration is one of the most prevalent methods for delivering active ingredients or medicaments to the body. Conventionally, active ingredients or medicaments are orally administered in a variety of physical states such as solid, liquid, or powder. Capsules have become the preferred drug delivery systems (DDS) for administering oral dosages.

Conventional capsules include a first compartment section, known as a base, and a second compartment section referred to as a cap. The two compartments of the capsule are designed so that a material to be encapsulated is dispensed into the base, and the open end of the cap is correspondingly disposed over the open end of the base. The walls of the cap and base are in physical contact with one another, which forms a single internal compartment. A means for structurally sealing the cap to the base is also incorporated into the manufacturing of the capsule, which prevents contamination of the internal compartment of the capsule.

Moreover, advances in pharmacological therapy are achieved through the discovery of new molecules or the identification of more efficient methods of administration, e.g., the development of DDS. DDS exploits technological features, including design, composition, and manufacturing processes, to determine, modulate, and improve the drug availability at the site of action. In addition to the therapeutic advantages, DDS affords improvements in bioavailability, efficacy, and compliance, as well as overall drug dose and side effect reduction. As such, the economic and health benefits related to the reduction and control of development costs and line extension through these improvements are important to the success of this technology.

Furthermore, combination drug therapies within a single DDS have been rapidly gaining importance and prevalence in treating one or more health conditions exhibited by subjects. However, conventional solutions that provide combination drug therapies in a convenient form for consumption by a subject have proven difficult to realize. For instance, different drugs combined in the same dosage form are not always stable during manufacture and/or storage, and can physically and/or chemically interact with each other, or with one or more excipients, to produce degradation products. Moreover, when combined Each drug in the combination dosage form should show the desired release rate from the dosage form to get absorbed in sufficient quantities upon oral administration or release the drug to surrounding environment in case of other delivery routes.

Given the importance of DDS, new systems, methods, devices, and compounds for supporting DDS are needed in the art.

Given the above background, what is needed in the art are improved systems, methods, devices, and compounds for facilitating multicompartment capsules.

### SUMMARY

The present disclosure addresses the shortcomings disclosed above by providing systems, methods, devices, and compounds that facilitate realization of a multicompartment capsule.

In some embodiments, the invention provides a capsule. The capsule includes a first hollow body and a second hollow body. The first hollow body and the second hollow body are configured to couple together, which collectively form an interior volume of the capsule. Furthermore, each hollow body includes a closed end portion. Moreover, the capsule includes a first divider. The first divider includes a base and a sidewall protruding from a first circumferential edge portion of the base. The sidewall of the first divider includes an inner surface, an outer surface, and a second circumferential edge portion. Moreover, the second circumferential edge portion is configured to continuously engage a unique circumferential surface of the capsule. From this, the first divider is secured within the interior volume of the capsule based on a position of the first divider and the unique circumferential surface of the capsule. Moreover, this structure forms at least two unique interior volumes of the interior volume of the capsule based on the position of the first divider and the unique circumferential surface of the capsule. Additionally, the capsule includes a plurality of pharmaceutical compositions. Each respective pharmaceutical composition in the plurality of pharmaceutical compositions occupies a unique interior volume of the at least two unique interior volumes, (e.g., a first pharmaceutical composition is disposed in a first unique interior volume of the at least two unique interior volumes, a second pharmaceutical composition is disposed in a second unique interior volume of the at least two unique interior volumes, etc.). In some embodiments, each respective unique interior volume has a corresponding capacity (e.g., volume) that is measured as a fraction, or partition, of the interior volume of the capsule. Said otherwise, in some such embodiments, when the capsule includes the first divider, the first divider partitions the interior volume of the capsule to form the at least two unique interior volumes that includes the first unique interior volume and the second unique interior volume. In various embodiments, the first and second pharmaceutical compositions are the same composition. In various embodiments, the first and second pharmaceutical composition are different compositions. In an exemplary embodiment, the first unique interior volume is greater than the second unique interior volume and a greater amount of the first pharmaceutical composition is disposed therein compared to the amount of the second pharmaceutical composition disposed in the second unique interior volume. In an exemplary embodiment, the first unique interior volume is smaller than the second unique interior volume and a smaller amount of the first pharmaceutical composition is disposed therein compared to the amount of the second pharmaceutical composition disposed in the second unique interior volume. Accordingly, in some embodiments, by changing a size of the first divider and/or the position of the first divider within the capsule, and thus the partition of the interior volume of the capsule into the first and second unique interior volumes, the amount of each respective pharmaceutical composition in the plurality of pharmaceutical compositions is configurable.

In some embodiments, the first divider has a frustum shape.

In some embodiments, the capsule further includes a line of symmetry about a longitudinal axis of the capsule.

In some embodiments, the interior volume of the capsule is in a range of from about 0.1 milliliters (mL) to about 1.5 mL.

In some embodiments, the second circumferential edge portion deforms radially.

In some embodiments, the inner surface and/or the outer surface of the sidewall is generally concave or generally convex.

In some embodiments, the base further includes an upper surface and a lower surface. Moreover, in some embodiments, the upper surface and/or the lower surface of the base is generally concave or generally convex. In some embodiments, the upper surface and the lower surface of the base are parallel or substantially parallel.

In some embodiments, the capsule further includes a layer of a material disposed on a portion of the upper surface. The material is configured to prevent the upper surface from adhering to a different surface or face other than the layer of the material so disposed.

In some embodiments, the material includes a coefficient of friction in a range of from 0.1 to 0.8.

In some embodiments, the second circumferential edge portion includes a chamfer or rounded edge.

In some embodiments, an outer diameter of the second circumferential edge portion exclusively engages the unique circumferential surface of the capsule.

In some embodiments, the base of each divider further includes a third circumferential edge portion.

In some embodiments, the second circumferential edge portion includes a first diameter, and the third circumferential edge portion includes a second diameter different from the first diameter.

In some embodiments, the first hollow body or the second hollow body includes a third diameter greater than or equal to the second diameter.

In some embodiments, the third diameter is less than or equal to the first diameter.

In some embodiments, the third circumferential edge portion includes a chamfer or rounded edge.

In some embodiments, a draft angle of the sidewall is greater than zero.

In some embodiments, the draft angle is in a range of from about 0.1 degrees (°) to about 5°.

In some embodiments, a characteristic length of the sidewall is in a range of from about 0.5 millimeters (mm) to about 20 mm.

In some embodiments, the first hollow body, the second hollow body, the first, or a combination thereof is transparent, translucent, or opaque.

In some embodiments, the first divider is a monolithic body.

In some embodiments, the plurality of pharmaceutical compositions includes one or more liquid formulations, one or more solid formulations, one or more powdered formulations, one or more granule formulations, one or more gel formulations, or a combination thereof.

In some embodiments, the first divider is defined by a first orientation. Moreover, the capsule further includes a second divider defined by a second orientation.

In some embodiments, the first orientation is different from the second orientation.

In some embodiments, the first divider, the first hollow body, the second hollow body, or a combination thereof includes a material selected from the group consisting of: ethyl cellulose, hydroxypropyl cellulose, hydroxypropyl methyl cellulose, hydroxypropyl methyl cellulose acetate succinate, various types of methacrylic acid copolymers, polyethylene oxide, polyvinyl alcohol, polyvinyl alcohol-polyethylene glycol graft copolymer, and polyvinyl caprolactam-polyvinyl acetate-polyethylene glycol graft co-polymer.

In some embodiments, the first divider, the first hollow body, the second hollow body, or a combination thereof includes an absorbable material, a biodegradable material, or a combination thereof.

In some embodiments, the first divider, the first hollow body, the second hollow body, or a combination thereof includes a water soluble polymer, an erodible polymer, a degradable polymer, a swellable polymer, or a combination thereof.

In some embodiments, the water soluble polymer is sparingly soluble, slightly soluble, very slightly soluble, or practically insoluble.

In some embodiments, the invention is directed to providing a computer system for fabricating a capsule.

In some embodiments, the invention is directed to providing a computer readable storage medium. The computer readable storage medium stores one or more programs. The one or more programs includes instructions, which when executed by an electronic device with one or more processors and a memory, cause the electronic device to execute a method for fabricating a capsule.

In some embodiments, the invention is directed to providing a method for fabricating a capsule.

In some embodiments, the invention is directed to providing a device for fabricating a capsule.

In various embodiments, the capsule is additively manufactured and filled robotically via one or more robotic device under computer control.

In various embodiments, the invention provides a method of devising a single unit dosage formulation of a pharmaceutical composition for a selected subject to prevent, treat or ameliorate a selected condition of the subject. The method includes determining an appropriate dosages of two or more agents of use to prevent, treat or ameliorate the condition taking into consideration the release rate of a first pharmaceutical composition in a first unique interior volume, and the size of this volume. Moreover, the method includes taking into consideration the release rate of a second pharmaceutical composition in a second unique interior volume, and the size of this volume. Furthermore, the method includes determining an effective dosage of each of the first and second pharmaceutical compositions.

In some embodiments, the invention is directed to providing computer system. The computer system includes one or more processing units and a memory coupled to at least one processing unit of the one or more processing units. The memory stores at least one program for execution by the at least one processing unit, the at least one program including one or more instructions. For each respective subject in a plurality of subjects, in which the plurality of subjects includes more than 100 subjects, the one or more instructions includes receiving a respective first alert, in electronic form at the computer system, from a respective client device associated with a respective subject that the respective subject has initiated a corresponding method. The corresponding method includes determining appropriate dosages of two or more agents of use to prevent, treat or ameliorate the condition taking into consideration the release rate of a first pharmaceutical composition in a first unique interior volume, and the size of this volume. The corresponding method further includes taking into consideration the release rate of a second pharmaceutical composition in a second unique interior volume, and the size of this volume. Moreover, the corresponding method includes determining an effective dosage of each of the first and second pharmaceutical composition.

In an exemplary embodiment, the invention provides a method of preventing, treating or ameliorating a condition in a patient by administering to a subject in need thereof a capsule of the invention containing at least a first and a second pharmaceutical composition known to be of use in preventing, treating or ameliorating in an appropriate dosage of each pharmaceutical composition for preventing, treating or ameliorating a condition in the subject to whom the capsule is administered.

In some embodiments, the capsule of the invention is administered in a single (e.g., daily) dosage form. In some embodiments, a release profile of the capsule is an immediate release (e.g., a body of the capsule is readily dissolvable). Accordingly, in some such embodiments, the first pharmaceutical composition and the second pharmaceutical composition are released at the same time in a system of the subject, such as a digestive system of the subject when the capsule is swallowed. In some embodiments, the release profile of the capsule is a conditioned release, such as an extended release that has a release profile occurring over a first period of time greater than a second period of time of the release profile of the immediate release.

In some embodiments, the invention provides a method of fulfilling an order for a capsule of the present disclosure.

In some embodiments, the invention is directed to providing computer system. The computer system includes one or more processing units and a memory coupled to at least one processing unit of the one or more processing units. The memory stores at least one program for execution by the at least one processing unit, the at least one program including one or more instructions. For each respective subject in a plurality of subjects, in which the plurality of subjects includes more than 100 subjects, the one or more instructions includes receiving a respective first alert, in electronic form at the computer system, from a respective client device associated with a respective subject that the respective subject has initiated a corresponding method. The corresponding method includes fulfilling an order for a capsule according to the present disclosure.

### BRIEF DESCRIPTION OF THE DRAWINGS

FIG. 1 illustrates a block diagram illustrating an embodiment of a system for manufacture of a multicompartment capsule, in which, in which dashed boxes and lines indicate optional features, in accordance with an embodiment of the present disclosure;

FIG. 2 illustrates a plurality of components of a multicompartment capsule including a first hollow body, a second hollow body, and a plurality of dividers, in accordance with an embodiment of the present disclosure;

FIG. 3 illustrates a cross-sectional view of a multicompartment capsule including a first hollow body, a second hollow body, and a plurality of dividers, in accordance with an embodiment of the present disclosure;

FIG. 4 illustrates a perspective view of a first divider, in accordance with an embodiment of the present disclosure;

FIG. 5 illustrates a side view of a first divider, in accordance with an embodiment of the present disclosure;

FIG. 6 illustrates a cross-sectional view taken along lines A-A of FIG. 5;

FIG. 7 illustrates a top view of a first divider, in accordance with an embodiment of the present disclosure;

FIG. 8 illustrates a capsule including a first hollow body, a second hollow body, a plurality of dividers, and a plurality of pharmaceutical compositions, in accordance with an embodiment of the present disclosure;

FIG. 9 illustrates another capsule including a first hollow body, a second hollow body, a plurality of dividers, and a plurality of pharmaceutical compositions, in accordance with an embodiment of the present disclosure;

FIG. 10 illustrates a chart of a respective release profile of a conventional tablet including an amlodipine pharmaceutical composition and a second pharmaceutical composition, a capsule of the present disclosure including the amlodipine pharmaceutical composition and the second pharmaceutical composition, and a conventional capsule including the amlodipine pharmaceutical composition and the second pharmaceutical composition, in accordance with an embodiment of the present disclosure; and

FIG. 11 illustrates a time lapse of a manufacture of a capsule including a first hollow body, a second hollow body, a plurality of dividers, and a plurality of pharmaceutical compositions, in accordance with an embodiment of the present disclosure.

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

The present disclosure is directed to providing systems, methods, devices, and compounds for facilitating a capsule, more particularly, a multicompartment capsule. The capsule includes a first hollow body and a second hollow body. The first hollow body and the second hollow body are configured to couple together, which collectively form a shape having an interior volume of the capsule. Accordingly, each hollow body includes a closed end portion. The capsule includes a first divider. In some embodiments, the first divider includes a base and a sidewall protruding from a first circumferential edge portion of the base. The sidewall includes an inner surface, an outer surface, and a second circumferential edge portion. The second circumferential edge portion is configured to continuously engage a unique circumferential surface of the capsule. By continuously engaging the unique circumferential surface of the capsule, the first divider is secured at a unique position within the interior volume of the capsule based on a position of the first divider and the unique circumferential surface of the capsule. Moreover, by continuously engaging the unique circumferential surface of the capsule, the first divider forms at least two unique interior volumes of the interior volume of the capsule based on the position of the first divider and the unique circumferential surface of the capsule. Furthermore, the capsule includes a plurality of pharmaceutical compositions. Each respective pharmaceutical composition occupies a unique interior volume of the at least two unique interior volumes of the interior volume of the capsule. As a non-limiting example, a first pharmaceutical composition occupies a first unique interior volume of the at least two unique interior volumes, and a second pharmaceutical composition occupies a second unique interior volume of the at least two unique interior volumes of the capsule. Accordingly, the first divider provides separation of different pharmaceutical compositions and their different physical forms by forming a physical barrier within the interior volume of the capsule. From this structure, the first divider enhances a stability of the plurality of pharmaceutical compositions occupying the capsule. Furthermore, the first divider ensures no physical or chemical interaction between the plurality of pharmaceutical compositions, which ensures delivery of different agents of a respective pharmaceutical composition in their original, efficacious form. Accordingly, this structure of the first divider and the capsule decreases the cost of manufacture of the dosage form as no interaction studies will need to be conducted. Furthermore, in some embodiments, due to an electability of the first divider, the first divider continuously engages the unique circumferential surface of the capsule when the sidewall of the capsule is deformed, which prevents physical or chemical interaction between the plurality of pharmaceutical compositions. In this way, the first divider of the present disclosure allows for a multicompartment capsule that is cheap, efficient, and easy to manufacture drug delivery system for combined therapies (e.g., a plurality of pharmaceutical compositions). Combined therapies are essential in many world threatening diseases such as cardiovascular diseases. Therefore, the capsule of the present disclosure provides these combined therapies efficiently, without interaction, quickly without long period development and complex dosage forms.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, and components have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For instance, a first pharmaceutical composition could be termed a second pharmaceutical composition, and, similarly, a second pharmaceutical composition could be termed a first pharmaceutical composition, without departing from the scope of the present disclosure. The first pharmaceutical composition and the second pharmaceutical composition are both pharmaceutical compositions, but they are not the same pharmaceutical composition.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The foregoing description included example systems, methods, techniques, instruction sequences, and computing machine program products that embody illustrative implementations. For purposes of explanation, numerous specific details are set forth in order to provide an understanding of various implementations of the inventive subject matter. It will be evident, however, to those skilled in the art that implementations of the inventive subject matter may be practiced without these specific details. In general, well-known instruction instances, protocols, structures, and techniques have not been shown in detail.

The foregoing description, for purpose of explanation, has been described with reference to specific implementations. However, the illustrative discussions below are not intended to be exhaustive or to limit the implementations to the precise forms disclosed. Many modifications and variations are possible in view of the above teachings. The implementations are chosen and described in order to best explain the principles and their practical applications, to thereby enable others skilled in the art to best utilize the implementations and various implementations with various modifications as are suited to the particular use contemplated.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

For convenience in explanation and accurate definition in the appended claims, the terms “upper,” “lower,” “up,” “down,” “upwards,” “downwards,” “inner,” “outer,” “inside,” “outside,” “inwardly,” “outwardly,” “interior,” “exterior,” “front,” “rear,” “back,” “forwards,” and “backwards,” are used to describe features of the exemplary embodiments with reference to the positions of such features as displayed in the figures.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, +5%, or +1% of a given value. Where particular values are described in the application and claims, unless otherwise stated, the term “about” means within an acceptable error range for the particular value. The term “about” can have the meaning as commonly understood by one of ordinary skill in the art. The term “about” can refer to ±10%. The term “about” can refer to ±5%.

By “biodegradable,” as used herein, is meant materials that are bioresorbable and/or degrade and/or break down by mechanical degradation (e.g., dissolve, resorb, etc.) upon interaction with a physiological environment into components that are metabolizable or excretable, over a period of time from minutes to three years, preferably less than one year, while maintaining the requisite structural integrity.

By “absorbable,” as used herein, is meant materials that are bioresorbable and/or degrade and/or break down metabolically, an attribute of a host regardless of whether a structure degrades, corrodes, hydrolyzes, or dissolves.

As used herein, the term “pharmaceutical compound” refers to any physical state of a material. For instance, in some embodiments, the pharmaceutical compounds include but are not limited to one or more solids, one or more powders, one or more granules, one or more tablets, one or more liquids, one or more gel formulations, or a combination thereof.

Additionally, the terms “capsule” and “multicompartment capsule” are interchangeably used herein unless expressly stated otherwise.

Furthermore, when a reference number is given an “i^{th}” denotation, the reference number refers to a generic component, set, or embodiment. For instance, a pharmaceutical composition termed “pharmaceutical composition i” refers to the i^{th} pharmaceutical composition in a plurality of pharmaceutical compositions (e.g., a pharmaceutical composition 142-i in a plurality of pharmaceutical compositions 142).

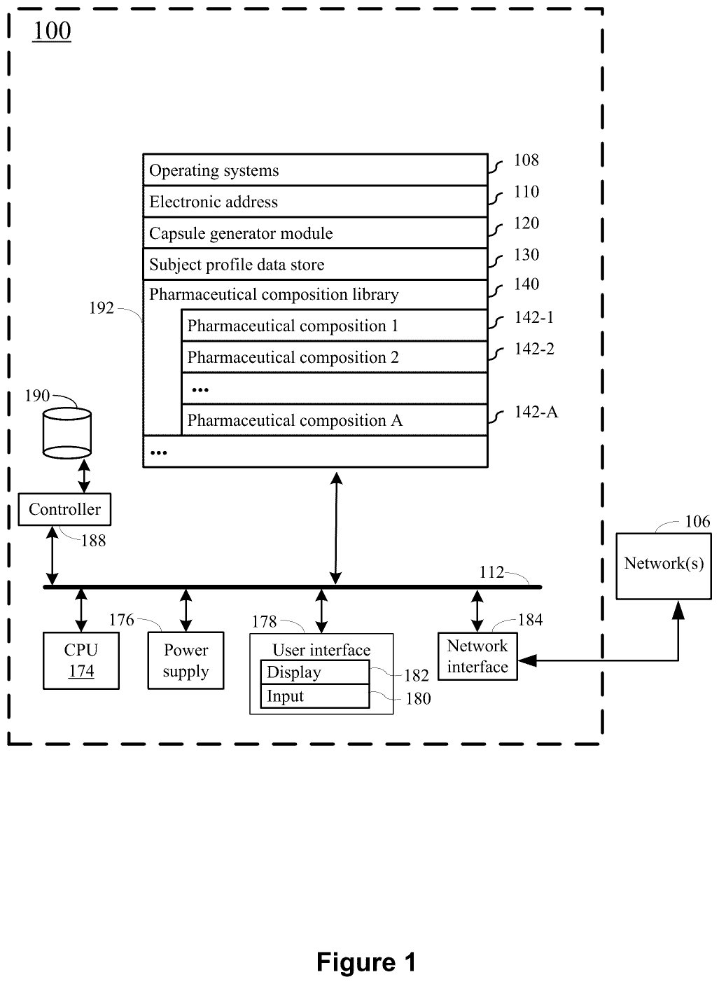

In the present disclosure, unless expressly stated otherwise, descriptions of devices and systems will include implementations of one or more computers. For instance, and for purposes of illustration in FIG. 1, a computer system (e.g., computer system 100 of FIG. 1) is represented as single device that includes all the functionality of the computer system. However, the present disclosure is not limited thereto. For instance, in some embodiments, the functionality of computer system 100 is spread across any number of networked computers and/or reside on each of several networked computers and/or by hosted on one or more virtual machines and/or containers at a remote location accessible across a communications network (e.g., communications network 106 of FIG. 1). One of skill in the art will appreciate that a wide array of different computer topologies is possible for computer system 100, and other devices and systems of the preset disclosure, and that all such topologies are within the scope of the present disclosure. Moreover, rather than relying on a physical communications network (e.g., communications network 106 of FIG. 1), the illustrated devices and systems may wirelessly transmit information between each other. As such, the exemplary topology shown in FIG. 1 merely serves to describe the features of an embodiment of the present disclosure in a manner that will be readily understood to one of skill in the art.

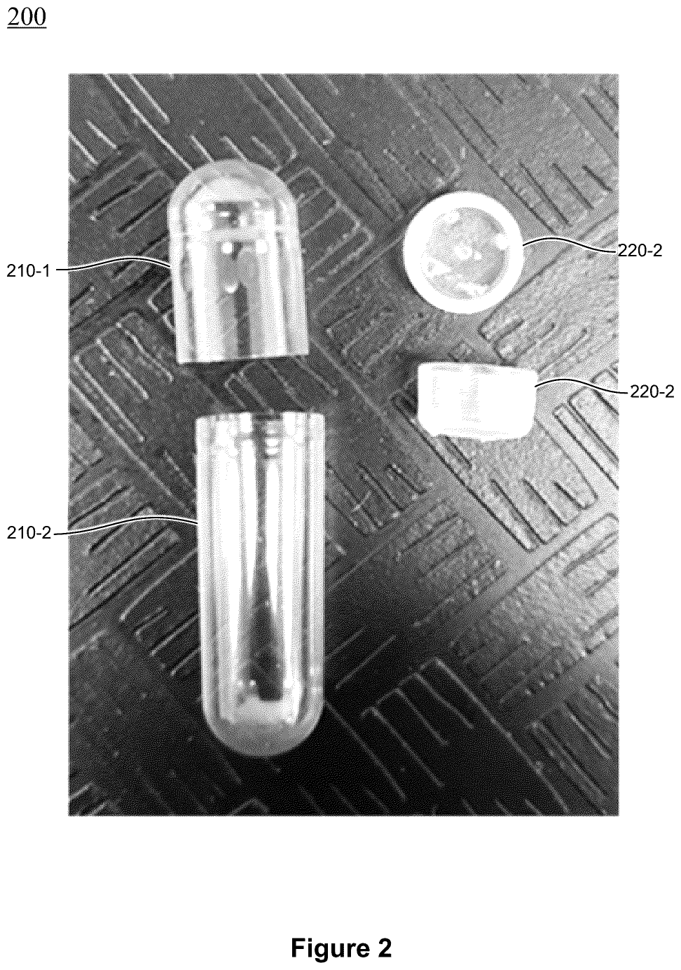

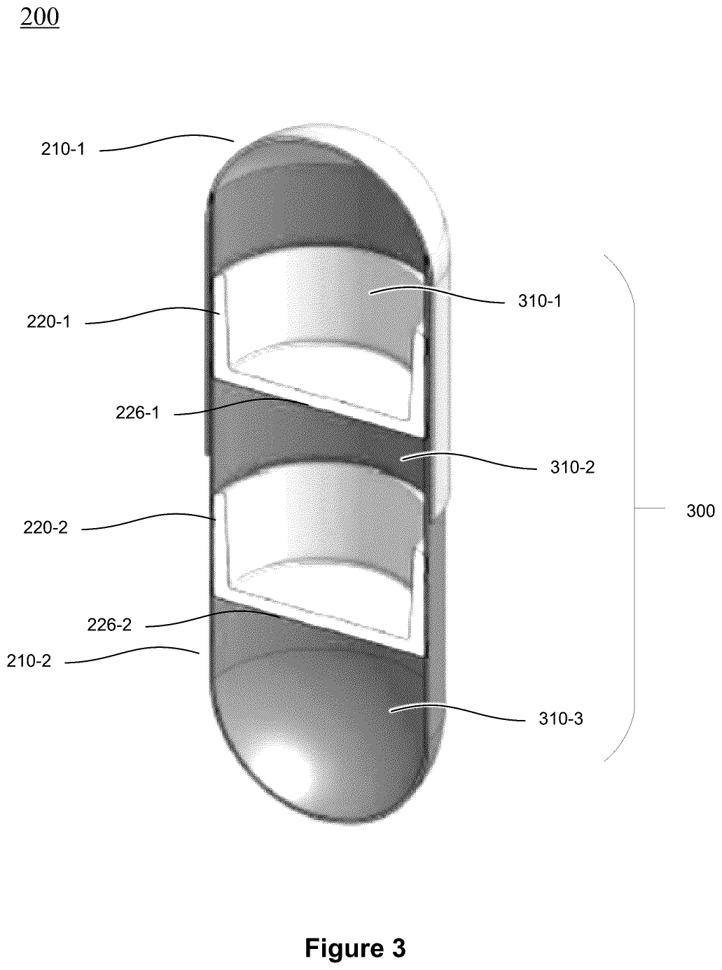

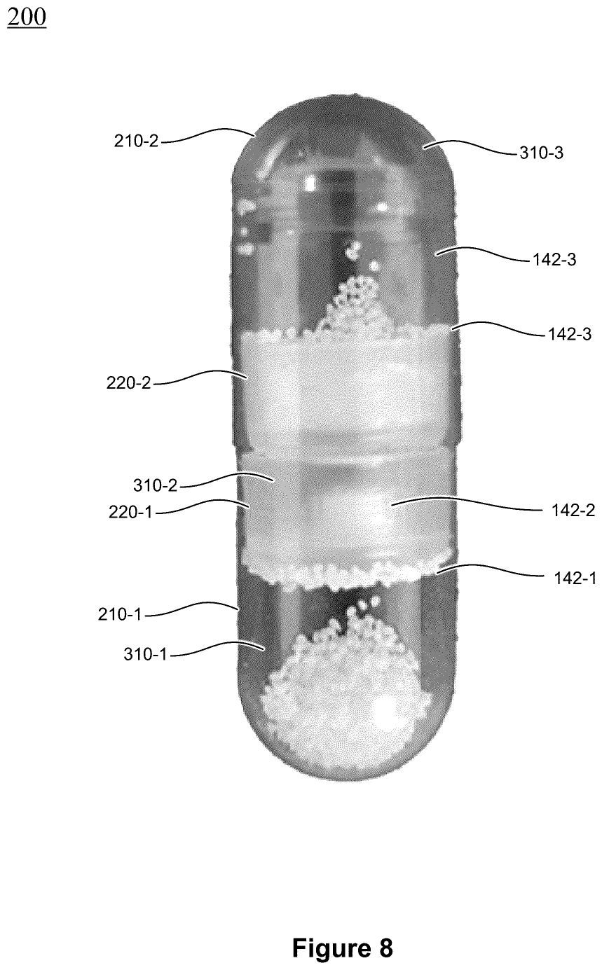

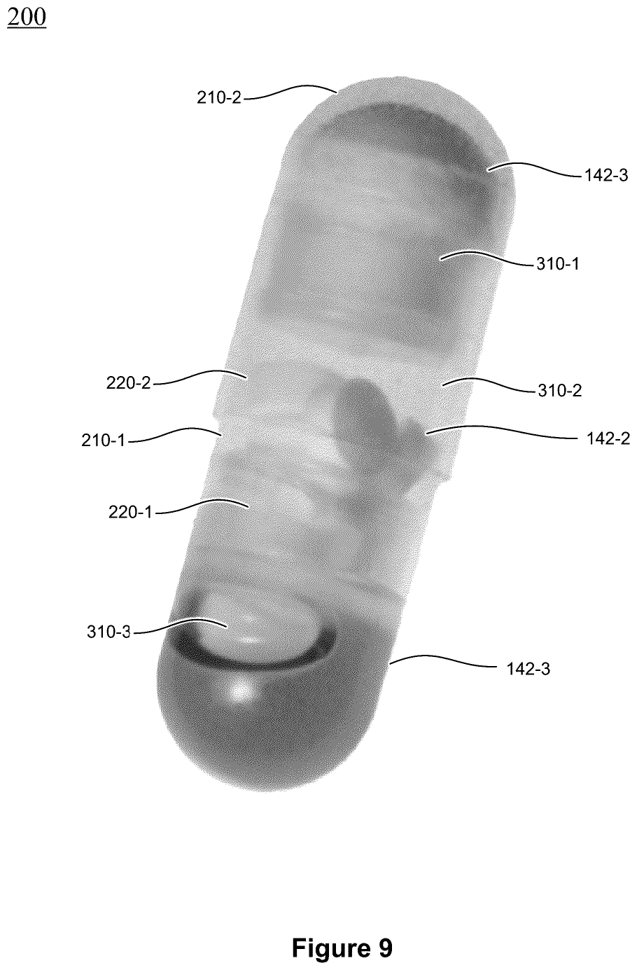

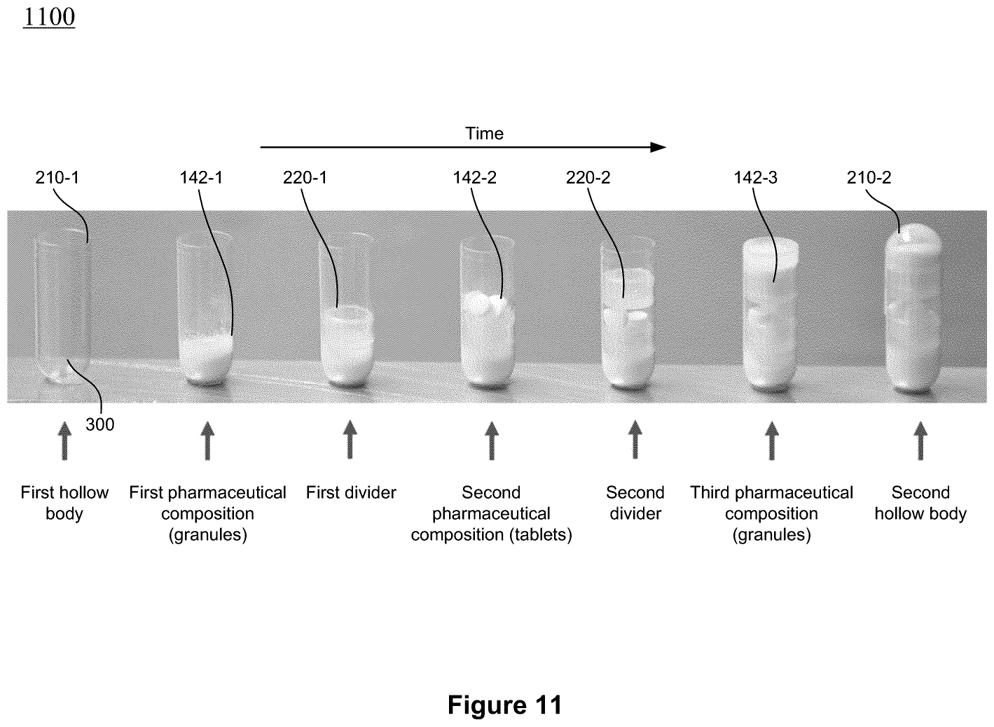

FIG. 1 depicts a block diagram of distributed computer system 100 according to some embodiments of the present disclosure. The computer system at least facilitates communicating one or more instructions for manufacture a capsule (e.g., capsule 200 of FIG. 2, capsule 200 of FIG. 3, capsule 200 of FIG. 8, capsule 200 of FIG. 9, etc.), such as respective hollow body of the capsule (e.g., first hollow body 210-1 of FIG. 2, second hollow body 210-2 of FIG. 8, etc.) and/or one or more dividers (e.g., first divider 200-1 of FIG. 2, second divider 200-2 of FIG. 11, etc.). For instance, in some embodiments, the computer system includes an additive manufacture system, which is utilized to manufacture the capsule. For instance, in some embodiments, the additive manufacturing system is selected from the group consisting of: binder jetting, material extrusion, material jetting, polyjet, powder bed (e.g., powder bed fusion), sheet lamination, direct energy deposition, and vat photopolymerization.

In some embodiments, communication network 106 optionally includes the Internet, one or more local area networks (LANs), one or more wide area networks (WANs), other types of networks, or a combination of such networks.

Examples of communication networks 106 include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

In various embodiments, computer system 100 includes one or more processing units (CPUs) 174, a network or other communications interface 112, and a memory 192.

In some embodiments, the computer system includes user interface 178. The user interface typically includes display 182 for presenting media, such as a graphical representation of a capsule or a view of a camera during a manufacture of the capsule. In some embodiments, the display is integrated within the computer systems (e.g., housed in the same chassis as the CPU and the memory). In some embodiments, the computer system includes one or more input device(s) 180, which allow a subject to interact with the computer system. In some embodiments, the one or more input devices include a keyboard, a mouse, and/or other input mechanisms. Alternatively, or in addition, in some embodiments, the display includes a touch-sensitive surface (e.g., where display is a touch-sensitive display or computer system includes a touch pad).

The memory includes high-speed random access memory, such as DRAM, SRAM, DDR RAM, or other random access solid state memory devices, and optionally also includes non-volatile memory, such as one or more magnetic disk storage devices, optical disk storage devices, flash memory devices, or other non-volatile solid state storage devices. The memory may optionally include one or more storage devices remotely located from the CPU(s). The memory, or alternatively the non-volatile memory device(s) within memory, includes a non-transitory computer readable storage medium. Access to the memory by other components of the computer system, such as the CPU(s), is, optionally, controlled by a controller (e.g., controller 188 of FIG. 1). In some embodiments, the memory can include mass storage that is remotely located with respect to the CPU(s). In other words, some data stored in the memory 192 may in fact be hosted on devices that are external to the computer system 100, but that can be electronically accessed by the computer system over an Internet, intranet, or other form of network 106 or electronic cable using network interface.

In some embodiments, the memory of the computer system stores:
operating system 108 (e.g., ANDROID, iOS, DARWIN, RTXC, LINUX, UNIX, OS X, WINDOWS, or an embedded operating system such as VxWorks) that includes procedures for handling various basic system services;
electronic address 110 associated with the computer system 100 that identifies the computer system 100 (e.g., within the communication network 106);
capsule generator module 120 for controlling manufacture of a capsule 200 using one or more instruments, such as an additive manufacture system, associated with the computer system 100;
subject profile data store 130 that stores a user profile for each of a plurality of subjects, each respective user profile including information about a corresponding subject, such as a mailing address and/or an order history for a provision of a capsule; and
pharmaceutical composition library 140 that stores a record of a plurality of pharmaceutical compositions 142 (e.g., pharmaceutical composition 142-1, pharmaceutical composition 142-2, . . . , pharmaceutical composition 142-A of FIG. 1) that can be utilized with a capsule 200.

As indicated above, electronic address 110 is associated with computer system 100. The electronic address is utilized to at least uniquely identify the computer system from other devices and components of the distributed system, such as other devices having access to the communications network. For instance, in some embodiments, the electronic address is utilized to receive a request from a remote device to manufacture.

In some embodiments, the computer system includes capsule generator module 120 for controlling manufacture of capsule 200. For instance, in some embodiments, the computer system includes an additive manufacture system, whose operation is controlled through the capsule generator module. As a non-limiting example, in some embodiments, the capsule generator module communications one or more instructions to the additive manufacture system to facilitate manufacture of respective hollow body 210 and/or respective divider 220 of the capsule. For instance, in response to the one or more instructions generated by the capsule generator module, a portion of the additive manufacture system traverses a region and conducts a fabrication of the capsule. As a non-limiting example, in some embodiments, the additive manufacture system utilizes a melt extrusion method that is used to form a three-dimensional structure in a layer-by-layer sequence (e.g., a sequence of traversing a plurality of two-dimensional layers. As another non-limiting example, in some embodiments, the additive manufacture system includes a vat photopolymerization mechanism. Accordingly, the capsule generator module communications one or more instructions to the vat photopolymerization mechanism associated with an intensity of light associated with the vat photopolymerization mechanism, an exposure time of light associated with the vat photopolymerization mechanism, a supply of a pre-polymer solution of the vat photopolymerization mechanism, a supply of a photo-initiator associated with the vat photopolymerization mechanism, and the like. However, the present disclosure is not limited thereto.

In some embodiments, the computer system includes subject profile data store 130, which stores a respective user profile for each subject in a plurality of subjects. The respective user profile includes information about a corresponding subject in the plurality of subjects, such as a mailing address associated with the corresponding subject, an electronic address associated with the corresponding subject, an adverse event history associated with the corresponding subject, a configuration of a capsule associated with the corresponding subject, one or more pharmaceuticals compositions associated with the corresponding subject, and the like.

In some embodiments, the computer system includes pharmaceutical composition library 140. The pharmaceutical composition library stores a record of pharmaceutical compositions 142. In some embodiments, the record of a respective pharmaceutical composition includes information about one or more indications of the respective pharmaceutical composition, one or more dosage forms of the pharmaceutical composition (e.g., first amlodipine pharmaceutical composition 142-1 is available for inclusion in a capsule as a 2.5 mg tablet, a 5 mg tablet, a 10 mg tablet, or a combination thereof, second pharmaceutical composition 142-2 is available for inclusion in the capsule as a 100 mg tablet or a 100 mL fluid, etc.), one or more adverse reactions associated with consumption of the respective pharmaceutical composition, and the like. Accordingly, each pharmaceutical composition is a consumable good.

Furthermore, in some embodiments, the record of the respective pharmaceutical composition includes information about one or more dimensional constraints of the respective pharmaceutical composition. Each dimensional constraint describes an ability to partition (e.g., sub-divide) the respective pharmaceutical composition into one or more dosages (e.g., a particular mass, a particular volume, etc.). In some embodiments, the one or more dimensional constraints for the respective pharmaceutical composition includes a number of dosage forms of the respective pharmaceutical composition within a dosage of the respective pharmaceutical composition (e.g., an available inventory of dosages). A dosage form of the respective pharmaceutical composition includes a unit of dosage the respective pharmaceutical composition. Accordingly, in some embodiments, the capsule generation module modifies an amount of the dosage of the respective pharmaceutical composition within a unique interior volume of the pharmaceutical composition by increasing, or similarly decreasing, the number of dosage forms of the respective pharmaceutical composition within the capsule to form a dosage of the pharmaceutical composition. For instance, consider a first pharmaceutical composition provided in a tablet dosage form that is commercially available as a 10 mg tablet dosage or a 25 mg tablet dosage. In some embodiments, a dosage of the first pharmaceutical composition is from about 10 mg to about 250 mg. Accordingly, the dimensional constraint of the first pharmaceutical composition is 10 mg or 20 mg and provides a dosage of 10 or more mg increasing from 20 mg upwards in multiples of 5 mg (e.g., 25 mg, 30 mg, 35 mg, etc.), such that the dimensional constraint arises from commercially realizable combinations of the 10 mg and the 25 mg dosage of the first pharmaceutical composition within the range of the dosage of the first pharmaceutical composition. As a non-limiting example, in some embodiments, a second pharmaceutical composition is metoprolol succinate. A dimensional constraint of the second pharmaceutical composition is that metoprolol succinate is commercially available in dosages of 25 mg, 50 mg, 100 mg, or 200 mg. In some embodiments, a dosage of the second pharmaceutical composition is from about 25 mg to about 200 mg. Accordingly, in some embodiments, the dosage of the second pharmaceutical composition within a capsule is 150 mg (e.g., a combination of the 25 mg and the 100 mg dosage within the capsule). Furthermore, in some embodiments, the dimensional constraint includes a minimum size of the respective pharmaceutical composition. In some embodiments, the minimum serving size of the respective pharmaceutical composition includes a functional limitation of an instrument utilized in quantifying the pharmaceutical composition for a respective capsule. For instance, if the instrument includes a scale to determine a mass with a precision of 0.1 g, the scale imparts a dimensional constraint of 0.1 grams (g) for the respective pharmaceutical composition that requires use of the scale for forming the respective capsule. In some embodiments, the dimensional constraint of the respective pharmaceutical composition includes a functional limitation of the respective pharmaceutical composition, such as a mass of a minimum effective dosage of the respective pharmaceutical composition and/or a maximum effective dosage of the respective pharmaceutical composition. However, one of skill in the art will appreciate that the present disclosure is not limited thereto.

In some embodiments, the pharmaceutical composition library stores: greater than 100 pharmaceutical compositions; greater than 500 pharmaceutical compositions; greater than 1,000 pharmaceutical compositions; greater than 10,000 pharmaceutical compositions; greater than 100,000 pharmaceutical compositions; greater than 1 million pharmaceutical compositions; or greater than 1 billion pharmaceutical compositions.

In some embodiments, the pharmaceutical composition library, optionally with the capsule generation module, is utilized to determine (e.g., devise, method 1100 of FIG. 11, etc.) a single unit dosage formulation of a pharmaceutical composition for a selected subject to prevent, treat, or ameliorate a selected condition of the subject (e.g., a hypertension condition, a coronary artery disease, a systolic heart failure condition, a heart failure with reduced ejection fraction (HFrEF) condition, a combination thereof, etc.). In some embodiments, the pharmaceutical composition library, optionally with the capsule generation module, is utilized to determine an appropriate dosages of two or more agents of use to prevent, treat, or ameliorate the condition taking into consideration the release rate of a first pharmaceutical composition in a first unique interior volume, and the size of this volume. In some embodiments, the pharmaceutical composition library, optionally with the capsule generation module, is utilized to take into consideration the release rate of a second pharmaceutical composition in a second unique interior volume, and the size of this volume. In some embodiments, the pharmaceutical composition library, optionally with the capsule generation module, is utilized to determine an effective dosage of each of the first and second pharmaceutical compositions. Furthermore, in some embodiments, the pharmaceutical composition library, optionally with the capsule generation module, is utilized to is utilized to determine appropriate dosages of two or more agents of use to prevent, treat or ameliorate the condition taking into consideration the release rate of a first pharmaceutical composition in a first unique interior volume, and the size of this volume.

In some embodiments, the pharmaceutical composition library, optionally with the capsule generation module, is utilized to utilized to take into consideration the release rate of a second pharmaceutical composition in a second unique interior volume, and the size of this volume. Moreover, in some embodiments, the pharmaceutical composition library, optionally with the capsule generation module, is utilized to determine an effective dosage of each of the first and second pharmaceutical composition.

In some embodiments, the pharmaceutical composition library, optionally with the capsule generation module, is utilized to fulfill an order for a capsule of the present disclosure, such as by communicating a provision of the capsule to an address associated with a subject of a remote device. However, the present disclosure is not limited thereto.

Each of the above identified modules and applications correspond to a set of executable instructions for performing one or more functions described above and the methods described in the present disclosure (e.g., the computer-implemented methods and other information processing methods described herein; etc.). These modules (e.g., sets of instructions) need not be implemented as separate software programs, procedures or modules, and thus various subsets of these modules are, optionally, combined or otherwise re-arranged in various embodiments of the present disclosure. In some embodiments, the memory optionally stores a subset of the modules and data structures identified above. Furthermore, in some embodiments, the memory 192 stores additional modules and data structures not described above.

It should be appreciated that the computer system of FIG. 1 is only one example of a computer system, and that the computer system optionally has more or fewer components than shown, optionally combines two or more components, or optionally has a different configuration or arrangement of the components. The various components shown in FIG. 1 are implemented in hardware, software, firmware, or a combination thereof, including one or more signal processing and/or application specific integrated circuits.

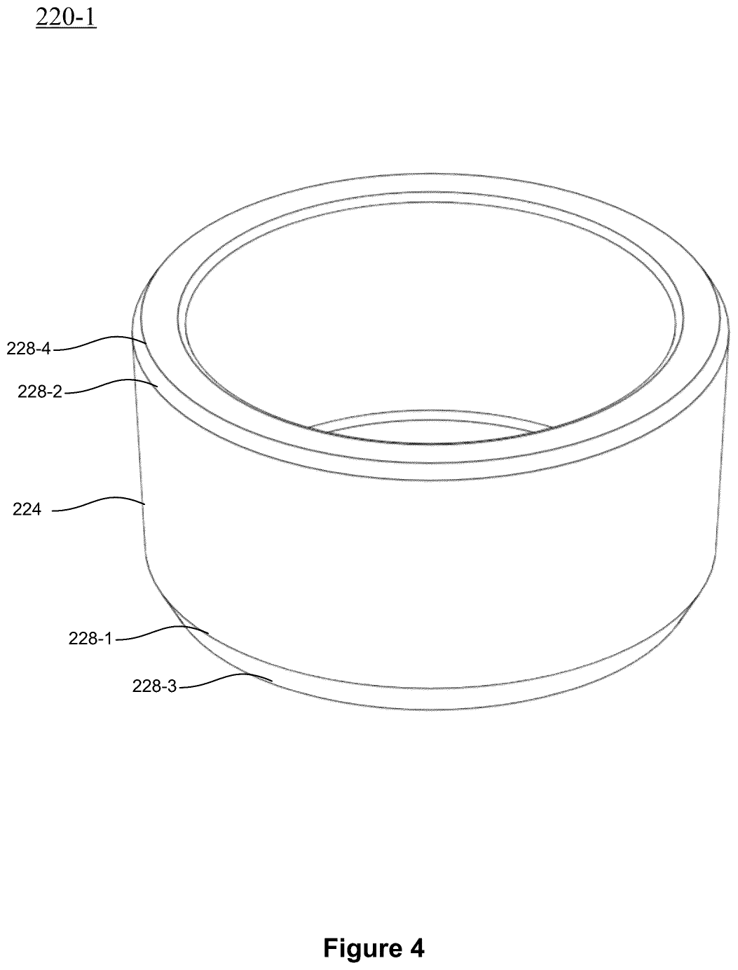

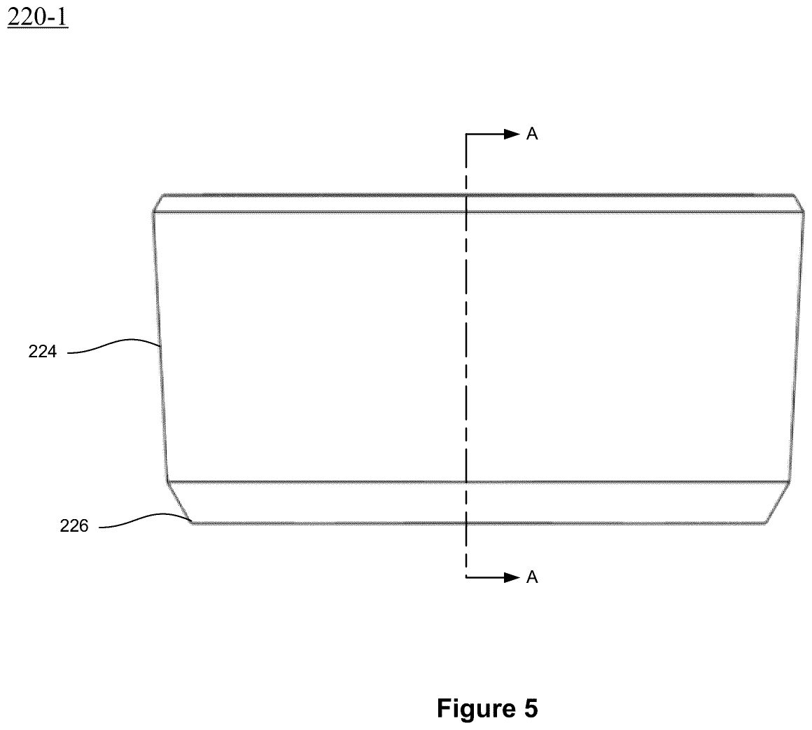

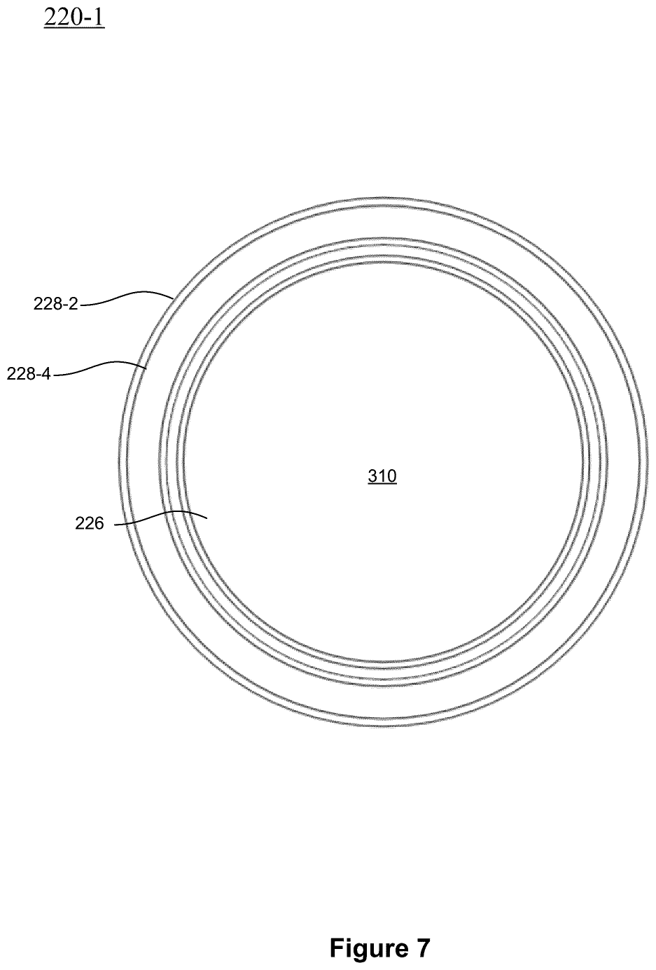

Now that a general topology of the distributed computer system 100 has been described in accordance with various embodiments of the present disclosures, details regarding a capsule in accordance with FIGS. 2 through 9 will be described.

In some embodiments, the present disclosure is directed to providing capsule 200.

In some embodiments, a capsule of the present disclosure has a structure as described in U.S. Pat. No. 10,456,975 B2, entitled “Mutli-Compartment Capsule,” issued Oct. 29, 2019; Melocchi et al., 2018, “Industrial Development of a 3D-Printed Nutraceutical Delivery Platform in the Form of a Multicompartment HPC Capsule,” AAPS PharmSciTech, 19(8), pg. 3343-3354, each of which is hereby incorporated by reference in its entirety for all purposes.

Referring to FIGS. 2 through 9, there is depicted an exemplary capsule in accordance with some embodiments of the present disclosure. In some embodiments, the capsule is a coated capsule, an uncoated capsule, an osmotic capsule, a multi-layered capsule, or the like. As a non-limiting example, in some embodiments, the osmotic capsule releases at least one pharmaceutical composition in the plurality of pharmaceutical compositions at a controlled rate by a process of osmotic bursting over a period of time.

As illustrated, capsule 200 includes at least two hollow bodies, such as first hollow body 210-1 and second hollow body 210-2. However, the present disclosure is not limited thereto. For instance, in some embodiments, the capsule includes three or more hollow bodies, five or more hollow bodies, ten or more hollow bodies, or the like.

In some embodiments, the first and second hollow bodies are substantially identical in shape, or symmetric, to each other. For instance, in some embodiments, each respective hollow body has a substantially cylindrical shape including an open end portion and a closed hemispherical end portion. However, the present disclosure is not limited thereto. In some embodiments, the first hollow body has the substantially cylindrical shape including the open end portion and the closed hemispherical end portion, and a third hollow body has the substantially cylindrical shape including opposing open end portions, such as a loop or band, which allows for extending a length of the capsule by disposing the third hollow body interposing between at least the first hollow body and the second hollow body.

Furthermore, in some embodiments, the capsule further includes a line of symmetry about an axis of the capsule (e.g., longitudinal axis, a central axis, a traversal axis, etc.). In some embodiments, the capsule has an hourglass shape, an ellipsoid shape, or a cylinder shape with hemispheres (e.g., a spherocylindrical shape). However, the present disclosure is not limited thereto. For instance, in some embodiments, the capsule has a non-uniform shape. In a further embodiment, the shape of the capsule is asymmetric about one or more axes, which enabling a subject to determine an orientation of the capsule by touch. Additionally, in some embodiments, a cross-section of the capsule is circular, oblong, square, rectangular, hexagonal, symmetrical about a first axis, or non-symmetrical about the first axis.

In some embodiments, the first hollow body and the second hollow body are configured to couple together. For instance, in some embodiments, the second hollow body is configured to couple to the first hollow body, which collectively form an interior volume of the capsule. Moreover, in some embodiments, the first hollow body is configured to couple to the second hollow body, which collectively form the interior volume of the capsule. For instance, in some embodiments, the first hollow body and/or the second hollow body includes one or more fastening mechanisms, such as an interlocking surface. As a non-limiting example, in some embodiments, the first hollow body include a male interlocking surface and the second hollow body includes a female interlocking surface configured to accommodate the male interlocking surface, which couples the second hollow body is configured to couple to the first hollow body. In some embodiments, the first hollow body and the second hollow body are configured to couple together telescopically. Furthermore, in some embodiments, each hollow body includes a closed end portion, which allows the capsule to have the interior volume sealed from an exterior environment when the first hollow body and the second hollow body are coupled together. Accordingly, in some such embodiments, the coupling of the second hollow body to the first hollow body seals the interior volume of the capsule form an environment.

In some embodiments, the closed end portion of the first end portion or the second end portion includes a radius of curvature greater than one. For instance, in some embodiments, the closed end portion includes a convex curvature. In some embodiments, the convex curvature extends from a first portion of the closed end portion to a second end portion of the closed end portion, which includes an apex of the closed end portion. In some embodiments, the capsule includes an edge interposing between the sidewall and the closed end portion.

Accordingly, in some embodiments, the interior volume of the capsule is in a range of from about 0.1 milliliters (mL) to about 1.5 mL. For instance, in some embodiments, the interior volume of the capsule 200 is about 0.13 mL, about 0.21 mL, about 0.30 mL, about 0.37 mL, about 0.50 mL, about 0.68 mL, about 0.95 mL, or about 1.37 mL. In some embodiments, the interior volume of the capsule 200 is at least 0.13 mL, at least 0.21 mL, at least 0.30 mL, at least 0.37 mL, at least 0.50 mL, at least 0.68 mL, at least 0.95 mL, or at least 1.37 mL. In some embodiments, the interior volume of the capsule 200 is at most 0.13 mL, at most 0.21 mL, at most 0.30 mL, at most 0.37 mL, at most 0.50 mL, at most 0.68 mL, at most 0.95 mL, or at most 1.37 mL. However, the present disclosure is not limited thereto.

In some embodiments, the capsule has an overall length (e.g., from a first end portion of the first hollow body to a second end portion of the second hollow body) in a range of from 50 mm to about 5 mm (e.g., 26.1 mm, 25.3 mm, 23.4 mm, 21.6 mm, 19.4 mm, 17.6 mm, 15.7 mm, 14.3 mm, 11.1 mm, etc.). In some embodiments, the overall length of the capsule is at least 50 mm, at least 26.1 mm, at least 25.3 mm, at least 23.4 mm, at least 21.6 mm, at least 19.4 mm, at least 17.6 mm, at least 15.7 mm, at least 14.3 mm, or at least 11.1 mm. In some embodiments, the overall length of the capsule is at most 50 mm, at most 26.1 mm, at most 25.3 mm, at most 23.4 mm, at most 21.6 mm, at most 19.4 mm, at most 17.6 mm, at most 15.7 mm, at most 14.3 mm, or at most 11.1 mm.

Furthermore, in some embodiments, the first hollow body all has a thickness (e.g., wall thickness) in range of from 0.01 mm to 1 mm (e.g., 0.6 mm). In some embodiments, the first hollow body has a thickness of at least 0.01 mm, at least 0.05 mm, at least 0.1 mm, at least 0.15 mm, at least 0.2 mm, at least 0.3 mm, at least 0.4 mm, at least 0.5 mm, at least 0.6 mm, at least 0.7 mm, at least 0.8 mm, at least 0.9 mm, or at least 1 mm. In some embodiments, the first hollow body has a thickness of at most 0.01 mm, at most 0.05 mm, at most 0.1 mm, at most 0.15 mm, at most 0.2 mm, at most 0.3 mm, at most 0.4 mm, at most 0.5 mm, at most 0.6 mm, at most 0.7 mm, at most at most 0.8 mm, at most 0.9 mm, or at most 1 mm.

In some embodiments, scaling the dimensions of the capsule (e.g., a respective hollow body of the capsule, a respective divider of the capsule, etc.) described herein results in a larger or smaller capsule with substantially the same ratios as described herein. In some embodiments, scaling the dimensions of the capsule (e.g., a respective hollow body of the capsule, a respective divider of the capsule, etc.) described herein results in a larger or smaller capsule with ratios different from those described herein.

Referring briefly to FIGS. 8 and 9, in some embodiments, capsule 200 includes a plurality of dividers, such as first divider 220-1 and second divider 220-2. However, the present disclosure is not limited thereto. For instance, in some embodiments, the capsule includes 1 divider, at least 2 dividers, at least 3 dividers, at least 4 dividers, at least 5 dividers, at least 6 dividers, at least 7 dividers, at least 8 dividers, at least 9 dividers, at least 10 dividers, or at least 11 dividers. In some embodiments, the capsule includes 1 divider, at most 2 dividers, at most 3 dividers, at most 4 dividers, at most 5 dividers, at most 6 dividers, at most 7 dividers, at most 8 dividers, at most 9 dividers, at most 10 dividers, or at most 11 dividers. As a non-limiting example, in an exemplary embodiment, the capsule includes 2 dividers, 3 dividers, 4 dividers, 5 dividers, 6 dividers, or 7 dividers. Each divider is configured to partition the interior volume of the capsule to form one or more unique interior volumes. For instance, referring briefly to FIG. 3, capsule 200 includes interior volume 300 that accommodates first divider 220-1 partitioning the interior volume into first unique interior volume 310-1 and second unique interior volume 310-1. Moreover, second divider 220-1 further partitioning the interior volume into third unique interior volume 310-1. As such, a collective volume of the first divider, the second divider, the first unique interior volume, the second unique interior volume, and the third unique interior volume substantially equal, or equal, the interior volume. Accordingly, each divider forms one or more unique interior volumes of the capsule based on a position of a respective divider, such as an orientation of the respective divider and/or a relative position in the interior volume, and a unique circumferential surface of the interior volume.

In some embodiments, the fist divider has a first shape, and the second divider has a second shape. In some such embodiments, the first shape is the same, or substantially the same, as the second shape. In other embodiments, the first shape is different than the second shape. Accordingly, the first divider partitions the interior volume into a first unique interior volume and a second unique interior volume, and the second divider further partitions the interior volume into a third unique interior volume. In some embodiments, each respective unique interior volume of the capsule has a uniform size (e.g., all of the unique interior volumes have an volume of about 0.25 mL). In some embodiments, each respective unique interior volume of the capsule has a heterogenous size (e.g., some of the unique interior volumes have the volume of about 0.25 mL). However, the present disclosure is not limited thereto. As another non-limiting example, in some embodiments, the first divider partitions the interior volume into the first unique interior volume and the second unique interior volume, the second divider further partitions the interior volume into the third unique interior volume, and a third divider partitions the interior volume into the fourth unique interior volume of the capsule. One of skill in the art of the present disclosure will appreciate that a number of dividers, and, therefore, a corresponding number of unique interior volumes of a respective capsule is not limited thereto. For instance, in some embodiments, the respective capsule includes 4 unique interior volumes, 5 unique interior volumes, 6 unique interior volumes, 7 unique interior volumes, 8 unique interior volumes, 9 unique interior volumes, 10 unique interior volumes, 11 unique interior volumes, 12 unique interior volumes, 13 unique interior volumes, 14 unique interior volumes, or 15 unique interior volumes. In some embodiments, the respective capsule includes at least 4 unique interior volumes, at least 5 unique interior volumes, at least 6 unique interior volumes, at least 7 unique interior volumes, at least 8 unique interior volumes, at least 9 unique interior volumes, at least 10 unique interior volumes, at least 11 unique interior volumes, at least 12 unique interior volumes, at least 13 unique interior volumes, at least 14 unique interior volumes, or at least 15 unique interior volumes. In some embodiments, the respective capsule includes at most 4 unique interior volumes, at most 5 unique interior volumes, at most 6 unique interior volumes, at most 7 unique interior volumes, at most 8 unique interior volumes, at most 9 unique interior volumes, at most 10 unique interior volumes, at most 11 unique interior volumes, at most 12 unique interior volumes, at most 13 unique interior volumes, at most 14 unique interior volumes, or at most 15 unique interior volumes. By utilizing the divider, the capsule of the present disclosure has a physical medium separating at least a first pharmaceutical composition and a second pharmaceutical composition within the interior volume of the capsule.

In some embodiments, first divider 220-1 includes base 226. Furthermore, in some embodiments, the first divider includes sidewall 224 protruding from first circumferential edge portion 228-1 of the base. The sidewall of the first divider includes an inner surface, an outer surface, and second circumferential edge portion 228-2. In some embodiments, the inner surface is parallel to the outer surface. In some embodiments, the inner surface is cylindrical or substantially cylindrical, and the outer surface is cylindrical or substantially cylindrical. In some embodiments, the inner surface is configured to contact a respective pharmaceutical composition of a unique interior volume of the capsule, and the outer surface is configured to contact a unique circumferential surface of the capsule. Accordingly, the second circumferential edge portion is configured to continuously engage a unique circumferential surface of the capsule. From this, the first divider is secured within the interior volume of the capsule based on a position of the first divider and the unique circumferential surface of the capsule. Moreover, by continuously engage the unique circumferential surface of the capsule, the first divider provides a seal, which prevents a first pharmaceutical composition of a first unique interior volume and a second pharmaceutical composition of a second unique interior volume from interacting physically or chemically.

In some embodiments, the base of the first divider includes a planar surface. In some embodiments, the planar surface includes a mating mechanism, in which is configured to couple to a corresponding mating mechanism disposed on an upper end portion of a corresponding divider in the plurality of dividers. For instance, in some embodiments, the mating mechanism includes a protrusion or the planar surface that is configured to nest with an opening of the corresponding mating mechanism formed at an upper end portion of the corresponding divider. However, the present disclosure is not limited thereto.

In some embodiments, a thickness of the base of the first divider is in a range of from about 0.1 mm to about 1.5 mm (e.g., 0.2 mm, 0.8 mm, etc.). In some embodiments, a wall thickness of the sidewall of the first divider is in a range of from about 0.1 mm to about 1.5 mm (e.g., 0.2 mm, 0.8 mm, etc.). In some embodiments, the wall thickness of the sidewall of the first divider is at least 0.1 mm, at least 0.15 mm, at least 0.2 mm, at least 0.3 mm, at least 0.4 mm, at least 0.5 mm, at least 0.6 mm, at least 0.7 mm, at least 0.8 mm, at least 0.9 mm, at least 1 mm, at least 1.1 mm, at least 1.2 mm, at least 1.3 mm, at least 1.4 mm, or at least 1.5 mm. In some embodiments, the first hollow body has a thickness of at most 0.01 mm, at most 0.05 mm, at most 0.1 mm, at most 0.15 mm, at most 0.2 mm, at most 0.3 mm, at most 0.4 mm, at most 0.5 mm, at most 0.6 mm, at most 0.7 mm, at most at most 0.8 mm, at most 0.9 mm, at most 1 mm, at most 1.1 mm, at most 1.2 mm, at most 1.3 mm, at most 1.4 mm, or at most 1.5 mm.

In some embodiments, the wall thickness of the sidewall various across a length of the sidewall. For instance, in some embodiments, the wall thickness of the sidewall is a first thickness at a first end portion of the sidewall proximate the base and a second thickness at a second end portion proximate the second circumferential edge of the first divider, and the first thickness of the first end portion is greater than the second thickness of the second end portion. By varying the wall thickness, in some embodiments, the first divider is configured to allow bending of a portion of the first divider, which reduces material fatigue for the first divider, the first hollow body, the second hollow body, or a combination thereof. Furthermore, by allowing the bending of the portion of the first divider, the first divider pushes against the inner surface of the capsule, which allows for sealing the partitioning of the first unique interior volume and the second unique interior volume.

In some embodiments, the first divider has a frustum shape. For instance, in some embodiments, the frustrum shape is a conical frustum or a pyramidal frustrum. In some embodiments, an exterior surface of the first divider has a radius of curvature greater than zero. For instance, in some embodiments, an edge portion of the frustrum shape is rounded. The frustrum shape of the first divider allows for insertion into the first hollow body without risking permanent deformation to the first hollow body, such as forming a crack in a surface of the first hollow body when disposing the first divider. Moreover, in some embodiments, the frustum shape of the first divider forms an opening (e.g., third unique interior volume of FIG. 8) for accommodating a respective pharmaceutical composition. For instance, in some embodiments, the upper end portion of the first divider is configured to couple to the base of the second divider, which forms a closed unique interior volume from the opening. However, the present disclosure is not limited thereto. For instance, in some embodiments, the shape of the first divider is cylindrical, cubic, conical, disc, hexagonal, paraboloidal, a prism, substantially toroidal, or other symmetric or non-symmetric shape. In some embodiments, the shape of the first divider is truncated, such as a truncated hollow cone.

In some embodiments, the second circumferential edge portion deforms radially. For instance, in some embodiments, a first diameter of the second circumferential edge portion of the first divider is greater than or equal to a second diameter of the first hollow body and/or the second hollow body of the capsule. Accordingly, the second circumferential edge portion radially deforms inwardly towards a center of the first divider (e.g., a longitudinal axis of the first divider) to at least the second diameter in order to ensure a secure engagement of the first divider within the interior volume of the capsule. Thus, the first divider can bend, creating a compliant point of contact with the capsule. Moreover, in some embodiments, the compliant point of contact with the capsule is the unique circumferential surface of the capsule, which provides for a seal between the at least two unique interior volumes formed by the divider.

In some embodiments, the inner surface and/or the outer surface of the sidewall is generally concave or generally convex. However, the present disclosure is not limited thereto.

In some embodiments, the base further includes an upper surface and a lower surface. Moreover, in some embodiments, the upper surface and/or the lower surface of the base is generally concave or generally convex. In some embodiments, the upper surface and the lower surface of the base are parallel or substantially parallel. For instance, in some embodiments, the upper surface of the base includes a planar surface, which allows for a corresponding surface to engage the planar surface when disposing the first divider within the first hollow body or the second hollow body of the capsule.

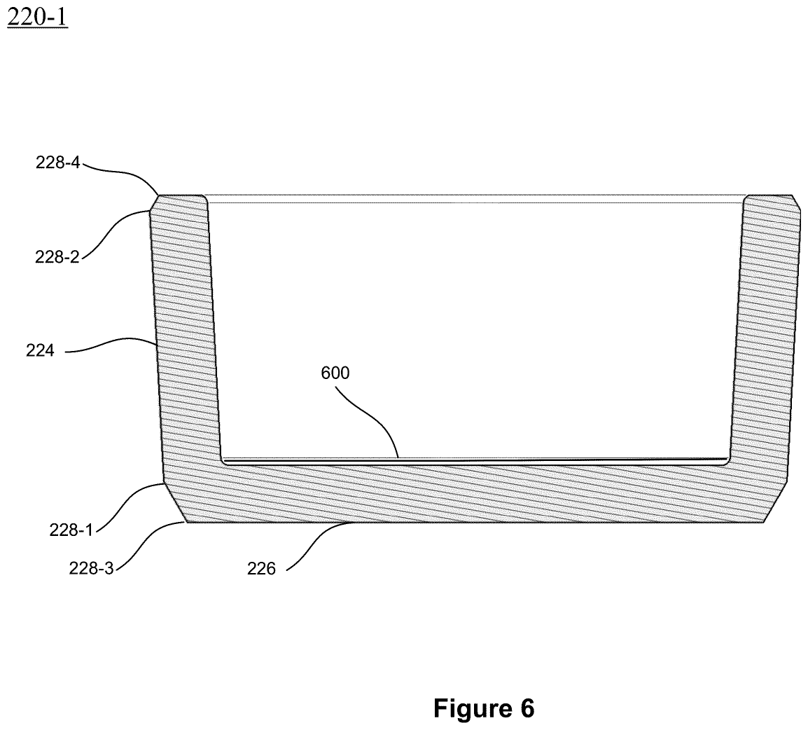

Referring briefly to FIG. 6, in some embodiments, capsule 200 further includes a layer of a material, such as layer of material 600. In some embodiments, the layer of material is disposed a surface of divider 220, such as on a portion of the upper surface of the divider. The layer of material is configured to prevent the upper surface of the divider from adhering to a different surface or face other than the layer of the material. In some embodiments, the layer of material is configured to prevent adherence of a substance from a respective pharmaceutical compound. In this way, the surface of the divider having the layer of material does not adhere to a portion of a respective pharmaceutical composition 142, a second divider, or hollow body 210 of the capsule. In some embodiments, the material includes a coefficient of friction in a range of from 0.1 to 0.8. In some embodiments, the coefficient of friction of the material is at least 0.1, at least 0.2, at least 0.3, at least 0.4, at least 0.5, at least 0.6, at least 0.7, or at least 0.8. In some embodiments, the coefficient of friction of the material is at most 0.1, at most 0.2, at most 0.3, at most 0.4, at most 0.5, at most 0.6, at most 0.7, or at most 0.8. For instance, in some embodiments, the layer of material includes a lubricant, or biocompatible material. Non-limiting examples of the layer of material include magnesium stearate and sodium stearyl fumarate. In some embodiments, the presence of the layer of material prevents a respective instrument (e.g., a pin) from getting stuck engaging the first divider when manufacturing the capsule (e.g., capsule 200 of FIG. 11), so the first divider stays in place when the respective instrument disengages the first divider.

In some embodiments, the second circumferential edge portion includes a chamfer or rounded edge. By using the chamfer or rounded edge, the second circumferential edge portion is easily inserted into the interior volume of the capsule. For instance, in some embodiments, if this the chamfer or rounded edge was absent from the second circumferential edge portion, a sharp edge of the divider might get caught in an edge portion of a respective hollow body, and crush the respective hollow body as the first divider is pushed down into the interior of the capsule (e.g., manufacture of capsule of FIG. 11). Accordingly, in some embodiments, the chamfer or rounded edge defines the maximum external diameter of the first divider at a point below an upper edge portion of the divider (e.g., the maximum external diameter is not located at an extremum in height of the first divider), which provides more protection, and therefore more stability, for the capsule.

Moreover, in some embodiments, fourth circumferential edge portion 228-4 includes a chamfer or rounded edge. In some embodiments, the chamfer or rounded edge of the fourth circumferential edge portion together with the chamfer or rounded edge of the second circumferential edge portion determines a maximum outer diameter or deformation of the first divider, which engages with the interior surface of the respective hollow body of the capsule. In some embodiments, a chamfer or rounded edge of the third circumferential edge portion together with the chamfer or rounded edge of the second circumferential edge portion determines a maximum outer diameter or deformation of the first divider, which engages with the interior surface of the respective hollow body of the capsule. In some embodiments, the chamfer or rounded edge of the first circumferential edge portion together with the chamfer or rounded edge of the second circumferential edge portion determines a maximum outer diameter or deformation of the first divider, which engages with the interior surface of the respective hollow body of the capsule. In some embodiments, the maximum outer diameter is substantially equal to a compressed diameter of the second circumferential edge portion (e.g., in comparison to a relaxed diameter of the second circumferential edge portion when the first divider is not subject to compressive forces from the first hollow body and/or the second hollow body).

In some embodiments, an outer diameter of the second circumferential edge portion exclusively engages the unique circumferential surface of the capsule. By having the second circumferential edge portion exclusively engages the unique circumferential surface of the capsule, the second circumferential edge portion is deformed inside the capsule. Accordingly, the second circumferential edge portion of the first divider is bent (e.g., elastically deformed in an inwardly radial direction) inside of the capsule. Moreover, due to the elastic deformation of the divider, the second circumferential edge portion has spring like behavior, which maintains contact (e.g., engagement) between the respective hollow body of the capsule and the first divider. Said otherwise, by utilizing an elastic material with a stiffness that allows for elastic deformation of the first divider, the first divider pushes against the inner surface of the capsule, which allows for sealing the partitioning of the first unique interior volume and the second unique interior volume. Moreover, by utilizing an elastic material with a stiffness that allows for elastic deformation of the first divider, material fatigue is reduced for the first divider, the first hollow body, the second hollow body, or a combination thereof. Accordingly, in some embodiments, the stiffness of the divider varies over different portions of the different. For instance, in some embodiments, the base of the first divider has a first stiffness and the sidewall of the first divider has a second stiffness, in which the first stiffness is greater than the second stiffness.

In some embodiments, the base of each divider further includes a third circumferential edge portion. In some embodiments, the second circumferential edge portion includes a first diameter, and the third circumferential edge portion includes a second diameter different from the first diameter. For instance, in some embodiments, the third circumferential edge portion is configured to couple to an upper end portion of a corresponding divider, which allows for the first divider to couple with the corresponding divider. However, the present disclosure is not limited thereto.

In some embodiments, the first circumferential edge portion, the second circumferential edge portion, the third circumferential edge portion, the fourth circumferential edge portion, or a combination thereof have a radius of curvature greater than zero. In some embodiments, the radius of curvature of the first circumferential edge portion, the second circumferential edge portion, and the third circumferential edge portion; the first circumferential edge portion, the second circumferential edge portion, and the fourth circumferential edge portion; or the fourth circumferential edge portion, the second circumferential edge portion, and the third circumferential edge portion do not include a radius of curvature that is the same as in another circumferential edge portion.

In some embodiments, the first hollow body or the second hollow body includes a third diameter greater than or equal to the second diameter. In some embodiments, the third diameter is less than or equal to the first diameter. In this way, the third diameter that is an inner dimension of the first hollow body or the second hollow body is less than or equal to the second diameter of the first divider, which is the largest diameter of the first divider, such that upon insertion of the first divider into the first hollow body or the second hollow body causes to engage the inner surface of the first hollow body or the second hollow body. However, the present disclosure is not limited thereto.

In some embodiments, a draft angle of the sidewall is greater than zero. In some embodiments, the draft angle is in a range of from about 0.1 degrees (°) to about 5°. In some embodiments, the draft angle of the sidewall is at least 0.1°, at least 0.5°, at least 1°, at least 1.5°, at least 2°, at least 2.5°, at least 3°, at least 3.5°, at least 4°, at least 4.5°, or at least 5°. In some embodiments, the draft angle of the sidewall is at most 0.1°, at most 0.5°, at most 1°, at most 1.5°, at most 2°, at most 2.5°, at most 3°, at most 3.5°, at most 4°, at most 4.5°, or at most 5°. In some embodiments, a first draft angle of the outer surface of the side wall allows the first divider to easily be inserted into a respective hollow body due to a slope imparted by the first draft angle on this outer surface. Accordingly, in some embodiments, only a circumferential edge portion upper part of the divider is actually in contact with the inner surface of the capsule. From this, in some such embodiments, the draft angle of the side wall of the capsule causes an outer dimension of the first divider to engage a surface of the respective hollow body (e.g., a substantially cylindrical surface) during insertion of the first divider.

In some embodiments, a characteristic length of the sidewall is in a range of from about 0.5 millimeters (mm) to about 30 mm. For instance, in some embodiments, the characteristic length of the side wall is about 2 mm, about 3 mm, about 5 mm, about 10 mm, about 15 mm, about 20 mm, about 25 mm (e.g., 24.9 mm), or about 30 mm. In some embodiments, the characteristic length of the side wall is at least 2 mm, at least 3 mm, at least 5 mm, at least 10 mm, at least 15 mm, at least 20 mm, at least 25 mm, or at least 30 mm. In some embodiments, the characteristic length of the side wall is at most 2 mm, at most 3 mm, at most 5 mm, at most 10 mm, at most 15 mm, at most 20 mm, at most 25 mm, or at most 30 mm. In some embodiments, a feature depth of the draft angle is in a range of from 0.2 mm to about 2 inches (e.g., 0.25 mm, 0.5 inches, etc.).

In some embodiments, the first hollow body, the second hollow body, the first divider, the second divider, or a combination thereof is transparent, translucent, or opaque. For instance, in some embodiments, the first hollow body, the second hollow body, the first divider, the second divider, or the combination thereof includes one or more cellulosic compounds, one or more acrylates, one or more starch ethers, one or more polyolefins, one or more pullulans, one or more carrageenan gelatins, or a combination thereof. By having the first hollow body, the second hollow body, the first divider, the second divider, or the combination thereof be transparent, translucent, or opaque, a subject can visually inspect the capsule and the respective pharmaceutical compositions accommodated therein to ensure quality control.

Furthermore, in some embodiments, the first divider and/or the second divider has a particular density that is less dense than a respective pharmaceutical composition in the plurality of pharmaceutical compositions having a liquid state (e.g., water), which ensures the first divider and/or the second divider does not sink below an upper surface of the respective pharmaceutical composition. Furthermore, in some embodiments, the first divider and/or the second divider is denser than of the respective pharmaceutical composition having the liquid state. In some in some embodiments, the first divider and/or the second divider is less dense than the respective pharmaceutical composition having the non-aqueous liquid state (e.g., oil). In some embodiments, the respective pharmaceutical composition is denser than the respective pharmaceutical composition having the non-aqueous liquid state.

In some embodiments, the first divider is a monolithic body. For instance, in some embodiments, the monolithic body of the first divider is a solid object that is formed as a single piece. As a non-limiting example, in some embodiments, the first divider is formed without any internal voids, cavities, or separate parts. However, the present disclosure is not limited thereto. Accordingly, in some embodiments, the first divider has a continuous structure (e.g., a seamless structure). By having the monolithic body, the first divider provides an improved seal against a surface of the capsule, while reducing risk of defect caused by material fatigue. Furthermore, via the monolithic body, in some embodiments, the first divider does not have an increased risk of mechanical failure since there is no assembly required to form the first divider or couple the first divider to the capsule aside from disposing the first divider within the interior volume of the capsule.

In some embodiments, the plurality of pharmaceutical compositions includes one or more solutions, one or more suspensions, one or more emulsions, one or more tablets (e.g., second pharmaceutical composition 142-2 of FIG. 8, second pharmaceutical composition 142-2 of FIG. 11, etc.), one or more capsules, one or more powders, one or more bead, one or more pellet, one or more granules (e.g., third pharmaceutical composition 142-3 of FIG. 8, first pharmaceutical composition 142-1 of FIG. 11, etc.), one or more solid dispersions, or combinations thereof. In some embodiments, the first divider is configured to maintain a solid state when interfacing with a respective pharmaceutical composition in the plurality of pharmaceutical compositions.

In some embodiments, the first divider is defined by a first orientation. Furthermore, in some embodiments, the capsule further includes a second divider defined by a second orientation. In some embodiments, the first orientation is different from the second orientation. In some embodiments, the first orientation is opposite, or opposes, the second orientation. However, the present disclosure is not limited thereto. For instance, in some embodiments, the first orientation and the second orientation are the same. Referring briefly to FIG. 8, in some embodiments, first divider 220-1 is deposed in a first orientation and second divider 220-2 is disposed in the first orientation such that an opening formed by the side wall faces a base of the second divider. Accordingly, the dividers form at least two larger unique interior volumes of the capsule (e.g., where an articulated handling robot can deposit granule pharmaceutical compositions 142-1 and 142-3) at extremes of the capsule (e.g., upper and lower end portions). Furthermore, in some embodiments, the orientation of the first and second dividers forms a smaller, third unique interior volume, such as if the first and second dividers have opposing orientations. In some such embodiments, the third unique interior volume includes a cavity below the base of the second divider. In FIG. 8, the third unique interior volume includes a single tablet of a pharmaceutical composition (e.g., a white shadow inside the first divider). Accordingly, the third unique interior volume provides sufficient interior volume within the capsule for a respective pharmaceutical composition, and frees up more interior volume within the capsule for the other unique interior volumes partitioned by a respective divider in the capsule. However, the present disclosure is not limited thereto.

In some embodiments, the first divider, the first hollow body, the second hollow body, or a combination thereof includes a material selected from the group consisting of: ethyl cellulose, hydroxypropyl cellulose, hydroxypropyl methyl cellulose, hydroxypropyl methyl cellulose acetate succinate, various types of methacrylic acid copolymers, polyethylene oxide, polyvinyl alcohol, polyvinyl alcohol-polyethylene glycol graft copolymer, and polyvinyl caprolactam-polyvinyl acetate-polyethylene glycol graft co-polymer. In some embodiments, the first divider, the first hollow body, the second hollow body, or a combination thereof includes an absorbable material, a biodegradable material, or a combination thereof. In some embodiments, the first divider, the first hollow body, the second hollow body, or a combination thereof includes a water soluble polymer, an erodible polymer, a degradable polymer, a swellable polymer, or a combination thereof. In some embodiments, the water soluble polymer is sparingly soluble, slightly soluble, very slightly soluble, or practically insoluble. However, the present disclosure is not limited thereto. For instance, in some embodiments, the divider 220 and/or the hollow body 210 is sparingly soluble, slightly soluble, very slightly soluble, or practically insoluble in a chloroform solution, an acetone solution, an alcohol, or the like. In some embodiments, a degree of polymerization of the first divider, the first hollow body, the second hollow body, or a combination is in a range of from 40 to 2,000. For instance, in some embodiments, the degree of polymerization of the first divider, the first hollow body, the second hollow body, or the combination is about 48, about 56, about 59, about 77, about 79, about 100, about 115 (e.g., 116), about 150, about 225, about 335, about 460, about 690, about 720, about 860, about 1,260, about 1,600, or about 2,000 (e.g., 2010).

In some embodiments, the capsule encapsulates a single pharmaceutical composition. As will be discussed further herein, in an exemplary embodiment, the capsule encapsulates more than one different pharmaceutical composition. For example, the capsule can encapsulate 1, 2, 3, 4, or more different pharmaceutical compositions. In various embodiments, each unique pharmaceutical composition occupies a unique volume within the capsule.

In various embodiments, the capsule includes two or more volumes within the capsule, each containing the same pharmaceutical composition. In an exemplary embodiment, the pharmaceutical composition is present in a first volume and a second volume in a first amount and a second amount and the first and second amounts are different amounts. In some embodiments, the first and second volumes are defined by capsule materials having different rates of dissolution or erosion, thereby providing a capsule with two separate delivery profiles for the same pharmaceutical composition. In capsules providing more than one dissolution, erosion, i.e., pharmaceutical composition release profile, the pharmaceutical compositions can be present in the same or different amounts.

As will be appreciated, this same strategy can be applied to capsules including two or more different pharmaceutical compositions or a combination of volumes loaded with the same or different pharmaceutical compositions. Selecting capsule components for a target dissolution or erosion rate and, hence, release rate of a selected pharmaceutical composition, is within the ability of those of ordinary skill in the art without resort to undue experimentation.

In an exemplary embodiment of a capsule designed according to the strategy above, the invention provides a capsule releasing from a first volume the pharmaceutical composition rapidly (“burst”) and from a second volume at a slower rate (“controlled release”).

An exemplary capsule includes a plurality of pharmaceutical compositions. In some embodiments, respective pharmaceutical composition 142 in the plurality of pharmaceutical compositions occupies a unique interior volume of capsule 200. In some embodiments, first pharmaceutical composition 142-1 in the plurality of pharmaceutical compositions occupies the first unique interior volume, second pharmaceutical composition 142-2 in the plurality of pharmaceutical compositions occupies the second unique interior volume, and third pharmaceutical composition 142-3 in the plurality of pharmaceutical compositions occupies the third unique interior volume.

For instance, in some embodiments, the respective pharmaceutical composition is selected from the drug class group: a calcium channel blocker (CCB) pharmaceutical composition; an angiotensin converting enzyme inhibitor (ACEi) pharmaceutical composition; an angiotensin II receptor blocker (ARB) pharmaceutical composition; a thiazide pharmaceutical composition; a beta-adrenergic receptor antagonist (BB) pharmaceutical composition; a potassium-sparing diuretic (K-sparing diuretic) pharmaceutical composition; a potassium-wasting diuretic (K-wasting diuretic); a lipid-lowering agent pharmaceutical composition; an anti-platelet pharmaceutical composition; an anti-anginal pharmaceutical composition; anti-arrhythmic pharmaceutical composition; and a dietary supplement. An exemplary capsule encapsulates a pharmaceutical composition from two or more of these classes.

Further, in some such embodiments, the CCB pharmaceutical composition is selected from of: amlodipine; diltiazem; and a combination thereof.

In some embodiments, the ACEi pharmaceutical composition is selected from the group: lisinopril; ramipril; enalapril; and a combination thereof.

In some embodiments, the ARB pharmaceutical composition is selected from the group consisting of: losartan; valsartan; candesartan; olmesartan; and a combination thereof.

In some embodiments, the thiazide pharmaceutical composition is selected from the group consisting of: chlorthalidone; hydrochlorothiazide, and a combination thereof.

In some embodiments, the BB pharmaceutical composition is selected from the group consisting of metoprolol succinate; carvedilol; bisoprolol; propranolol (e.g., propranolol LA1); and a combination thereof.

In some embodiments, the K-sparing diuretic pharmaceutical composition is spironolactone.

In some embodiments, the K-wasting diuretic pharmaceutical composition is selected from: furosemide; torsemide; bumetanide; metolazone; and a combination thereof.

In some embodiments, the lipid-lowering pharmaceutical composition is selected from the group consisting of: atorvastatin; rosuvastatin; pravastatin; simvastatin; ezetimibe; and a combination thereof.

In some embodiments, the anti-platelet pharmaceutical composition is selected from the group consisting of: aspirin; prasugrel; and a combination thereof.

In some embodiments, the anti-anginal pharmaceutical composition is isosorbide, such as isosorbide mononitrate.

In some embodiments, the anti-arrhythmic pharmaceutical composition is amiodarone.

In some embodiments, the dietary supplement is a particular nutrient, a particular nutritional product, a chemical, or any other composition for which nutritional limit data is available. Accordingly, “dietary supplement” refers to individual dietary ingredients within nutritional products as well as particular nutritional products themselves, and a combination thereof. Non-limiting examples of dietary supplements include, but are not limited to, one or more minerals, one or more amino acids, one or more herbs, one or more botanicals, as well as other substances that can be used to supplement the diet and that are found in nutritional products. For instance, in some embodiments, the dietary supplement includes or is selected from calcium, iodine, iron, magnesium, potassium, ubiquinone (e.g., coenzyme Q10), zinc, and a combination thereof.

In some embodiments, the first pharmaceutical composition is amlodipine. In some such embodiments, a dosage of amlodipine is of from about 1 milligram (mg) to about 10 mg. In some such embodiments, the dosage of amlodipine is about 2.5 mg, about 5 mg, or about 10 mg.

In some embodiments, the second pharmaceutical composition is losartan. In some such embodiments, a dosage of losartan is from about 25 mg to about 100 mg. In some embodiments, the dosage of valsartan is about 25 mg, about 50 mg, or about 100 mg. In some embodiments, the third pharmaceutical composition is hydrochlorothiazide. In some such embodiments, a dosage of hydrochlorothiazide is from about 12.5 mg to about 50 mg. In some such embodiments, the dosage of hydrochlorothiazide is about 12.5 mg, about 25 mg, or about 50 mg.

In some embodiments, the first pharmaceutical composition of the capsule is a dosage of about 10 mg of amlodipine, the second pharmaceutical composition of the capsule is a dosage of about 100 mg of losartan, and the third pharmaceutical composition of the capsule is a dosage of about 25 mg of hydrochlorothiazide.

In some embodiments, the first pharmaceutical composition is amlodipine. In some such embodiments, the dosage of amlodipine is of from about 1 mg to about 10 mg. In some such embodiments, the dosage of amlodipine is about 2.5 mg, about 5 mg, or about 10 mg. In some embodiments, the second pharmaceutical composition is valsartan. In some such embodiments, the dosage of valsartan is of from about 20 mg to about 320 mg. In some embodiments, the dosage of valsartan is about 40 mg, about 80 mg, about 160 mg, or about 320 mg. In some embodiments, the third pharmaceutical composition is hydrochlorothiazide. In some such embodiments, the dosage of hydrochlorothiazide is from about 12.5 mg to about 50 mg. In some such embodiments, the dosage of hydrochlorothiazide is about 12.5 mg, about 25 mg, or about 50 mg.

In some embodiments, the first pharmaceutical composition of the capsule is a dosage of about 10 mg of amlodipine, the second pharmaceutical composition of the capsule is a dosage of about 320 mg of valsartan, and the third pharmaceutical composition of the capsule is a dosage of about 25 mg of hydrochlorothiazide.

In some embodiments, the first pharmaceutical composition is amlodipine. In some such embodiments, the dosage of amlodipine is of from about 1 mg to about 10 mg. In some such embodiments, the dosage of amlodipine is about 2.5 mg, about 5 mg, or about 10 mg. In some embodiments, the second pharmaceutical composition is lisinopril. In some such embodiments, the dosage of lisinopril is of from about 2.5 mg to about 40 mg. In some embodiments, the dosage of lisinopril is about 2.5 mg, about 5 mg, about 10 mg, about 20 mg, about 30 mg, or about 40 mg. In some embodiments, the third pharmaceutical composition is hydrochlorothiazide. In some such embodiments, the dosage of hydrochlorothiazide is from about 12.5 mg to about 50 mg. In some such embodiments, the dosage of hydrochlorothiazide is about 12.5 mg, about 25 mg, or about 50 mg.

In some embodiments, the first pharmaceutical composition of the capsule is the dosage of about 10 mg of amlodipine, the second pharmaceutical composition of the capsule is the dosage of about 5 mg of lisinopril, and the third pharmaceutical composition of the capsule is the dosage of about 25 mg of hydrochlorothiazide.

In some embodiments, the first pharmaceutical composition is amlodipine. In some such embodiments, the dosage of amlodipine is of from about 1 mg to about 10 mg. In some such embodiments, the dosage of amlodipine is about 2.5 mg, about 5 mg, or about 10 mg. In some embodiments, the second pharmaceutical composition is losartan. In some such embodiments, the dosage of losartan is of from about 25 mg to about 100 mg. In some embodiments, the dosage of losartan is about 25 mg, about 50 mg, or about 100 mg. In some embodiments, the third pharmaceutical composition is chlorthalidone. In some such embodiments, the dosage of chlorthalidone is from about 15 mg to about 25 mg. In some such embodiments, the dosage of chlorthalidone is about 15 mg, or about 25 mg.

In some embodiments, the first pharmaceutical composition of the capsule is the dosage of about 10 mg of amlodipine, the second pharmaceutical composition of the capsule is the dosage of about 50 mg of losartan, and the third pharmaceutical composition of the capsule is the dosage of about 12.5 mg of chlorthalidone.

In some embodiments, the first pharmaceutical composition is metoprolol succinate. In some such embodiments, the dosage of metoprolol succinate is of from about 25 mg to about 200 mg. In some such embodiments, the dosage of metoprolol succinate is about 25 mg, about 50 mg, about 100 mg, or about 200 mg. In some embodiments, the second pharmaceutical composition is lisinopril. In some such embodiments, the dosage of lisinopril is of from about 2.5 mg to about 40 mg. In some embodiments, the dosage of lisinopril is about 2.5 mg, about 5 mg, about 10 mg, about 20 mg, about 30 mg, or about 40 mg. In some embodiments, the third pharmaceutical composition is hydrochlorothiazide. In some such embodiments, the dosage of hydrochlorothiazide is from about 12.5 mg to about 50 mg. In some such embodiments, the dosage of hydrochlorothiazide is about 12.5 mg, about 25 mg, or about 50 mg.

In some embodiments, the first pharmaceutical composition of the capsule is the dosage of about 10 mg of metoprolol succinate, the second pharmaceutical composition of the capsule is the dosage of about 40 mg of lisinopril, and the third pharmaceutical composition of the capsule is the dosage of about 50 mg of hydrochlorothiazide.

In some embodiments, the first pharmaceutical composition is amlodipine. In some such embodiments, the dosage of amlodipine is of from about 1 mg to about 10 mg. In some such embodiments, the dosage of amlodipine is about 2.5 mg, about 5 mg, or about 10 mg. In some embodiments, the second pharmaceutical composition is lisinopril. In some such embodiments, the dosage of lisinopril is of from about 2.5 mg to about 40 mg. In some embodiments, the dosage of lisinopril is about 2.5 mg, about 5 mg, about 10 mg, about 20 mg, about 30 mg, or about 40 mg. In some embodiments, the third pharmaceutical composition is hydrochlorothiazide. In some such embodiments, the dosage of hydrochlorothiazide is from about 12.5 mg to about 50 mg. In some such embodiments, the dosage of hydrochlorothiazide is about 12.5 mg, about 25 mg, or about 50 mg. In some embodiments, a fourth pharmaceutical composition is spironolactone. In some such embodiments, a dosage of spironolactone is of from about 25 mg to about 100 mg. In some embodiments, the dosage of spironolactone is about 25 mg, about 50 mg, or about 100 mg. In some embodiments, the first pharmaceutical composition of the capsule is the dosage of about 10 mg of amlodipine, the second pharmaceutical composition of the capsule is the dosage of about 50 mg of lisinopril, the third pharmaceutical composition of the capsule is the dosage of about 25 mg of hydrochlorothiazide, and the fourth pharmaceutical composition of the capsule is the dosage of about 12.5 mg of spironolactone.

In some embodiments, the first pharmaceutical composition is ramipril. In some such embodiments, the dosage of ramipril is of from about 1.25 mg to about 20 mg (e.g., about 17.5 mg of ramipril). In some such embodiments, the dosage of ramipril is about 1.25 mg, about 2.5 mg, about 5 mg, or about 10 mg. In some embodiments, the second pharmaceutical composition is bisoprolol. In some such embodiments, the dosage of bisoprolol is of from about 2.5 mg to about 20 mg. In some embodiments, the dosage of bisoprolol is about 2.5 mg, about 5 mg, about 10 mg, or about 20 mg. In some embodiments, the third pharmaceutical composition is hydrochlorothiazide. In some such embodiments, the dosage of hydrochlorothiazide is from about 12.5 mg to about 50 mg. In some such embodiments, the dosage of hydrochlorothiazide is about 12.5 mg, about 25 mg, or about 50 mg. In some embodiments, the first pharmaceutical composition of the capsule is the dosage of about 10 mg of ramipril, the second pharmaceutical composition of the capsule is the dosage of about 5 mg of bisoprolol, and the third pharmaceutical composition of the capsule is the dosage of about 25 mg of hydrochlorothiazide.

In some embodiments, the first pharmaceutical composition is metoprolol succinate. In some such embodiments, the dosage of metoprolol succinate is of from about 25 mg to about 200 mg. In some such embodiments, the dosage of metoprolol succinate is about 25 mg, about 50 mg, about 100 mg, or about 200 mg. In some embodiments, the second pharmaceutical composition is lisinopril. In some such embodiments, the dosage of lisinopril is of from about 2.5 mg to about 40 mg. In some embodiments, the dosage of lisinopril is about 2.5 mg, about 5 mg, about 10 mg, about 20 mg, about 30 mg, or about 40 mg. In some embodiments, the third pharmaceutical composition is aspirin. In some such embodiments, the dosage of aspirin is from about 50 mg to about 1,000 mg (e.g., about 325 mg of aspirin). In some such embodiments, the dosage of aspirin is about 81 mg, about 150 mg, about 300 mg, about 600 mg, or about 800 mg. In some embodiments, the fourth pharmaceutical composition is atorvastatin. In some such embodiments, the dosage of atorvastatin is of from about 20 mg to about 100 mg. In some embodiments, the dosage of atorvastatin is about 20 mg, about 40 mg, about 60 mg, about 80 mg, about 100 mg. In some embodiments, the first pharmaceutical composition of the capsule is the dosage of about 150 mg of metoprolol, the second pharmaceutical composition of the capsule is the dosage of about 40 mg of lisinopril, the third pharmaceutical composition of the capsule is the dosage of about 81 mg of aspirin, and the fourth pharmaceutical composition of the capsule is the dosage of about 80 mg of atorvastatin.

In some embodiments, the first pharmaceutical composition is diltiazem. In some such embodiments, the dosage of diltiazem is of from about 120 mg to about 480 mg. In some such embodiments, the dosage of diltiazem is about 120 mg, about 180 mg, about 240 mg, about 360 mg, or about 480 mg. In some embodiments, the second pharmaceutical composition is valsartan. In some such embodiments, the dosage of valsartan is of from about 20 mg to about 320 mg. In some embodiments, the dosage of valsartan is about 40 mg, about 80 mg, or about 160 mg. In some embodiments, the third pharmaceutical composition is aspirin. In some such embodiments, the dosage of aspirin is from about 50 mg to about 1,000 mg. In some such embodiments, the dosage of aspirin is about 81 mg, about 150 mg, about 300 mg, about 600 mg, or about 800 mg. In some embodiments, the fourth pharmaceutical composition is pravastatin. In some such embodiments, the dosage of pravastatin is of from about 20 mg to about 80 mg. In some embodiments, the dosage of pravastatin is about 20 mg, about 40 mg, about 60 mg, or about 80 mg. In some embodiments, a fifth pharmaceutical composition is ezetimibe. In some such embodiments, a dosage of ezetimibe is about 10 mg. In some embodiments, the first pharmaceutical composition of the capsule is the dosage of about 180 mg of diltiazem, the second pharmaceutical composition of the capsule is the dosage of about 40 mg of valsartan, the third pharmaceutical composition of the capsule is the dosage of about 81 mg of aspirin, the fourth pharmaceutical composition of the capsule is the dosage of about 20 mg of pravastatin, and the fifth pharmaceutical composition of the capsule is the dosage of about 10 mg of ezetimibe.

In some embodiments, the first pharmaceutical composition is diltiazem. In some such embodiments, the dosage of diltiazem is of from about 120 mg to about 480 mg. In some such embodiments, the dosage of diltiazem is about 120 mg, about 180 mg, about 240 mg, about 360 mg, or about 480 mg. In some embodiments, the second pharmaceutical composition is metoprolol succinate. In some such embodiments, the dosage of metoprolol succinate is of from about 25 mg to about 200 mg. In some such embodiments, the dosage of metoprolol succinate is about 25 mg, about 50 mg, about 100 mg, or about 200 mg. In some embodiments, the third pharmaceutical composition is aspirin. In some such embodiments, the dosage of aspirin is from about 50 mg to about 1,000 mg. In some such embodiments, the dosage of aspirin is about 81 mg, about 150 mg, about 300 mg, about 600 mg, or about 800 mg. In some embodiments, the fourth pharmaceutical composition is lisinopril. In some such embodiments, the dosage of lisinopril is of from about 2.5 mg to about 40 mg. In some embodiments, the dosage of lisinopril is about 2.5 mg, about 5 mg, about 10 mg, about 20 mg, about 30 mg, or about 40 mg. In some embodiments, the fifth pharmaceutical composition is atorvastatin. In some such embodiments, the dosage of atorvastatin is of from about 10 mg to about 80 mg. In some embodiments, the dosage of atorvastatin is about 10 mg, about 20 mg, about 40 mg, or about 80 mg. In some embodiments, a sixth pharmaceutical composition is isosorbide mononitrate. In some such embodiments, a dosage of isosorbide mononitrate is of from about 30 mg to about 120 mg. In some embodiments, the dosage of isosorbide mononitrate is about 30 mg, about 60 mg, or about 120 mg. In some embodiments, the first pharmaceutical composition of the capsule is the dosage of about 180 mg of diltiazem, the second pharmaceutical composition of the capsule is the dosage of about 100 mg of metoprolol succinate, the third pharmaceutical composition of the capsule is the dosage of about 81 mg of aspirin, the fourth pharmaceutical composition of the capsule is the dosage of about 20 mg of lisinopril, the fifth pharmaceutical composition of the capsule is the dosage of about 80 mg of atorvastatin, and the sixth pharmaceutical composition of the capsule is the dosage of about 90 mg of isosorbide mononitrate.

In some embodiments, the first pharmaceutical composition is diltiazem. In some such embodiments, the dosage of diltiazem is of from about 120 mg to about 480 mg. In some such embodiments, the dosage of diltiazem is about 120 mg, about 180 mg, about 240 mg, about 360 mg, or about 480 mg. In some embodiments, the second pharmaceutical composition is metoprolol succinate. In some such embodiments, the dosage of metoprolol succinate is of from about 25 mg to about 200 mg. In some such embodiments, the dosage of metoprolol succinate is about 25 mg, about 50 mg, about 100 mg, or about 200 mg. In some embodiments, the third pharmaceutical composition is aspirin. In some such embodiments, the dosage of aspirin is from about 50 mg to about 1,000 mg. In some such embodiments, the dosage of aspirin is about 81 mg, about 150 mg, about 300 mg, about 600 mg, or about 800 mg. In some embodiments, the fourth pharmaceutical composition is enalapril. In some such embodiments, the dosage of enalapril is of from about 2.5 mg to about 20 mg (e.g., about 37.5 mg of enalapril). In some embodiments, the dosage of enalapril is about 2.5 mg, about 5 mg, about 10 mg, or about 20 mg.

In some embodiments, the fifth pharmaceutical composition is rosuvastatin. In some such embodiments, the dosage of rosuvastatin is of from about 5 mg to about 40 mg. In some embodiments, the dosage of rosuvastatin is about 5 mg, about 10 mg, about 20 mg, or about 40 mg. In some embodiments, the sixth pharmaceutical composition is ezetimibe. In some such embodiments, the dosage of ezetimibe is about 10 mg. In some embodiments, a seventh pharmaceutical composition is isosorbide mononitrate. In some such embodiments, a dosage of isosorbide mononitrate is of from about 30 mg to about 120 mg. In some embodiments, the dosage of isosorbide mononitrate is about 30 mg, about 60 mg, or about 120 mg. In some embodiments, the first pharmaceutical composition of the capsule is the dosage of about 120 mg of diltiazem, the second pharmaceutical composition of the capsule is the dosage of about 200 mg of metoprolol succinate, the third pharmaceutical composition of the capsule is the dosage of about 81 mg of aspirin, the fourth pharmaceutical composition of the capsule is the dosage of about 5 mg of enalapril, the fifth pharmaceutical composition of the capsule is the dosage of about 20 mg of rosuvastatin, the sixth pharmaceutical composition of the capsule is the dosage of about 10 mg of ezetimibe, and the seventh pharmaceutical composition of the capsule is the dosage of about 30 mg of isosorbide mononitrate.

In some embodiments, the first pharmaceutical composition is diltiazem. In some such embodiments, the dosage of diltiazem is of from about 120 mg to about 480 mg. In some such embodiments, the dosage of diltiazem is about 120 mg, about 180 mg, about 240 mg, about 360 mg, or about 480 mg. In some embodiments, the second pharmaceutical composition is carvedilol phosphate. In some such embodiments, the dosage of carvedilol phosphate is of from about 10 mg to about 80 mg. In some such embodiments, the dosage of carvedilol phosphate is about 10 mg, about 20 mg, about 40 mg, about 80 mg. In some embodiments, the third pharmaceutical composition is aspirin. In some such embodiments, the dosage of aspirin is from about 50 mg to about 1,000 mg. In some such embodiments, the dosage of aspirin is about 81 mg, about 150 mg, about 300 mg, about 600 mg, or about 800 mg. In some embodiments, the fourth pharmaceutical composition is losartan. In some such embodiments, the dosage of losartan is of from about 25 mg to about 100 mg. In some embodiments, the dosage of losartan is about 25 mg, about 50 mg, or about 100 mg. In some embodiments, the fifth pharmaceutical composition is rosuvastatin. In some such embodiments, the dosage of rosuvastatin is of from about 5 mg to about 40 mg. In some embodiments, the dosage of rosuvastatin is about 5 mg, about 10 mg, about 20 mg, or about 40 mg. In some embodiments, the sixth pharmaceutical composition is ezetimibe. In some such embodiments, the dosage of ezetimibe is about 10 mg. In some embodiments, the first pharmaceutical composition of the capsule is the dosage of about 120 mg of diltiazem, the second pharmaceutical composition of the capsule is the dosage of about 25 mg of carvedilol phosphate, the third pharmaceutical composition of the capsule is the dosage of about 81 mg of aspirin, the fourth pharmaceutical composition of the capsule is the dosage of about 25 mg of losartan, the fifth pharmaceutical composition of the capsule is the dosage of about 40 mg of rosuvastatin, and the sixth pharmaceutical composition of the capsule is the dosage of about 10 mg of ezetimibe.

In some embodiments, the first pharmaceutical composition is metoprolol succinate. In some such embodiments, the dosage of metoprolol succinate is of from about 25 mg to about 200 mg. In some such embodiments, the dosage of metoprolol succinate is about 25 mg, about 50 mg, about 100 mg, or about 200 mg. In some embodiments, the second pharmaceutical composition is lisinopril. In some such embodiments, the dosage of lisinopril is of from about 2.5 mg to about 40 mg. In some embodiments, the dosage of lisinopril is about 2.5 mg, about 5 mg, about 10 mg, about 20 mg, about 30 mg, or about 40 mg. In some embodiments, the third pharmaceutical composition is prasugrel. In some such embodiments, the dosage of prasugrel is from about 5 mg to about 10 mg. In some such embodiments, the dosage of prasugrel is about 5 mg, or about 10 mg. In some embodiments, the fourth pharmaceutical composition is atorvastatin. In some such embodiments, the dosage of atorvastatin is of from about 20 mg to about 100 mg. In some embodiments, the dosage of atorvastatin is about 20 mg, about 40 mg, about 60 mg, about 80 mg, or about 100 mg. In some embodiments, the first pharmaceutical composition of the capsule is the dosage of about 150 mg of metoprolol, the second pharmaceutical composition of the capsule is the dosage of about 40 mg of lisinopril, the third pharmaceutical composition of the capsule is the dosage of about 5 mg or about 10 mg of prasugrel, and the fourth pharmaceutical composition of the capsule is the dosage of about 80 mg of atorvastatin.

In some embodiments, the first pharmaceutical composition is diltiazem. In some such embodiments, the dosage of diltiazem is of from about 120 mg to about 480 mg. In some such embodiments, the dosage of diltiazem is about 120 mg, about 180 mg, about 240 mg, about 360 mg, or about 480 mg. In some embodiments, the second pharmaceutical composition is valsartan. In some such embodiments, the dosage of valsartan is of from about 20 mg to about 320 mg. In some embodiments, the dosage of valsartan is about 40 mg, about 80 mg, or about 160 mg. In some embodiments, the third pharmaceutical composition is prasugrel. In some such embodiments, the dosage of prasugrel is from about 5 mg to about 10 mg. In some such embodiments, the dosage of prasugrel is about 5 mg or about 10 mg. In some embodiments, the fourth pharmaceutical composition is pravastatin. In some such embodiments, the dosage of pravastatin is of from about 20 mg to about 80 mg. In some embodiments, the dosage of pravastatin is about 20 mg, about 40 mg, about 60 mg, or about 80 mg. In some embodiments, a fifth pharmaceutical composition is ezetimibe. In some such embodiments, a dosage of ezetimibe is about 10 mg. In some embodiments, the first pharmaceutical composition of the capsule is the dosage of about 180 mg of diltiazem, the second pharmaceutical composition of the capsule is the dosage of about 40 mg of valsartan, the third pharmaceutical composition of the capsule is the dosage of about 5 mg or about 10 mg of prasugrel, the fourth pharmaceutical composition of the capsule is the dosage of about 20 mg of pravastatin, and the fifth pharmaceutical composition of the capsule is the dosage of about 10 mg of ezetimibe.

In some embodiments, the first pharmaceutical composition is diltiazem. In some such embodiments, the dosage of diltiazem is of from about 120 mg to about 480 mg. In some such embodiments, the dosage of diltiazem is about 120 mg, about 180 mg, about 240 mg, or about 360 mg, about 480 mg. In some embodiments, the second pharmaceutical composition is metoprolol succinate. In some such embodiments, the dosage of metoprolol succinate is of from about 25 mg to about 200 mg. In some such embodiments, the dosage of metoprolol succinate is about 25 mg, about 50 mg, about 100 mg, or about 200 mg. In some embodiments, the third pharmaceutical composition is prasugrel. In some such embodiments, the dosage of prasugrel is from about 5 mg to about 10 mg. In some such embodiments, the dosage of prasugrel is about 5 mg, or about 10 mg. In some embodiments, the fourth pharmaceutical composition is lisinopril. In some such embodiments, the dosage of lisinopril is of from about 2.5 mg to about 40 mg. In some embodiments, the dosage of lisinopril is about 2.5 mg, about 5 mg, about 10 mg, about 20 mg, about 30 mg, or about 40 mg. In some embodiments, the fifth pharmaceutical composition is atorvastatin. In some such embodiments, the dosage of atorvastatin is of from about 10 mg to about 80 mg. In some embodiments, the dosage of atorvastatin is about 10 mg, about 20 mg, about 40 mg, or about 80 mg. In some embodiments, a sixth pharmaceutical composition is isosorbide mononitrate. In some such embodiments, a dosage of isosorbide mononitrate is of from about 30 mg to about 120 mg. In some embodiments, the dosage of isosorbide mononitrate is about 30 mg, about 60 mg, or about 120 mg. In some embodiments, the first pharmaceutical composition of the capsule is the dosage of about 180 mg of diltiazem, the second pharmaceutical composition of the capsule is the dosage of about 100 mg of metoprolol succinate, the third pharmaceutical composition of the capsule is the dosage of about 5 mg or about 10 mg of prasugrel, the fourth pharmaceutical composition of the capsule is the dosage of about 20 mg of lisinopril, the fifth pharmaceutical composition of the capsule is the dosage of about 80 mg of atorvastatin, and the sixth pharmaceutical composition of the capsule is the dosage of about 90 mg of isosorbide mononitrate.

In some embodiments, the first pharmaceutical composition is diltiazem. In some such embodiments, the dosage of diltiazem is of from about 120 mg to about 480 mg. In some such embodiments, the dosage of diltiazem is about 120 mg, about 180 mg, about 240 mg, about 360 mg, or about 480 mg. In some embodiments, the second pharmaceutical composition is metoprolol succinate. In some such embodiments, the dosage of metoprolol succinate is of from about 25 mg to about 200 mg. In some such embodiments, the dosage of metoprolol succinate is about 25 mg, about 50 mg, about 100 mg, or about 200 mg. In some embodiments, the third pharmaceutical composition is prasugrel. In some such embodiments, the dosage of prasugrel is from about 5 mg to about 10 mg. In some such embodiments, the dosage of prasugrel is about 5 mg or about 10 mg. In some embodiments, the fourth pharmaceutical composition is enalapril. In some such embodiments, the dosage of enalapril is of from about 2.5 mg to about 20 mg. In some embodiments, the dosage of enalapril is about 2.5 mg, about 5 mg, about 10 mg, or about 20 mg. In some embodiments, the fifth pharmaceutical composition is rosuvastatin. In some such embodiments, the dosage of rosuvastatin is of from about 5 mg to about 40 mg. In some embodiments, the dosage of rosuvastatin is about 5 mg, about 10 mg, about 20 mg, or about 40 mg. In some embodiments, the sixth pharmaceutical composition is ezetimibe. In some such embodiments, the dosage of ezetimibe is about 10 mg. In some embodiments, a seventh pharmaceutical composition is isosorbide mononitrate. In some such embodiments, a dosage of isosorbide mononitrate is of from about 30 mg to about 120 mg. In some embodiments, the dosage of isosorbide mononitrate is about 30 mg, about 60 mg, or about 120 mg. In some embodiments, the first pharmaceutical composition of the capsule is the dosage of about 120 mg of diltiazem, the second pharmaceutical composition of the capsule is the dosage of about 200 mg of metoprolol succinate, the third pharmaceutical composition of the capsule is the dosage of about 5 mg or about 10 mg of prasugrel, the fourth pharmaceutical composition of the capsule is the dosage of about 5 mg of enalapril, the fifth pharmaceutical composition of the capsule is the dosage of about 20 mg of rosuvastatin, the sixth pharmaceutical composition of the capsule is the dosage of about 10 mg of ezetimibe, and the seventh pharmaceutical composition of the capsule is the dosage of about 30 mg of isosorbide mononitrate.

In some embodiments, the first pharmaceutical composition is diltiazem. In some such embodiments, the dosage of diltiazem is of from about 120 mg to about 480 mg. In some such embodiments, the dosage of diltiazem is about 120 mg, about 180 mg, about 240 mg, about 360 mg, or about 480 mg. In some embodiments, the second pharmaceutical composition is carvedilol phosphate. In some such embodiments, the dosage of carvedilol phosphate is of from about 10 mg to about 80 mg. In some such embodiments, the dosage of carvedilol phosphate is about 10 mg, about 20 mg, about 40 mg, or about 80 mg. In some embodiments, the third pharmaceutical composition is prasugrel. In some such embodiments, the dosage of prasugrel is from about 5 mg to about 10 mg. In some such embodiments, the dosage of prasugrel is about 5 mg or about 10 mg. In some embodiments, the fourth pharmaceutical composition is losartan. In some such embodiments, the dosage of losartan is of from about 25 mg to about 100 mg. In some embodiments, the dosage of losartan is about 25 mg, about 50 mg, or about 100 mg. In some embodiments, the fifth pharmaceutical composition is rosuvastatin. In some such embodiments, the dosage of rosuvastatin is of from about 5 mg to about 40 mg. In some embodiments, the dosage of rosuvastatin is about 5 mg, about 10 mg, about 20 mg, or about 40 mg. In some embodiments, the sixth pharmaceutical composition is ezetimibe. In some such embodiments, the dosage of ezetimibe is about 10 mg. In some embodiments, the first pharmaceutical composition of the capsule is the dosage of about 120 mg of diltiazem, the second pharmaceutical composition of the capsule is the dosage of about 25 mg of carvedilol phosphate, the third pharmaceutical composition of the capsule is the dosage of about 5 mg or about 10 mg of prasugrel, the fourth pharmaceutical composition of the capsule is the dosage of about 25 mg of losartan, the fifth pharmaceutical composition of the capsule is the dosage of about 40 mg of rosuvastatin, and the sixth pharmaceutical composition of the capsule is the dosage of about 10 mg of ezetimibe.

In some embodiments, the first pharmaceutical composition is metoprolol succinate. In some such embodiments, the dosage of metoprolol succinate is of from about 25 mg to about 200 mg. In some such embodiments, the dosage of metoprolol succinate is about 25 mg, about 50 mg, about 100 mg, or about 200 mg. In some embodiments, the second pharmaceutical composition is lisinopril. In some such embodiments, the dosage of lisinopril is of from about 2.5 mg to about 40 mg. In some embodiments, the dosage of lisinopril is about 2.5 mg, about 5 mg, about 10 mg, about 20 mg, about 30 mg, or about 40 mg. In some embodiments, the third pharmaceutical composition is spironolactone. In some such embodiments, the dosage of spironolactone is from about 12.5 mg to about 100 mg. In some such embodiments, the dosage of spironolactone is about 25 mg, about 50 mg, or about 100 mg. In some embodiments, the fourth pharmaceutical composition is furosemide. In some such embodiments, the dosage of furosemide is of from about 20 mg to about 200 mg (e.g., about 180 mg of furosemide). In some embodiments, the dosage of furosemide is about 20 mg, about 40 mg, or about 80 mg. In some embodiments, the first pharmaceutical composition of the capsule is the dosage of about 200 mg of metoprolol, the second pharmaceutical composition of the capsule is the dosage of about 40 mg of lisinopril, the third pharmaceutical composition of the capsule is the dosage of about 12.5 mg of spironolactone, and the fourth pharmaceutical composition of the capsule is the dosage of about 80 mg of furosemide.

In some embodiments, the first pharmaceutical composition is metoprolol succinate. In some such embodiments, the dosage of metoprolol succinate is of from about 25 mg to about 200 mg. In some such embodiments, the dosage of metoprolol succinate is about 25 mg, about 50 mg, about 100 mg, or about 200 mg. In some embodiments, the second pharmaceutical composition is eplerenone. In some such embodiments, the dosage of eplerenone is of from about 25 mg to about 50 mg. In some embodiments, the dosage of eplerenone is about 25 mg or about 50 mg. In some embodiments, the third pharmaceutical composition is torsemide. In some such embodiments, the dosage of torsemide is from about 5 mg to about 100 mg. In some such embodiments, the dosage of torsemide is about 5 mg, about 10 mg, about 20 mg, or about 100 mg. In some embodiments, the fourth pharmaceutical composition is amiodarone. In some such embodiments, the dosage of amiodarone is of from about 200 mg to about 1,200 mg. In some embodiments, the dosage of amiodarone is about 200 mg, about 400 mg, about 800 mg, or about 1,200 mg. In some embodiments, the first pharmaceutical composition of the capsule is the dosage of about 200 mg of metoprolol, the second pharmaceutical composition of the capsule is the dosage of about 25 mg of eplerenone, the third pharmaceutical composition of the capsule is the dosage of about 40 mg of torsemide, and the fourth pharmaceutical composition of the capsule is the dosage of about 400 mg of amiodarone.

In some embodiments, the first pharmaceutical composition is lisinopril. In some such embodiments, the dosage of lisinopril is of from about 2.5 mg to about 40 mg. In some embodiments, the dosage of lisinopril is about 2.5 mg, about 5 mg, about 10 mg, about 20 mg, about 30 mg, or about 40 mg. In some embodiments, the third pharmaceutical composition is spironolactone. In some embodiments, the second pharmaceutical composition is bisoprolol. In some such embodiments, the dosage of bisoprolol is from about 5 mg to about 10 mg. In some embodiments, the dosage of bisoprolol is about 5 mg or about 10 mg. In some such embodiments, the dosage of bumetanide is from about 0.5 mg to about 4 mg. In some such embodiments, the dosage of bumetanide is about 0.5 mg, about 1 mg, or about 2 mg. In some embodiments, the fourth pharmaceutical composition is metolazone. In some such embodiments, the dosage of metolazone is of from about 2.5 mg to about 10 mg. In some embodiments, the dosage of metolazone is about 2.5 mg, about 5 mg, or about 10 mg. In some embodiments, the first pharmaceutical composition of the capsule is the dosage of about 40 mg of lisinopril, the second pharmaceutical composition of the capsule is the dosage of about 10 mg of bisoprolol, the third pharmaceutical composition of the capsule is the dosage of about 2 mg of bumetanide, and the fourth pharmaceutical composition of the capsule is the dosage of about 5 mg of metolazone.

In some embodiments, the first pharmaceutical composition is metoprolol succinate. In some such embodiments, the dosage of metoprolol succinate is of from about 25 mg to about 200 mg. In some such embodiments, the dosage of metoprolol succinate is about 25 mg, about 50 mg, about 100 mg, or about 200 mg. In some embodiments, the second pharmaceutical composition is ramipril. In some such embodiments, the dosage of ramipril is of from about 1.25 mg to about 20 mg. In some embodiments, the dosage of ramipril is about 1.25 mg, about 2.5 mg, about 5 mg, or about 10 mg. In some such embodiments, the dosage of bumetanide is from about 0.5 mg to about 4 mg. In some such embodiments, the dosage of bumetanide is about 0.5 mg, about 1 mg, or about 2 mg. In some embodiments, the fourth pharmaceutical composition is amiodarone. In some such embodiments, the dosage of amiodarone is of from about 400 mg to about 1,200 mg. In some embodiments, the dosage of amiodarone is about 200 mg, about 400 mg, about 800 mg, or about 1,200 mg. In some embodiments, the first pharmaceutical composition of the capsule is the dosage of about 100 mg of metoprolol, the second pharmaceutical composition of the capsule is the dosage of about 5 mg of ramipril, the third pharmaceutical composition of the capsule is the dosage of about 4 mg of bumetanide, and the fourth pharmaceutical composition of the capsule is the dosage of about 400 mg of amiodarone.

In some embodiments, the first pharmaceutical composition is valsartan. In some such embodiments, the dosage of valsartan is of from about 20 mg to about 320 mg. In some embodiments, the dosage of valsartan is about 40 mg, about 80 mg, or about 160 mg. In some embodiments, the second pharmaceutical composition is carvedilol phosphate. In some such embodiments, the dosage of carvedilol phosphate is of from about 10 mg to about 80 mg. In some such embodiments, the dosage of carvedilol phosphate is about 10 mg, about 20 mg, or about 40 mg, about 80 mg. In some embodiments, the third pharmaceutical composition is spironolactone. In some such embodiments, the dosage of spironolactone is from about 12.5 mg to about 100 mg. In some such embodiments, the dosage of spironolactone is about 25 mg, about 50 mg, or about 100 mg. In some embodiments, the fourth pharmaceutical composition is torsemide. In some such embodiments, the dosage of torsemide is from about 5 mg to about 100 mg. In some such embodiments, the dosage of torsemide is about 5 mg, about 10 mg, about 20 mg, or about 100 mg. In some embodiments, the fifth pharmaceutical composition is metolazone. In some such embodiments, the dosage of metolazone is of from about 2.5 mg to about 10 mg. In some embodiments, the dosage of metolazone is about 2.5 mg, about 5 mg, or about 10 mg. In some embodiments, the sixth pharmaceutical composition is amiodarone. In some such embodiments, the dosage of amiodarone is of from about 400 mg to about 1,200 mg. In some embodiments, the dosage of amiodarone is about 200 mg, about 400 mg, about 800 mg, or about 1,200 mg. In some embodiments, the first pharmaceutical composition of the capsule is the dosage of about 80 mg of valsartan, the second pharmaceutical composition of the capsule is the dosage of about 25 mg of carvedilol phosphate, the third pharmaceutical composition of the capsule is the dosage of about 12.5 mg of spironolactone, the fourth pharmaceutical composition of the capsule is the dosage of about 80 mg of torsemide, the fifth pharmaceutical composition of the capsule is the dosage of about 2.5 mg of metolazone, and the sixth pharmaceutical composition of the capsule is the dosage of about 400 mg of amiodarone.

In some embodiments, the first pharmaceutical composition is aspirin. In some such embodiments, the dosage of aspirin is from about 50 mg to about 1,000 mg. In some such embodiments, the dosage of aspirin is about 81 mg, about 150 mg, about 300 mg, about 600 mg, or about 800 mg. In some embodiments, the second pharmaceutical composition is atorvastatin. In some such embodiments, the dosage of atorvastatin is of from about 10 mg to about 80 mg. In some embodiments, the dosage of atorvastatin is about 10 mg, about 20 mg, about 40 mg, or about 80 mg. In some embodiments, the third pharmaceutical composition is ezetimibe. In some such embodiments, the dosage of ezetimibe is about 10 mg. In some embodiments, the fourth pharmaceutical composition is metoprolol succinate. In some such embodiments, the dosage of metoprolol succinate is of from about 25 mg to about 200 mg. In some such embodiments, the dosage of metoprolol succinate is about 25 mg, about 50 mg, about 100 mg, or about 200 mg. In some embodiments, the fifth pharmaceutical composition is lisinopril. In some such embodiments, the dosage of lisinopril is of from about 2.5 mg to about 40 mg. In some embodiments, the dosage of lisinopril is about 2.5 mg, about 5 mg, about 10 mg, about 20 mg, about 30 mg, or about 40 mg. In some embodiments, the sixth pharmaceutical composition is hydrochlorothiazide. In some such embodiments, the dosage of hydrochlorothiazide is from about 12.5 mg to about 50 mg. In some such embodiments, the dosage of hydrochlorothiazide is about 12.5 mg, about 25 mg, or about 50 mg. In some embodiments, the first pharmaceutical composition of the capsule is the dosage of about 81 mg of aspirin, the second pharmaceutical composition of the capsule is the dosage of about 80 mg of atorvastatin, the third pharmaceutical composition of the capsule is the dosage of about 10 mg of ezetimibe, the fourth pharmaceutical composition of the capsule is the dosage of about 150 mg of metoprolol, the fifth pharmaceutical composition of the capsule is the dosage of about 5 mg of lisinopril, and the sixth pharmaceutical composition of the capsule is the dosage of about 12.5 mg of hydrochlorothiazide.

In some embodiments, the first pharmaceutical composition is aspirin. In some such embodiments, the dosage of aspirin is from about 50 mg to about 1,000 mg. In some such embodiments, the dosage of aspirin is about 81 mg, about 150 mg, about 300 mg, about 600 mg, or about 800 mg. In some embodiments, the second pharmaceutical composition is atorvastatin. In some such embodiments, the dosage of atorvastatin is of from about 10 mg to about 80 mg. In some embodiments, the dosage of atorvastatin is about 10 mg, about 20 mg, about 40 mg, or about 80 mg. In some embodiments, the third pharmaceutical composition is ezetimibe. In some such embodiments, the dosage of ezetimibe is about 10 mg. In some embodiments, the fourth pharmaceutical composition is metoprolol succinate. In some such embodiments, the dosage of metoprolol succinate is of from about 25 mg to about 200 mg. In some such embodiments, the dosage of metoprolol succinate is about 25 mg, about 50 mg, about 100 mg, or about 200 mg. In some embodiments, the fifth pharmaceutical composition is enalapril. In some such embodiments, the dosage of enalapril is of from about 2.5 mg to about 40 mg. In some embodiments, the dosage of enalapril is about 2.5 mg, about 5 mg, about 10 mg, about 20 mg. In some embodiments, the sixth pharmaceutical composition is spironolactone. In some such embodiments, the dosage of spironolactone is from about 12.5 mg to about 100 mg. In some such embodiments, the dosage of spironolactone is about 25 mg, about 50 mg, or about 100 mg. In some embodiments, the seventh pharmaceutical composition is torsemide. In some such embodiments, the dosage of torsemide is from about 5 mg to about 100 mg. In some such embodiments, the dosage of torsemide is about 5 mg, about 10 mg, about 20 mg, or about 100 mg. In some embodiments, the first pharmaceutical composition of the capsule is the dosage of about 81 mg of aspirin, the second pharmaceutical composition of the capsule is the dosage of about 40 mg of atorvastatin, the third pharmaceutical composition of the capsule is the dosage of about 10 mg of ezetimibe, the fourth pharmaceutical composition of the capsule is the dosage of about 200 mg of metoprolol, the fifth pharmaceutical composition of the capsule is the dosage of about 20 mg of enalapril, the sixth pharmaceutical composition of the capsule is the dosage of about 12.5 mg spironolactone, and the seventh pharmaceutical composition of the capsule is the dosage of about 120 mg of torsemide.

In some embodiments, the first pharmaceutical composition is prasugrel. In some such embodiments, the dosage of prasugrel is from about 5 mg to about 10 mg. In some such embodiments, the dosage of prasugrel is about 5 mg, or about 10 mg. In some embodiments, the second pharmaceutical composition is atorvastatin. In some such embodiments, the dosage of atorvastatin is of from about 10 mg to about 80 mg. In some embodiments, the dosage of atorvastatin is about 10 mg, about 20 mg, about 40 mg, or about 80 mg. In some embodiments, the third pharmaceutical composition is ezetimibe. In some such embodiments, the dosage of ezetimibe is about 10 mg. In some embodiments, the fourth pharmaceutical composition is metoprolol succinate. In some such embodiments, the dosage of metoprolol succinate is of from about 25 mg to about 200 mg. In some such embodiments, the dosage of metoprolol succinate is about 25 mg, about 50 mg, about 100 mg, or about 200 mg. In some embodiments, the fifth pharmaceutical composition is lisinopril. In some such embodiments, the dosage of lisinopril is of from about 2.5 mg to about 40 mg. In some embodiments, the dosage of lisinopril is about 2.5 mg, about 5 mg, about 10 mg, about 20 mg, about 30 mg, or about 40 mg. In some embodiments, the sixth pharmaceutical composition is hydrochlorothiazide. In some such embodiments, the dosage of hydrochlorothiazide is from about 12.5 mg to about 50 mg. In some such embodiments, the dosage of hydrochlorothiazide is about 12.5 mg, about 25 mg, or about 50 mg. In some embodiments, the first pharmaceutical composition of the capsule is the dosage of about 5 mg or about 10 mg of prasugrel, the second pharmaceutical composition of the capsule is the dosage of about 80 mg of atorvastatin, the third pharmaceutical composition of the capsule is the dosage of about 10 mg of ezetimibe, the fourth pharmaceutical composition of the capsule is the dosage of about 150 mg of metoprolol, the fifth pharmaceutical composition of the capsule is the dosage of about 5 mg of lisinopril, and the sixth pharmaceutical composition of the capsule is the dosage of about 12.5 mg of hydrochlorothiazide.

In some embodiments, the first pharmaceutical composition is prasugrel. In some such embodiments, the dosage of prasugrel is from about 5 mg to about 10 mg. In some such embodiments, the dosage of prasugrel is about 5 mg, or about 10 mg. In some embodiments, the second pharmaceutical composition is atorvastatin. In some such embodiments, the dosage of atorvastatin is of from about 10 mg to about 80 mg. In some embodiments, the dosage of atorvastatin is about 10 mg, about 20 mg, about 40 mg, or about 80 mg. In some embodiments, the third pharmaceutical composition is ezetimibe. In some such embodiments, the dosage of ezetimibe is about 10 mg. In some embodiments, the fourth pharmaceutical composition is metoprolol succinate. In some such embodiments, the dosage of metoprolol succinate is of from about 25 mg to about 200 mg. In some such embodiments, the dosage of metoprolol succinate is about 25 mg, about 50 mg, about 100 mg, or about 200 mg. In some embodiments, the fifth pharmaceutical composition is enalapril. In some such embodiments, the dosage of enalapril is of from about 2.5 mg to about 40 mg. In some embodiments, the dosage of enalapril is about 2.5 mg, about 5 mg, about 10 mg, or about 20 mg. In some embodiments, the sixth pharmaceutical composition is spironolactone. In some such embodiments, the dosage of spironolactone is from about 12.5 mg to about 100 mg. In some such embodiments, the dosage of spironolactone is about 25 mg, about 50 mg, or about 100 mg. In some embodiments, the seventh pharmaceutical composition is torsemide. In some such embodiments, the dosage of torsemide is from about 5 mg to about 100 mg. In some such embodiments, the dosage of torsemide is about 5 mg, about 10 mg, about 20 mg, or about 100 mg. In some embodiments, the first pharmaceutical composition of the capsule is the dosage of about 5 mg or about 10 mg of prasugrel, the second pharmaceutical composition of the capsule is the dosage of about 40 mg of atorvastatin, the third pharmaceutical composition of the capsule is the dosage of about 10 mg of ezetimibe, the fourth pharmaceutical composition of the capsule is the dosage of about 200 mg of metoprolol, the fifth pharmaceutical composition of the capsule is the dosage of about 20 mg of enalapril, the sixth pharmaceutical composition of the capsule is the dosage of about 12.5 mg spironolactone, and the seventh pharmaceutical composition of the capsule is the dosage of about 120 mg of torsemide.

In some embodiments, the first pharmaceutical composition is lisinopril. In some such embodiments, the dosage of lisinopril is of from about 2.5 mg to about 40 mg. In some embodiments, the dosage of lisinopril is about 2.5 mg, about 5 mg, about 10 mg, about 20 mg, about 30 mg, or about 40 mg. In some embodiments, the second pharmaceutical composition is carvedilol phosphate. In some such embodiments, the dosage of carvedilol phosphate is of from about 10 mg to about 80 mg. In some such embodiments, the dosage of carvedilol phosphate is about 10 mg, about 20 mg, about 40 mg, or about 80 mg. In some embodiments, the third pharmaceutical composition is amlodipine. In some such embodiments, the dosage of amlodipine is of from about 1 mg to about 10 mg. In some such embodiments, the dosage of amlodipine is about 2.5 mg, about 5 mg, or about 10 mg. In some embodiments, the fourth pharmaceutical composition is spironolactone. In some such embodiments, the dosage of spironolactone is from about 12.5 mg to about 100 mg. In some such embodiments, the dosage of spironolactone is about 25 mg, about 50 mg, or about 100 mg. In some embodiments, the fifth pharmaceutical composition is furosemide. In some such embodiments, the dosage of furosemide is of from about 20 mg to about 200 mg. In some embodiments, the dosage of furosemide is about 20 mg, about 40 mg, or about 80 mg. In some embodiments, the first pharmaceutical composition of the capsule is the dosage of about 40 mg of lisinopril, the second pharmaceutical composition of the capsule is the dosage of about 50 mg of carvedilol, the third pharmaceutical composition of the capsule is the dosage of about 5 mg of amlodipine, the fourth pharmaceutical composition of the capsule is the dosage of about 25 mg of spironolactone n, and the fifth pharmaceutical composition of the capsule is the dosage of about 200 mg of furosemide.

In some embodiments, the first pharmaceutical composition is prasugrel. In some such embodiments, the dosage of prasugrel is from about 5 mg to about 10 mg. In some such embodiments, the dosage of prasugrel is about 5 mg or about 10 mg. In some embodiments, the second pharmaceutical composition is atorvastatin. In some such embodiments, the dosage of atorvastatin is of from about 10 mg to about 80 mg. In some embodiments, the dosage of atorvastatin is about 10 mg, about 20 mg, about 40 mg, or about 80 mg. In some embodiments, the third pharmaceutical composition is ezetimibe. In some such embodiments, the dosage of ezetimibe is about 10 mg. In some embodiments, the fourth pharmaceutical composition is metoprolol succinate. In some such embodiments, the dosage of metoprolol succinate is of from about 25 mg to about 200 mg. In some such embodiments, the dosage of metoprolol succinate is about 25 mg, about 50 mg, about 100 mg, or about 200 mg. In some embodiments, the fifth pharmaceutical composition is valsartan. In some such embodiments, the dosage of fifth is of from about 20 mg to about 320 mg. In some embodiments, the dosage of fifth is about 40 mg, about 80 mg, or about 160 mg. In some embodiments, the sixth pharmaceutical composition is spironolactone. In some such embodiments, the dosage of spironolactone is from about 12.5 mg to about 100 mg. In some such embodiments, the dosage of spironolactone is about 25 mg, about 50 mg, or about 100 mg. In some embodiments, the seventh pharmaceutical composition is furosemide. In some such embodiments, the dosage of furosemide is of from about 20 to about 200 mg. In some embodiments, the dosage of furosemide is about 20 mg, about 40 mg, or about 80 mg. In some embodiments, an eighth pharmaceutical composition is amlodipine. In some such embodiments, a dosage of amlodipine is of from about 1 mg to about 10 mg. In some such embodiments, the dosage of amlodipine is about 2.5 mg, about 5 mg, or about 10 mg. In some embodiments, the first pharmaceutical composition of the capsule is the dosage of about 5 mg or about 10 mg of prasugrel, the second pharmaceutical composition of the capsule is the dosage of about 40 mg of atorvastatin, the third pharmaceutical composition of the capsule is the dosage of about 10 mg of ezetimibe, the fourth pharmaceutical composition of the capsule is the dosage of about 200 mg of metoprolol, the fifth pharmaceutical composition of the capsule is the dosage of about 20 mg of valsartan, the sixth pharmaceutical composition of the capsule is the dosage of about 12.5 mg spironolactone, the seventh pharmaceutical composition of the capsule is the dosage of about 160 mg of furosemide, and the eighth pharmaceutical composition of the capsule is the dosage of about 5 mg of amlodipine.

In some embodiments, the first unique interior volume includes a CCB pharmaceutical composition, the second unique interior volume includes an ACEi pharmaceutical composition, and the third unique interior volume includes a thiazide pharmaceutical composition. In some embodiments, a member selected from the first unique interior volume and the second unique interior volume, the second unique interior volume and the third unique interior volume, and the first unique interior volume and the third unique interior volume do not include a class of pharmaceutical compositions that is the same as the pharmaceutical composition in another unique interior volume.

In some embodiments, the first unique interior volume includes amlodipine (e.g., about 10 mg of amlodipine), the second unique interior volume includes the ACEi pharmaceutical composition (e.g., about 5 mg of lisinopril), and the third unique interior volume includes the thiazide pharmaceutical composition (e.g., about 25 mg of hydrochlorothiazide).

In some embodiments, the first unique interior volume includes the CCB pharmaceutical composition (e.g., about 10 mg of amlodipine), the second unique interior volume includes lisinopril (e.g., about 5 mg of lisinopril), and the third unique interior volume includes the thiazide pharmaceutical composition (e.g., about 25 mg of hydrochlorothiazide).

In some embodiments, the first unique interior volume includes the CCB pharmaceutical composition (e.g., about 10 mg amlodipine), the second unique interior volume includes the ACEi pharmaceutical composition (e.g., about 5 mg of lisinopril), and the third unique interior volume includes hydrochlorothiazide (e.g., about 25 mg of hydrochlorothiazide).

Furthermore, in some embodiments, the first unique interior volume includes the CCB pharmaceutical composition, the second unique interior volume includes an ARB pharmaceutical composition, and the third unique interior volume includes the thiazide pharmaceutical composition. In some embodiments, a member selected from the first unique interior volume and the second unique interior volume, the second unique interior volume and the third unique interior volume, and the first unique interior volume and the third unique interior volume do not include a class of pharmaceutical compositions that is the same as in another unique interior volume.

In some embodiments, the first unique interior volume includes amlodipine (e.g., about 10 mg of amlodipine), the second unique interior volume includes the ARB pharmaceutical composition (e.g., about 50 mg of losartan, about 100 mg of losartan, about 320 mg of valsartan), and the third unique interior volume includes the thiazide pharmaceutical composition (e.g., about 12.5 mg of chlorthalidone, about 25 mg of hydrochlorothiazide).

In some embodiments, the first unique interior volume includes the CCB pharmaceutical composition (e.g., about 10 mg of amlodipine), the second unique interior volume includes losartan (e.g., about 50 mg of losartan, about 100 mg of losartan), and the third unique interior volume includes the thiazide pharmaceutical composition (e.g., about 12.5 mg of chlorthalidone, about 25 mg of hydrochlorothiazide).

In some embodiments, the first unique interior volume includes the CCB pharmaceutical composition (e.g., about 10 mg of amlodipine), the second unique interior volume includes valsartan (e.g., about 320 mg of valsartan), and the third unique interior volume includes the thiazide pharmaceutical composition (e.g., about 12.5 mg of chlorthalidone, about 25 mg of hydrochlorothiazide).

In some embodiments, the first unique interior volume includes the CCB pharmaceutical composition (e.g., about 10 mg of amlodipine), the second unique interior volume includes the ARB pharmaceutical composition (e.g., about 50 mg of losartan, about 100 mg of losartan, about 320 mg of valsartan), and the third unique interior volume includes hydrochlorothiazide (e.g., about 25 mg of hydrochlorothiazide).

In some embodiments, the first unique interior volume includes the CCB pharmaceutical composition (e.g., about 10 mg of amlodipine), the second unique interior volume includes the ARB pharmaceutical composition (e.g., about 50 mg of losartan, about 100 mg of losartan, about 320 mg of valsartan), and the third unique interior volume includes chlorthalidone (e.g., about 12.5 mg of chlorthalidone).

In some embodiments, the first unique interior volume includes the ACEi pharmaceutical composition, the second unique interior volume includes the thiazide pharmaceutical composition, and the second unique interior volume includes the thiazide pharmaceutical composition. In some embodiments, a member selected from the first unique interior volume and the second unique interior volume, the second unique interior volume and the third unique interior volume, and the first unique interior volume and the third unique interior volume do not include a class of pharmaceutical compositions that is the same as in another unique interior volume.

In some embodiments, the first unique interior volume includes lisinopril (e.g., about 40 mg of lisinopril), the second unique interior volume includes the thiazide pharmaceutical composition (e.g., about 25 mg of hydrochlorothiazide, about 50 mg of hydrochlorothiazide), and the third unique interior volume includes the BB pharmaceutical composition (e.g., about 100 mg of metoprolol succinate, about 5 mg of bisoprolol).

In some embodiments, the first unique interior volume includes ramipril (e.g., 10 mg of ramipril), the second unique interior volume includes the thiazide pharmaceutical composition (e.g., about 25 mg of hydrochlorothiazide, about 50 mg of hydrochlorothiazide), and the third unique interior volume includes the BB pharmaceutical composition (e.g., about 100 mg of metoprolol succinate, about 5 mg of bisoprolol).

In some embodiments, the first unique interior volume includes the ACEi pharmaceutical composition (e.g., about mg of lisinopril, about 10 mg of ramipril), the second unique interior volume includes hydrochlorothiazide (e.g., about 25 mg of hydrochlorothiazide, about 50 mg of hydrochlorothiazide), and the third unique interior volume includes the BB pharmaceutical composition (e.g., about 100 mg of metoprolol succinate, about 5 mg of bisoprolol).

In some embodiments, the first unique interior volume includes the ACEi pharmaceutical composition (e.g., about 40 mg of lisinopril, about 10 mg of ramipril), the second unique interior volume includes the thiazide pharmaceutical composition (e.g., about 25 mg of hydrochlorothiazide, about 50 mg of hydrochlorothiazide), and the third unique interior volume includes metoprolol succinate (e.g., about 100 mg of metoprolol succinate).

In some embodiments, the first unique interior volume includes the ACEi pharmaceutical composition (e.g., about 40 mg of lisinopril, about 10 mg of ramipril), the second unique interior volume includes the thiazide pharmaceutical composition (e.g., about 25 mg of hydrochlorothiazide, about 50 mg of hydrochlorothiazide), and the third unique interior volume includes bisoprolol (e.g., about 5 mg of bisoprolol).

Furthermore, in some embodiments, the first unique interior volume includes the CCB pharmaceutical composition, the second unique interior volume includes the ACEi pharmaceutical composition, the third unique interior volume includes the thiazide pharmaceutical composition, and a fourth unique interior volume includes the K-sparing diuretic pharmaceutical composition. In some embodiments, a member selected from: the first unique interior volume, the second unique interior volume, and the third unique interior volume; the second unique interior volume, the third unique interior volume, the fourth unique interior volume; and the first unique interior volume, the third unique interior volume, and the fourth unique interior volume do not include a class of pharmaceutical compositions that is the same as in another unique interior volume.

In some embodiments, the first unique interior volume includes amlodipine (e.g., about 10 mg of amlodipine), the second unique interior volume includes the ACEi pharmaceutical composition (e.g., about 40 mg of lisinopril), the third unique interior volume includes the thiazide pharmaceutical composition (e.g., about 25 mg of hydrochlorothiazide), and the fourth unique interior volume includes the K-sparing diuretic pharmaceutical composition (e.g., about 12.5 mg of spironolactone).

In some embodiments, the first unique interior volume includes the CCB pharmaceutical composition (e.g., about 10 mg of amlodipine), the second unique interior volume includes lisinopril (e.g., about 40 mg of lisinopril), the third unique interior volume includes the thiazide pharmaceutical composition (e.g., about 25 mg of hydrochlorothiazide), and the fourth unique interior volume includes the K-sparing diuretic pharmaceutical composition (e.g., about 12.5 mg of spironolactone).

In some embodiments, the first unique interior volume includes the CCB pharmaceutical composition (e.g., about 10 mg of amlodipine), the second unique interior volume includes the ACEi pharmaceutical composition (e.g., about 40 mg of lisinopril), the third unique interior volume includes hydrochlorothiazide (e.g., about 25 mg of hydrochlorothiazide), and the fourth unique interior volume includes the K-sparing diuretic pharmaceutical composition (e.g., about 12.5 mg of spironolactone).

In some embodiments, the first unique interior volume includes the CCB pharmaceutical (e.g., about 10 mg of amlodipine), the second unique interior volume includes the ACEi pharmaceutical composition (e.g., about 40 mg of lisinopril), the third unique interior volume includes the thiazide pharmaceutical composition (e.g., about 25 mg of hydrochlorothiazide), and the fourth unique interior volume includes spironolactone (e.g., about 12.5 mg of spironolactone).

Additionally, in some embodiments, the first unique interior volume includes the BB pharmaceutical composition, the second unique interior volume includes the ACEi pharmaceutical composition, the third unique interior volume includes the anti-platelet pharmaceutical composition, and the fourth unique interior volume includes the lipid-lowering pharmaceutical composition. In some embodiments, a member selected from: the first unique interior volume, the second unique interior volume, and the third unique interior volume; the second unique interior volume, the third unique interior volume, the fourth unique interior volume; and the first unique interior volume, the third unique interior volume, and the fourth unique interior volume do not include a class of pharmaceutical compositions that is the same as in another unique interior volume.

In some embodiments, the first unique interior volume includes metoprolol succinate (e.g., about 150 mg of metoprolol succinate), the second unique interior volume includes the ACEi pharmaceutical composition (e.g., about 40 mg of lisinopril), the third unique interior volume includes the anti-platelet pharmaceutical composition (e.g., about 81 mg of aspirin, about 5 mg of prasugrel, about 10 mg of prasugrel), and the fourth unique interior volume includes the lipid-lowering pharmaceutical composition (e.g., about 80 mg of atorvastatin).

In some embodiments, the first unique interior volume includes the BB pharmaceutical composition (e.g., about 150 mg of metoprolol succinate), the second unique interior volume includes lisinopril (e.g., about 40 mg of lisinopril), the third unique interior volume includes the anti-platelet pharmaceutical composition (e.g., about 81 mg of aspirin, about 5 mg of prasugrel, about 10 mg of prasugrel), and the fourth unique interior volume includes the lipid-lowering pharmaceutical composition (e.g., about 80 mg of atorvastatin).

In some embodiments, the first unique interior volume includes the BB pharmaceutical composition (e.g., about 150 mg of metoprolol succinate), the second unique interior volume includes the ACEi pharmaceutical composition (e.g., about 40 mg of lisinopril), the third unique interior volume includes aspirin (e.g., about 81 mg of aspirin), and the fourth unique interior volume includes the lipid-lowering pharmaceutical composition (e.g., about 80 mg of atorvastatin).

In some embodiments, the first unique interior volume includes the BB pharmaceutical composition (e.g., about 150 mg of metoprolol succinate), the second unique interior volume includes the ACEi pharmaceutical composition (e.g., about 40 mg of lisinopril), the third unique interior volume includes prasugrel (e.g., about 5 mg of prasugrel, about 10 mg of prasugrel), and the fourth unique interior volume includes the lipid-lowering pharmaceutical composition (e.g., about 80 mg of atorvastatin).

In some embodiments, the first unique interior volume includes the BB pharmaceutical composition (e.g., about 150 mg of metoprolol succinate), the second unique interior volume includes the ACEi pharmaceutical composition (e.g., about 40 mg of lisinopril), the third unique interior volume includes the anti-platelet pharmaceutical composition (e.g., about 81 mg of aspirin, about 5 mg of prasugrel, 10 mg of prasugrel), and the fourth unique interior volume includes atorvastatin (e.g., about 80 mg of atorvastatin).

Furthermore, in some embodiments, the first unique interior volume includes the CCB pharmaceutical composition, the second unique interior volume includes the ARB pharmaceutical composition, the third unique interior volume includes the anti-platelet pharmaceutical composition, the fourth unique interior volume includes a first lipid-lowering pharmaceutical composition, and the fifth unique interior volume includes a second lipid-lowering pharmaceutical composition. In some embodiments, a member selected from: the first unique interior volume, the second unique interior volume, the third unique interior volume, and the fourth interior volume; the first unique interior volume, the second unique interior volume, the third unique interior volume, and the fifth unique interior volume; the first unique interior volume, the second unique interior volume, the fourth unique interior volume, and the fifth unique interior volume; the second unique interior volume, the third unique interior volume, the fourth unique interior volume, and the fifth unique interior do not include a class of pharmaceutical compositions that is the same as in another unique interior volume.

In some embodiments, the first unique interior volume includes diltiazem (e.g., about 180 mg of diltiazem), the second unique interior volume includes the ACEi pharmaceutical composition (e.g., about 80 mg of valsartan), the third unique interior volume includes the anti-platelet pharmaceutical composition (e.g., about 81 mg of aspirin, about 5 mg of prasugrel, about 10 mg of prasugrel), the fourth unique interior volume includes the first lipid-lowering pharmaceutical composition (e.g., about 20 mg of pravastatin), and the fifth unique interior volume includes the second lipid-lowering pharmaceutical composition (e.g., about 10 mg of ezetimibe).

In some embodiments, the first unique interior volume includes the CCB pharmaceutical composition (e.g., about 180 mg of diltiazem), the second unique interior volume includes valsartan (e.g., about 80 mg of valsartan), the third unique interior volume includes the anti-platelet pharmaceutical composition (e.g., about 81 mg of aspirin, about 5 mg of prasugrel, about 10 mg of prasugrel), the fourth unique interior volume includes the first lipid-lowering pharmaceutical composition (e.g., about 20 mg of pravastatin), and the fifth unique interior volume includes the second lipid-lowering pharmaceutical composition (e.g., about 10 mg of ezetimibe).

In some embodiments, the first unique interior volume includes the CCB pharmaceutical composition (e.g., about 180 mg of diltiazem), the second unique interior volume includes the ACEi pharmaceutical composition (e.g., about 80 mg of valsartan), the third unique interior volume includes aspirin (e.g., about 81 mg of aspirin), the fourth unique interior volume includes the first lipid-lowering pharmaceutical composition (e.g., about 20 mg of pravastatin), and the fifth unique interior volume includes the second lipid-lowering pharmaceutical composition (e.g., about 10 mg of ezetimibe).

In some embodiments, the first unique interior volume includes the CCB pharmaceutical composition (e.g., about 180 mg of diltiazem), the second unique interior volume includes the ACEi pharmaceutical composition (e.g., about 80 mg of valsartan), the third unique interior volume includes prasugrel (e.g., about 5 mg of prasugrel, about 10 mg of prasugrel), the fourth unique interior volume includes the first lipid-lowering pharmaceutical composition (e.g., about 20 mg of pravastatin), and the fifth unique interior volume includes the second lipid-lowering pharmaceutical composition (e.g., about 10 mg of ezetimibe).

In some embodiments, the first unique interior volume includes the CCB pharmaceutical composition (e.g., about 180 mg of diltiazem), the second unique interior volume includes the ACEi pharmaceutical composition (e.g., about 80 mg of valsartan), the third unique interior volume includes the anti-platelet pharmaceutical composition (e.g., about 81 mg of aspirin, about 5 mg of prasugrel, about 10 mg of prasugrel), the fourth unique interior volume includes pravastatin (e.g., about 20 mg of pravastatin), and the fifth unique interior volume includes the second lipid-lowering pharmaceutical composition (e.g., about 10 mg of ezetimibe).

In some embodiments, the first unique interior volume includes the CCB pharmaceutical composition (e.g., about 120 mg of diltiazem, about 180 mg of diltiazem), the second unique interior volume includes the BB pharmaceutical composition (e.g., about 100 mg of metoprolol succinate, about 200 mg of metoprolol succinate), the third unique interior volume includes the anti-platelet pharmaceutical composition (e.g., about 81 mg of aspirin, about 5 mg of prasugrel, about 10 mg of prasugrel), the fourth unique interior volume includes the first lipid-lowering pharmaceutical composition (e.g., about 20 mg of pravastatin), and the fifth unique interior volume includes ezetimibe (e.g., about 10 mg of ezetimibe).

Furthermore, in some embodiments, the first unique interior volume includes the CCB pharmaceutical composition, the second unique interior volume includes the BB pharmaceutical composition, the third unique interior volume includes the ACEi pharmaceutical composition, the fourth unique interior volume includes the anti-platelet pharmaceutical composition, the fifth unique interior volume includes the lipid-lowering pharmaceutical composition, and the sixth unique interior volume includes the anti-anginal pharmaceutical composition. In some embodiments, a member selected from: the first unique interior volume, the second unique interior volume, the third unique interior volume, the fourth interior volume, and the fifth unique interior volume; the first unique interior volume, the second unique interior volume, the third unique interior volume, the fourth unique interior volume, and the sixth unique interior volume; the first unique interior volume, the second unique interior volume, the third unique interior volume, the fifth unique interior volume, and the sixth unique interior volume; the first unique interior volume, the second unique interior volume, the fourth unique interior volume, the fifth unique interior volume, and the sixth unique interior volume; the first unique interior volume, the third unique interior volume, the fourth unique interior volume, the fifth unique interior volume, and the sixth unique interior volume; the second unique interior volume, the third unique interior volume, the fourth unique interior volume, the fifth unique interior volume, and the sixth unique interior volume do not include a class of pharmaceutical compositions that is the same as in another unique interior volume.

In some embodiments, the first unique interior volume includes the CCB pharmaceutical composition (e.g., about 120 mg of diltiazem, about 180 mg of diltiazem), the second unique interior volume includes the BB pharmaceutical composition (e.g., about 100 mg of metoprolol succinate, about 200 mg of metoprolol succinate), the third unique interior volume includes the ACEi pharmaceutical composition (e.g., about 20 mg lisinopril), the fourth unique interior volume includes the anti-platelet pharmaceutical composition (e.g., about 81 mg of aspirin, about 5 mg of prasugrel, about 10 mg of prasugrel), the fifth unique interior volume includes the lipid-lowering pharmaceutical composition (e.g., about 80 mg of atorvastatin), and the sixth unique interior volume includes the anti-anginal pharmaceutical composition (e.g., about 10 mg of isosorbide mononitrate).

In some embodiments, the first unique interior volume includes the CCB pharmaceutical composition (e.g., about 120 mg of diltiazem, about 180 mg of diltiazem), the second unique interior volume includes the BB pharmaceutical composition (e.g., about 100 mg of metoprolol succinate, about 200 mg of metoprolol succinate), the third unique interior volume includes the ACEi pharmaceutical composition (e.g., about 20 mg lisinopril), the fourth unique interior volume includes the anti-platelet pharmaceutical composition (e.g., about 81 mg of aspirin, about 5 mg of prasugrel, about 10 mg of prasugrel), the fifth unique interior volume includes the lipid-lowering pharmaceutical composition (e.g., about 80 mg of atorvastatin), and the sixth unique interior volume includes the anti-anginal pharmaceutical composition (e.g., about 10 mg of isosorbide mononitrate).

In some embodiments, the first unique interior volume includes diltiazem (e.g., about 120 mg of diltiazem, about 180 mg of diltiazem), the second unique interior volume includes the BB pharmaceutical composition (e.g., about 100 mg of metoprolol succinate, about 200 mg of metoprolol succinate), the third unique interior volume includes the ACEi pharmaceutical composition (e.g., about 20 mg lisinopril), the fourth unique interior volume includes the anti-platelet pharmaceutical composition (e.g., about 81 mg of aspirin, about 5 mg of prasugrel, about 10 mg of prasugrel), the fifth unique interior volume includes the lipid-lowering pharmaceutical composition (e.g., about 80 mg of atorvastatin), and the sixth unique interior volume includes the anti-anginal pharmaceutical composition (e.g., about 10 mg of isosorbide mononitrate).

In some embodiments, the first unique interior volume includes the CCB pharmaceutical composition (e.g., about 120 mg of diltiazem, about 180 mg of diltiazem), the second unique interior volume includes metoprolol succinate (e.g., about 100 mg of metoprolol succinate, about 200 mg of metoprolol succinate), the third unique interior volume includes the ACEi pharmaceutical composition (e.g., about 20 mg lisinopril), the fourth unique interior volume includes the anti-platelet pharmaceutical composition (e.g., about 81 mg of aspirin, about 5 mg of prasugrel, about 10 mg of prasugrel), the fifth unique interior volume includes the lipid-lowering pharmaceutical composition (e.g., about 80 mg of atorvastatin), and the sixth unique interior volume includes the anti-anginal pharmaceutical composition (e.g., about 10 mg of isosorbide mononitrate).

In some embodiments, the first unique interior volume includes the CCB pharmaceutical composition (e.g., about 120 mg of diltiazem, about 180 mg of diltiazem), the second unique interior volume includes the BB pharmaceutical composition (e.g., about 100 mg of metoprolol succinate, about 200 mg of metoprolol succinate), the third unique interior volume includes lisinopril (e.g., about 20 mg lisinopril), the fourth unique interior volume includes the anti-platelet pharmaceutical composition (e.g., about 81 mg of aspirin, about 5 mg of prasugrel, about 10 mg of prasugrel), the fifth unique interior volume includes the lipid-lowering pharmaceutical composition (e.g., about 80 mg of atorvastatin), and the sixth unique interior volume includes the anti-anginal pharmaceutical composition (e.g., about 10 mg of isosorbide mononitrate).

In some embodiments, the first unique interior volume includes the CCB pharmaceutical composition (e.g., about 120 mg of diltiazem, about 180 mg of diltiazem), the second unique interior volume includes the BB pharmaceutical composition (e.g., about 100 mg of metoprolol succinate, about 200 mg of metoprolol succinate), the third unique interior volume includes the ACEi pharmaceutical composition (e.g., about 20 mg lisinopril), the fourth unique interior volume includes aspirin (e.g., about 81 mg of aspirin), the fifth unique interior volume includes the lipid-lowering pharmaceutical composition (e.g., about 80 mg of atorvastatin), and the sixth unique interior volume includes the anti-anginal pharmaceutical composition (e.g., about 10 mg of isosorbide mononitrate).

In some embodiments, the first unique interior volume includes the CCB pharmaceutical composition (e.g., about 120 mg of diltiazem, about 180 mg of diltiazem), the second unique interior volume includes the BB pharmaceutical composition (e.g., about 100 mg of metoprolol succinate, about 200 mg of metoprolol succinate), the third unique interior volume includes the ACEi pharmaceutical composition (e.g., about 20 mg lisinopril), the fourth unique interior volume includes prasugrel (e.g., about 5 mg of prasugrel, about 10 mg of prasugrel), the fifth unique interior volume includes the lipid-lowering pharmaceutical composition (e.g., about 80 mg of atorvastatin), and the sixth unique interior volume includes the anti-anginal pharmaceutical composition (e.g., about 10 mg of isosorbide mononitrate).

In some embodiments, the first unique interior volume includes the CCB pharmaceutical composition (e.g., about 120 mg of diltiazem, about 180 mg of diltiazem), the second unique interior volume includes the BB pharmaceutical composition (e.g., about 100 mg of metoprolol succinate, about 200 mg of metoprolol succinate), the third unique interior volume includes the ACEi pharmaceutical composition (e.g., about 20 mg lisinopril), the fourth unique interior volume includes the anti-platelet pharmaceutical composition (e.g., about 81 mg of aspirin, about 5 mg of prasugrel, about 10 mg of prasugrel), the fifth unique interior volume includes the atorvastatin (e.g., about 80 mg of atorvastatin), and the sixth unique interior volume includes the anti-anginal pharmaceutical composition (e.g., about 10 mg of isosorbide mononitrate).

In some embodiments, the first unique interior volume includes the CCB pharmaceutical composition (e.g., about 120 mg of diltiazem, about 180 mg of diltiazem), the second unique interior volume includes the BB pharmaceutical composition (e.g., about 100 mg of metoprolol succinate, about 200 mg of metoprolol succinate), the third unique interior volume includes the ACEi pharmaceutical composition (e.g., about 20 mg lisinopril), the fourth unique interior volume includes the anti-platelet pharmaceutical composition (e.g., about 81 mg of aspirin, about 5 mg of prasugrel, about 10 mg of prasugrel), the fifth unique interior volume includes the lipid-lowering pharmaceutical composition (e.g., about 80 mg of atorvastatin), and the sixth unique interior volume includes isosorbide mononitrate (e.g., about 10 mg of isosorbide mononitrate).

Furthermore, in some embodiments, the first unique interior volume includes the CCB pharmaceutical composition, the second unique interior volume includes the BB pharmaceutical composition, the third unique interior volume includes the ARB pharmaceutical composition, the fourth unique interior volume includes the anti-platelet pharmaceutical composition, the fifth unique interior volume includes a first lipid-lowering pharmaceutical composition, and the sixth unique interior volume includes a second lipid-lowering pharmaceutical composition. In some embodiments, a member selected from: the first unique interior volume, the second unique interior volume, the third unique interior volume, the fourth interior volume, and the fifth unique interior volume; the first unique interior volume, the second unique interior volume, the third unique interior volume, the fourth unique interior volume, and the sixth unique interior volume; the first unique interior volume, the second unique interior volume, the third unique interior volume, the fifth unique interior volume, and the sixth unique interior volume; the first unique interior volume, the second unique interior volume, the fourth unique interior volume, the fifth unique interior volume, and the sixth unique interior volume; the first unique interior volume, the third unique interior volume, the fourth unique interior volume, the fifth unique interior volume, and the sixth unique interior volume; the second unique interior volume, the third unique interior volume, the fourth unique interior volume, the fifth unique interior volume, and the sixth unique interior volume do not include a class of pharmaceutical compositions that is the same as in another unique interior volume.

In some embodiments, the first unique interior volume includes diltiazem (e.g., about 120 mg of diltiazem), the second unique interior volume includes the BB pharmaceutical composition (e.g., about 25 mg of carvedilol phosphate), the third unique interior volume includes the ARB pharmaceutical composition (e.g., about 25 mg losartan), the fourth unique interior volume includes the anti-platelet pharmaceutical composition (e.g., about 81 mg of aspirin, about 5 mg of prasugrel, about 10 mg of prasugrel), the fifth unique interior volume includes the first lipid-lowering pharmaceutical composition (e.g., about 40 mg of rosuvastatin), and the sixth unique interior volume includes the second lipid-lowering pharmaceutical composition (e.g., about 10 mg of ezetimibe).

In some embodiments, the first unique interior volume includes the CCB pharmaceutical composition (e.g., about 120 mg of diltiazem), the second unique interior volume includes carvedilol phosphate (e.g., about 25 mg of carvedilol phosphate), the third unique interior volume includes the ARB pharmaceutical composition (e.g., about 25 mg losartan), the fourth unique interior volume includes the anti-platelet pharmaceutical composition (e.g., about 81 mg of aspirin, about 5 mg of prasugrel, about 10 mg of prasugrel), the fifth unique interior volume includes the first lipid-lowering pharmaceutical composition (e.g., about 40 mg of rosuvastatin), and the sixth unique interior volume includes the second lipid-lowering pharmaceutical composition (e.g., about 10 mg of ezetimibe).

In some embodiments, the first unique interior volume includes the CCB pharmaceutical composition (e.g., about 120 mg of diltiazem), the second unique interior volume includes the BB pharmaceutical composition (e.g., about 25 mg of carvedilol phosphate), the third unique interior volume includes the losartan (e.g., about 25 mg losartan), the fourth unique interior volume includes the anti-platelet pharmaceutical composition (e.g., about 81 mg of aspirin), the fifth unique interior volume includes the first lipid-lowering pharmaceutical composition (e.g., about 40 mg of rosuvastatin), and the sixth unique interior volume includes the second lipid-lowering pharmaceutical composition (e.g., about 10 mg of ezetimibe).

In some embodiments, the first unique interior volume includes the CCB pharmaceutical composition (e.g., about 120 mg of diltiazem), the second unique interior volume includes the BB pharmaceutical composition (e.g., about 25 mg of carvedilol phosphate), the third unique interior volume includes the ARB pharmaceutical composition (e.g., about 25 mg losartan), the fourth unique interior volume includes aspirin (e.g., about 81 mg of aspirin), the fifth unique interior volume includes the first lipid-lowering pharmaceutical composition (e.g., about 40 mg of rosuvastatin), and the sixth unique interior volume includes the second lipid-lowering pharmaceutical composition (e.g., about 10 mg of ezetimibe).

In some embodiments, the first unique interior volume includes the CCB pharmaceutical composition (e.g., 120 mg of diltiazem), the second unique interior volume includes the BB pharmaceutical composition (e.g., about 25 mg of carvedilol phosphate), the third unique interior volume includes the ARB pharmaceutical composition (e.g., about 25 mg losartan), the fourth unique interior volume includes prasugrel (e.g., about 5 mg of prasugrel, about 10 mg of prasugrel), the fifth unique interior volume includes the first lipid-lowering pharmaceutical composition (e.g., about 40 mg of rosuvastatin), and the sixth unique interior volume includes the second lipid-lowering pharmaceutical composition (e.g., about 10 mg of ezetimibe).

In some embodiments, the first unique interior volume includes the CCB pharmaceutical composition (e.g., about 120 mg of diltiazem), the second unique interior volume includes the BB pharmaceutical composition (e.g., about 25 mg of carvedilol phosphate), the third unique interior volume includes the ARB pharmaceutical composition (e.g., about 25 mg losartan), the fourth unique interior volume includes the anti-platelet pharmaceutical composition (e.g., about 81 mg of aspirin, about 5 mg of prasugrel, about 10 mg of prasugrel), the fifth unique interior volume includes rosuvastatin (e.g., about 40 mg of rosuvastatin), and the sixth unique interior volume includes the second lipid-lowering pharmaceutical composition (e.g., about 10 mg of ezetimibe).

In some embodiments, the first unique interior volume includes the CCB pharmaceutical composition (e.g., about 120 mg of diltiazem), the second unique interior volume includes the BB pharmaceutical composition (e.g., about 25 mg of carvedilol phosphate), the third unique interior volume includes the ARB pharmaceutical composition (e.g., about 25 mg losartan), the fourth unique interior volume includes the anti-platelet pharmaceutical composition (e.g., about 81 mg of aspirin, about 5 mg of prasugrel, about 10 mg of prasugrel), the fifth unique interior volume includes the first lipid-lowering pharmaceutical composition (e.g., about 40 mg of rosuvastatin), and the sixth unique interior volume includes ezetimibe (e.g., about 10 mg of ezetimibe).

Furthermore, in some embodiments, the first unique interior volume includes the CCB pharmaceutical composition, the second unique interior volume includes the BB pharmaceutical composition, the third unique interior volume includes the ACEi pharmaceutical composition, the fourth unique interior volume includes the anti-platelet pharmaceutical composition, the fifth unique interior volume includes a first lipid-lowering pharmaceutical composition, the sixth unique interior volume includes a second lipid-lowering pharmaceutical composition, and the seventh unique interior volume includes the anti-anginal pharmaceutical composition. In some embodiments, a member selected from: the first unique interior volume, the third unique interior volume, the fourth unique interior volume, the fifth unique interior volume, the sixth unique interior volume, and the seventh unique interior; the first unique interior volume, the second unique interior volume, the fourth unique interior volume, the fifth unique interior volume, the sixth unique interior volume, and the seventh unique interior; the first unique interior volume, the second unique interior volume, the third unique interior volume, the fifth unique interior volume, the sixth unique interior volume, and the seventh unique interior; the first unique interior volume, the second unique interior volume, the third unique interior volume, the fourth unique interior volume, the sixth unique interior volume, and the seventh unique interior; the first unique interior volume, the second unique interior volume, the third unique interior volume, the fourth unique interior volume, the fifth unique interior volume, and the seventh unique interior; the first unique interior volume, the second unique interior volume, the third unique interior volume, the fourth unique interior volume, the fifth unique interior volume, and the sixth unique interior volume; the second unique interior volume, the third unique interior volume, the fourth unique interior volume, the fifth unique interior volume, the sixth unique interior volume, and the seventh unique interior do not include a class of pharmaceutical compositions that is the same as in another unique interior volume

In some embodiments, the first unique interior volume includes diltiazem (e.g., about 120 mg of diltiazem), the second unique interior volume includes the BB pharmaceutical composition (e.g., about 200 mg of metoprolol succinate), the third unique interior volume includes the ACEi pharmaceutical composition (e.g., about 5 mg of enalapril), the fourth unique interior volume includes the anti-platelet pharmaceutical composition (e.g., about 81 mg of aspirin, about 5 mg of prasugrel, about 10 mg of prasugrel), the fifth unique interior volume includes the first lipid-lowering pharmaceutical composition (e.g., about 20 mg of rosuvastatin), the sixth unique interior volume includes the second lipid-lowering pharmaceutical composition (e.g., about 10 mg of ezetimibe), and the seventh unique interior volume includes the anti-anginal pharmaceutical composition (e.g., about 30 mg of isosorbide mononitrate).

In some embodiments, the first unique interior volume includes the CCB pharmaceutical composition (e.g., about 120 mg of diltiazem), the second unique interior volume includes metoprolol succinate (e.g., about 200 mg of metoprolol succinate), the third unique interior volume includes the ACEi pharmaceutical composition (e.g., about 5 mg of enalapril), the fourth unique interior volume includes the anti-platelet pharmaceutical composition (e.g., about 81 mg of aspirin, about 5 mg of prasugrel, about 10 mg of prasugrel), the fifth unique interior volume includes the first lipid-lowering pharmaceutical composition (e.g., about 20 mg of rosuvastatin), the sixth unique interior volume includes the second lipid-lowering pharmaceutical composition (e.g., about 10 mg of ezetimibe), and the seventh unique interior volume includes the anti-anginal pharmaceutical composition (e.g., about 30 mg of isosorbide mononitrate).

In some embodiments, the first unique interior volume includes the CCB pharmaceutical composition (e.g., about 120 mg of diltiazem), the second unique interior volume includes the BB pharmaceutical composition (e.g., about 200 mg of metoprolol succinate), the third unique interior volume includes enalapril (e.g., about 5 mg of enalapril), the fourth unique interior volume includes the anti-platelet pharmaceutical composition (e.g., about 81 mg of aspirin, about 5 mg of prasugrel, about 10 mg of prasugrel), the fifth unique interior volume includes the first lipid-lowering pharmaceutical composition (e.g., about 20 mg of rosuvastatin), the sixth unique interior volume includes the second lipid-lowering pharmaceutical composition (e.g., about 10 mg of ezetimibe), and the seventh unique interior volume includes the anti-anginal pharmaceutical composition (e.g., about 30 mg of isosorbide mononitrate).

In some embodiments, the first unique interior volume includes the CCB pharmaceutical composition (e.g., about 120 mg of diltiazem), the second unique interior volume includes the BB pharmaceutical composition (e.g., about 200 mg of metoprolol succinate), the third unique interior volume includes the ACEi pharmaceutical composition (e.g., about 5 mg of enalapril), the fourth unique interior volume includes aspirin (e.g., about 81 mg of aspirin), the fifth unique interior volume includes the first lipid-lowering pharmaceutical composition (e.g., about 20 mg of rosuvastatin), the sixth unique interior volume includes the second lipid-lowering pharmaceutical composition (e.g., about 10 mg of ezetimibe), and the seventh unique interior volume includes the anti-anginal pharmaceutical composition (e.g., about 30 mg of isosorbide mononitrate).

In some embodiments, the first unique interior volume includes the CCB pharmaceutical composition (e.g., about 120 mg of diltiazem), the second unique interior volume includes the BB pharmaceutical composition (e.g., about 200 mg of metoprolol succinate), the third unique interior volume includes the ACEi pharmaceutical composition (e.g., about 5 mg of enalapril), the fourth unique interior volume includes prasugrel (e.g., about 81 mg of aspirin, about 5 mg of prasugrel, about 10 mg of prasugrel), the fifth unique interior volume includes the first lipid-lowering pharmaceutical composition (e.g., about 20 mg of rosuvastatin), the sixth unique interior volume includes the second lipid-lowering pharmaceutical composition (e.g., about 10 mg of ezetimibe), and the seventh unique interior volume includes the anti-anginal pharmaceutical composition (e.g., about 30 mg of isosorbide mononitrate).

In some embodiments, the first unique interior volume includes the CCB pharmaceutical composition (e.g., about 120 mg of diltiazem), the second unique interior volume includes the BB pharmaceutical composition (e.g., about 200 mg of metoprolol succinate), the third unique interior volume includes the ACEi pharmaceutical composition (e.g., about 5 mg of enalapril), the fourth unique interior volume includes the anti-platelet pharmaceutical composition (e.g., about 81 mg of aspirin, about 5 mg of prasugrel, about 10 mg of prasugrel), the fifth unique interior volume includes rosuvastatin (e.g., about 20 mg of rosuvastatin), the sixth unique interior volume includes the second lipid-lowering pharmaceutical composition (e.g., about 10 mg of ezetimibe), and the seventh unique interior volume includes the anti-anginal pharmaceutical composition (e.g., about 30 mg of isosorbide mononitrate).

In some embodiments, the first unique interior volume includes the CCB pharmaceutical composition (e.g., about 120 mg of diltiazem), the second unique interior volume includes the BB pharmaceutical composition (e.g., about 200 mg of metoprolol succinate), the third unique interior volume includes the ACEi pharmaceutical composition (e.g., about 5 mg of enalapril), the fourth unique interior volume includes the anti-platelet pharmaceutical composition (e.g., about 81 mg of aspirin, about 5 mg of prasugrel, about 10 mg of prasugrel), the fifth unique interior volume includes the first lipid-lowering pharmaceutical composition (e.g., about 20 mg of rosuvastatin), the sixth unique interior volume includes ezetimibe (e.g., about 10 mg of ezetimibe), and the seventh unique interior volume includes the anti-anginal pharmaceutical composition (e.g., about 30 mg of isosorbide mononitrate).

In some embodiments, the first unique interior volume includes the CCB pharmaceutical composition (e.g., about 120 mg of diltiazem), the second unique interior volume includes the BB pharmaceutical composition (e.g., about 200 mg of metoprolol succinate), the third unique interior volume includes the ACEi pharmaceutical composition (e.g., about 5 mg of enalapril), the fourth unique interior volume includes the anti-platelet pharmaceutical composition (e.g., about 81 mg of aspirin, about 5 mg of prasugrel, about 10 mg of prasugrel), the fifth unique interior volume includes the first lipid-lowering pharmaceutical composition (e.g., about 20 mg of rosuvastatin), the sixth unique interior volume includes the second lipid-lowering pharmaceutical composition (e.g., about 10 mg of ezetimibe), and the seventh unique interior volume includes isosorbide mononitrate (e.g., about 30 mg of isosorbide mononitrate).

Additionally, in some embodiments, the first unique interior volume includes the ACEi pharmaceutical composition, the second unique interior volume includes the BB pharmaceutical composition, the third unique interior volume includes the K-sparing diuretic pharmaceutical composition, and the fourth unique interior volume includes the K-wasting diuretic pharmaceutical composition. In some embodiments, a member selected from: the first unique interior volume, the second unique interior volume, and the third unique interior volume; the second unique interior volume, the third unique interior volume, the fourth unique interior volume; and the first unique interior volume, the third unique interior volume, and the fourth unique interior volume do not include a class of pharmaceutical compositions that is the same as in another unique interior volume.

In some embodiments, the first unique interior volume includes lisinopril (e.g., about 40 mg of lisinopril), the second unique interior volume includes the BB pharmaceutical composition (e.g., about 200 mg of metoprolol succinate), the third unique interior volume includes the K-sparing diuretic pharmaceutical composition (e.g., about 12.5 mg of spironolactone), and the fourth unique interior volume includes the K-wasting diuretic pharmaceutical composition (e.g., about 80 mg of furosemide).

In some embodiments, the first unique interior volume includes the ACEi pharmaceutical composition (e.g., about 40 mg of lisinopril), the second unique interior volume includes metoprolol succinate (e.g., about 200 mg of metoprolol succinate), the third unique interior volume includes the K-sparing diuretic pharmaceutical composition (e.g., about 12.5 mg of spironolactone), and the fourth unique interior volume includes the K-wasting diuretic pharmaceutical composition (e.g., about 80 mg of furosemide).

In some embodiments, the first unique interior volume includes the ACEi pharmaceutical composition (e.g., about 40 mg of lisinopril), the second unique interior volume includes the BB pharmaceutical composition (e.g., about 200 mg of metoprolol succinate), the third unique interior volume includes spironolactone (e.g., about 12.5 mg of spironolactone), and the fourth unique interior volume includes the K-wasting diuretic pharmaceutical composition (e.g., about 80 mg of furosemide).

In some embodiments, the first unique interior volume includes the ACEi pharmaceutical composition (e.g., about 40 mg of lisinopril), the second unique interior volume includes the BB pharmaceutical composition (e.g., about 200 mg of metoprolol succinate), the third unique interior volume includes the K-sparing diuretic pharmaceutical composition (e.g., about 12.5 mg of spironolactone), and the fourth unique interior volume includes furosemide (e.g., about 80 mg of furosemide).

Furthermore, in some embodiments, the first unique interior volume includes the BB pharmaceutical composition, the second unique interior volume includes the K-sparing diuretic pharmaceutical composition, the third unique interior volume includes the K-wasting diuretic pharmaceutical composition, and the fourth unique interior volume includes the anti-arrythmia pharmaceutical composition. In some embodiments, a member selected from: the first unique interior volume, the second unique interior volume, and the third unique interior volume; the second unique interior volume, the third unique interior volume, the fourth unique interior volume; and the first unique interior volume, the third unique interior volume, and the fourth unique interior volume do not include a class of pharmaceutical compositions that is the same as in another unique interior volume.

In some embodiments, the first unique interior volume includes metoprolol succinate (e.g., about 200 mg of metoprolol succinate), the second unique interior volume includes the K-sparing diuretic pharmaceutical composition (e.g., about 50 mg of eplerenone), the third unique interior volume includes the K-wasting diuretic pharmaceutical composition (e.g., about 40 mg of torsemide), and the fourth unique interior volume includes the anti-arrythmia pharmaceutical composition (e.g., about 1,200 mg of amiodarone).

In some embodiments, the first unique interior volume includes the BB pharmaceutical composition (e.g., about 200 mg of metoprolol succinate), the second unique interior volume includes eplerenone (e.g., about 50 mg of eplerenone), the third unique interior volume includes the K-wasting diuretic pharmaceutical composition (e.g., about 40 mg of torsemide), and the fourth unique interior volume includes the anti-arrythmia pharmaceutical composition (e.g., about 1,200 mg of amiodarone).

In some embodiments, the first unique interior volume includes the BB pharmaceutical composition (e.g., about 200 mg of metoprolol succinate), the second unique interior volume includes the K-sparing diuretic pharmaceutical composition (e.g., about 50 mg of eplerenone), the third unique interior volume includes torsemide (e.g., about 40 mg of torsemide), and the fourth unique interior volume includes the anti-arrythmia pharmaceutical composition (e.g., about 1,200 mg of amiodarone).

In some embodiments, the first unique interior volume includes the BB pharmaceutical composition (e.g., about 200 mg of metoprolol succinate), the second unique interior volume includes the K-sparing diuretic pharmaceutical composition (e.g., about 50 mg of eplerenone), the third unique interior volume includes the K-wasting diuretic pharmaceutical composition (e.g., about 40 mg of torsemide), and the fourth unique interior volume includes amiodarone (e.g., about 1,200 mg of amiodarone).

Moreover, in some embodiments, the first unique interior volume includes the ACEi pharmaceutical composition, the second unique interior volume includes the BB pharmaceutical composition, the third unique interior volume includes a first K-wasting diuretic pharmaceutical composition, and the fourth unique interior volume includes a second the K-wasting diuretic pharmaceutical composition. In some embodiments, a member selected from the first unique interior volume and the second unique interior volume, the second unique interior volume and the third unique interior volume, the first unique interior volume and the third unique interior volume, the second unique interior volume and the fourth unique interior volume, and the first unique interior volume and the fourth unique interior volume do not include a class of pharmaceutical compositions that is the same as in another unique interior volume.

In some embodiments, the first unique interior volume includes lisinopril (e.g., about 40 mg of lisinopril), the second unique interior volume includes the BB pharmaceutical composition (e.g., about 10 mg of bisoprolol), the third unique interior volume includes the first K-wasting diuretic pharmaceutical composition (e.g., about 4 mg of bumetanide, about 5 mg of metolazone), and the fourth unique interior volume includes the second K-wasting diuretic pharmaceutical composition (e.g., about 4 mg of bumetanide, about 5 mg of metolazone).

In some embodiments, the first unique interior volume includes the ACEi pharmaceutical composition (e.g., 4 about 0 mg of lisinopril), the second unique interior volume includes bisoprolol (e.g., about 10 mg of bisoprolol), the third unique interior volume includes the first K-wasting diuretic pharmaceutical composition (e.g., about 4 mg of bumetanide, about 5 mg of metolazone), and the fourth unique interior volume includes the second K-wasting diuretic pharmaceutical composition (e.g., about 4 mg of bumetanide, about 5 mg of metolazone).

In some embodiments, the first unique interior volume includes the ACEi pharmaceutical composition (e.g., about 40 mg of lisinopril), the second unique interior volume includes the BB pharmaceutical composition (e.g., about 10 mg of bisoprolol), the third unique interior volume includes bumetanide (e.g., about 4 mg of bumetanide), and the fourth unique interior volume includes the second K-wasting diuretic pharmaceutical composition (e.g., about 4 mg of bumetanide, about 5 mg of metolazone).

In some embodiments, the first unique interior volume includes the ACEi pharmaceutical composition (e.g., about 40 mg of lisinopril), the second unique interior volume includes the BB pharmaceutical composition (e.g., about 10 mg of bisoprolol), the third unique interior volume includes the first K-wasting diuretic pharmaceutical composition (e.g., about 4 mg of bumetanide, about 5 mg of metolazone), and the fourth unique interior volume includes metolazone (e.g., about 5 mg of metolazone).

Moreover, in some embodiments, the first unique interior volume includes the ACEi pharmaceutical composition, the second unique interior volume includes the BB pharmaceutical composition, the third unique interior volume includes the K-wasting diuretic pharmaceutical composition, and the fourth unique interior volume includes the anti-arrythmia pharmaceutical composition. In some embodiments, a member selected from the first unique interior volume and the second unique interior volume, the second unique interior volume and the third unique interior volume, the first unique interior volume and the third unique interior volume, the second unique interior volume and the fourth unique interior volume, and the first unique interior volume and the fourth unique interior volume do not include a class of pharmaceutical compositions that is the same as in another unique interior volume.

In some embodiments, the first unique interior volume includes ramipril (e.g., about 5 mg of ramipril), the second unique interior volume includes the BB pharmaceutical composition (e.g., about 100 mg of metoprolol succinate), the third unique interior volume includes the K-wasting diuretic pharmaceutical composition (e.g., about 4 mg of bumetanide), and the fourth unique interior volume includes the anti-arrythmia pharmaceutical composition (e.g., about 1,200 mg of amiodarone).

In some embodiments, the first unique interior volume includes the ACEi pharmaceutical composition (e.g., about 5 mg of ramipril), the second unique interior volume includes metoprolol succinate (e.g., about 100 mg of metoprolol succinate), the third unique interior volume includes the K-wasting diuretic pharmaceutical composition (e.g., about 4 mg of bumetanide), and the fourth unique interior volume includes the anti-arrythmia pharmaceutical composition (e.g., about 1,200 mg of amiodarone).

In some embodiments, the first unique interior volume includes the ACEi pharmaceutical composition (e.g., about 5 mg of ramipril), the second unique interior volume includes the BB pharmaceutical composition (e.g., about 100 mg of metoprolol succinate), the third unique interior volume includes bumetanide (e.g., about 4 mg of bumetanide), and the fourth unique interior volume includes the anti-arrythmia pharmaceutical composition (e.g., about 400 mg of amiodarone).

In some embodiments, the first unique interior volume includes the ACEi pharmaceutical composition (e.g., about 5 mg of ramipril), the second unique interior volume includes the BB pharmaceutical composition (e.g., about 100 mg of metoprolol succinate), the third unique interior volume includes the K-wasting diuretic pharmaceutical composition (e.g., about 4 mg of bumetanide), and the fourth unique interior volume includes amiodarone (e.g., about 1,200 mg of amiodarone).

Furthermore, in some embodiments, the first unique interior volume includes the ARB pharmaceutical composition, the second unique interior volume includes the BB pharmaceutical composition, the third unique interior volume includes the K-sparing diuretic pharmaceutical composition, the fourth unique interior volume includes the first K-wasting diuretic pharmaceutical composition, the fifth unique interior volume includes the second K-wasting diuretic pharmaceutical composition, and the sixth unique interior volume includes the anti-arrythmia pharmaceutical composition. In some embodiments, a member selected from: the first unique interior volume, the second unique interior volume, the third unique interior volume, the fourth interior volume, and the fifth unique interior volume; the first unique interior volume, the second unique interior volume, the third unique interior volume, the fourth unique interior volume, and the sixth unique interior volume; the first unique interior volume, the second unique interior volume, the third unique interior volume, the fifth unique interior volume, and the sixth unique interior volume; the first unique interior volume, the second unique interior volume, the fourth unique interior volume, the fifth unique interior volume, and the sixth unique interior volume; the first unique interior volume, the third unique interior volume, the fourth unique interior volume, the fifth unique interior volume, and the sixth unique interior volume; the second unique interior volume, the third unique interior volume, the fourth unique interior volume, the fifth unique interior volume, and the sixth unique interior volume do not include a class of pharmaceutical compositions that is the same as in another unique interior volume.

In some embodiments, the first unique interior volume includes valsartan (e.g., about 80 mg of valsartan), the second unique interior volume includes the BB pharmaceutical composition (e.g., about 25 mg of carvedilol phosphate), the third unique interior volume includes the K-sparing pharmaceutical composition (e.g., about 12.5 mg of spironolactone), the fourth unique interior volume includes the first K-wasting diuretic pharmaceutical composition (e.g., about 80 mg of torsemide), the fifth unique interior volume includes the second K-wasting diuretic pharmaceutical composition (e.g., about 2.5 mg of metolazone), and the sixth unique interior volume includes the anti-arrythmia pharmaceutical composition (e.g., about 1,200 mg of amiodarone).

In some embodiments, the first unique interior volume includes the ARB pharmaceutical composition (e.g., about 80 mg of valsartan), the second unique interior volume includes carvedilol phosphate (e.g., about 25 mg of carvedilol phosphate), the third unique interior volume includes the K-sparing pharmaceutical composition (e.g., about 12.5 mg of spironolactone), the fourth unique interior volume includes the first K-wasting diuretic pharmaceutical composition (e.g., about 80 mg of torsemide), the fifth unique interior volume includes the second K-wasting diuretic pharmaceutical composition (e.g., about 2.5 mg of metolazone), and the sixth unique interior volume includes the anti-arrythmia pharmaceutical composition (e.g., about 1,200 mg of amiodarone).

In some embodiments, the first unique interior volume includes the ARB pharmaceutical composition (e.g., about 80 mg of valsartan), the second unique interior volume includes the BB pharmaceutical composition (e.g., about 25 mg of carvedilol phosphate), the third unique interior volume includes spironolactone (e.g., about 12.5 mg of spironolactone), the fourth unique interior volume includes the first K-wasting diuretic pharmaceutical composition (e.g., about 80 mg of torsemide), the fifth unique interior volume includes the second K-wasting diuretic pharmaceutical composition (e.g., about 2.5 mg of metolazone), and the sixth unique interior volume includes the anti-arrythmia pharmaceutical composition (e.g., about 200 mg of amiodarone).

In some embodiments, the first unique interior volume includes the ARB pharmaceutical composition (e.g., about 80 mg of valsartan), the second unique interior volume includes the BB pharmaceutical composition (e.g., about 25 mg of carvedilol phosphate), the third unique interior volume includes the K-sparing pharmaceutical composition (e.g., about 12.5 mg of spironolactone), the fourth unique interior volume includes torsemide (e.g., about 80 mg of torsemide), the fifth unique interior volume includes the second K-wasting diuretic pharmaceutical composition (e.g., about 2.5 mg of metolazone), and the sixth unique interior volume includes the anti-arrythmia pharmaceutical composition (e.g., about 1,600 mg of amiodarone).

In some embodiments, the first unique interior volume includes the ARB pharmaceutical composition (e.g., about 80 mg of valsartan), the second unique interior volume includes the BB pharmaceutical composition (e.g., about 25 mg of carvedilol phosphate), the third unique interior volume includes the K-sparing pharmaceutical composition (e.g., about 12.5 mg of spironolactone), the fourth unique interior volume includes the first K-wasting diuretic pharmaceutical composition (e.g., about 80 mg of torsemide), the fifth unique interior volume includes metolazone (e.g., about 2.5 mg of metolazone), and the sixth unique interior volume includes the anti-arrythmia pharmaceutical composition (e.g., about 400 mg of amiodarone).

In some embodiments, the first unique interior volume includes the ARB pharmaceutical composition (e.g., about 80 mg of valsartan), the second unique interior volume includes the BB pharmaceutical composition (e.g., about 25 mg of carvedilol phosphate), the third unique interior volume includes the K-sparing pharmaceutical composition (e.g., about 12.5 mg of spironolactone), the fourth unique interior volume includes the first K-wasting diuretic pharmaceutical composition (e.g., about 80 mg of torsemide), the fifth unique interior volume includes the second K-wasting diuretic pharmaceutical composition (e.g., about 2.5 mg of metolazone), and the sixth unique interior volume includes amiodarone (e.g., about 1,200 mg of amiodarone).

Furthermore, in some embodiments, the first unique interior volume includes the anti-platelet pharmaceutical composition, the second unique interior volume includes a first lipid-lowering pharmaceutical composition, the third unique interior volume includes a second lipid-lowering pharmaceutical composition, the fourth unique interior volume includes the BB pharmaceutical composition, the fifth unique interior volume includes the ACEi pharmaceutical composition, and the sixth unique interior volume includes the thiazide pharmaceutical composition. In some embodiments, a member selected from: the first unique interior volume, the second unique interior volume, the third unique interior volume, the fourth interior volume, and the fifth unique interior volume; the first unique interior volume, the second unique interior volume, the third unique interior volume, the fourth unique interior volume, and the sixth unique interior volume; the first unique interior volume, the second unique interior volume, the third unique interior volume, the fifth unique interior volume, and the sixth unique interior volume; the first unique interior volume, the second unique interior volume, the fourth unique interior volume, the fifth unique interior volume, and the sixth unique interior volume; the first unique interior volume, the third unique interior volume, the fourth unique interior volume, the fifth unique interior volume, and the sixth unique interior volume; the second unique interior volume, the third unique interior volume, the fourth unique interior volume, the fifth unique interior volume, and the sixth unique interior volume do not include a class of pharmaceutical compositions that is the same as in another unique interior volume.

In some embodiments, the first unique interior volume includes aspirin (e.g., about 81 mg of aspirin), the second unique interior volume includes the first lipid-lowering pharmaceutical composition (e.g., about 80 mg of atorvastatin), the third unique interior volume includes the second lipid-lowering composition (e.g., about 10 mg of ezetimibe), the fourth unique interior volume includes the BB pharmaceutical composition (e.g., about 150 mg of metoprolol succinate), the fifth unique interior volume includes the ACEi pharmaceutical composition (e.g., about 5 mg of lisinopril), and the sixth unique interior volume includes the thiazide pharmaceutical composition (e.g., about 12.5 mg of hydrochlorothiazide).

In some embodiments, the first unique interior volume includes prasugrel (e.g., about 5 mg of prasugrel, about 10 mg of prasugrel), the second unique interior volume includes the first lipid-lowering pharmaceutical composition (e.g., about 80 mg of atorvastatin), the third unique interior volume includes the second lipid-lowering composition (e.g., about 10 mg of ezetimibe), the fourth unique interior volume includes the BB pharmaceutical composition (e.g., about 150 mg of metoprolol succinate), the fifth unique interior volume includes the ACEi pharmaceutical composition (e.g., about 5 mg of lisinopril), and the sixth unique interior volume includes the thiazide pharmaceutical composition (e.g., about 12.5 mg of hydrochlorothiazide).

In some embodiments, the first unique interior volume includes the anti-platelet pharmaceutical composition (e.g., about 81 mg of aspirin, about 5 mg of prasugrel, about 10 mg of prasugrel), the second unique interior volume includes atorvastatin (e.g., about 80 mg of atorvastatin), the third unique interior volume includes the second lipid-lowering composition (e.g., about 10 mg of ezetimibe), the fourth unique interior volume includes the BB pharmaceutical composition (e.g., about 150 mg of metoprolol succinate), the fifth unique interior volume includes the ACEi pharmaceutical composition (e.g., about 5 mg of lisinopril), and the sixth unique interior volume includes the thiazide pharmaceutical composition (e.g., about 12.5 mg of hydrochlorothiazide).

In some embodiments, the first unique interior volume includes the anti-platelet pharmaceutical composition (e.g., about 81 mg of aspirin, about 5 mg of prasugrel, about 10 mg of prasugrel), the second unique interior volume includes the first lipid-lowering pharmaceutical composition (e.g., about 80 mg of atorvastatin), the third unique interior volume includes ezetimibe (e.g., about 10 mg of ezetimibe), the fourth unique interior volume includes the BB pharmaceutical composition (e.g., about 150 mg of metoprolol succinate), the fifth unique interior volume includes the ACEi pharmaceutical composition (e.g., about 5 mg of lisinopril), and the sixth unique interior volume includes the thiazide pharmaceutical composition (e.g., about 12.5 mg of hydrochlorothiazide).

In some embodiments, the first unique interior volume includes the anti-platelet pharmaceutical composition (e.g., about 81 mg of aspirin, about 5 mg of prasugrel, about 10 mg of prasugrel), the second unique interior volume includes the first lipid-lowering pharmaceutical composition (e.g., about 80 mg of atorvastatin), the third unique interior volume includes the second lipid-lowering composition (e.g., about 10 mg of ezetimibe), the fourth unique interior volume includes metoprolol succinate (e.g., about 150 mg of metoprolol succinate), the fifth unique interior volume includes the ACEi pharmaceutical composition (e.g., about 5 mg of lisinopril), and the sixth unique interior volume includes the thiazide pharmaceutical composition (e.g., about 12.5 mg of hydrochlorothiazide).

In some embodiments, the first unique interior volume includes the anti-platelet pharmaceutical composition (e.g., about 81 mg of aspirin, about 5 mg of prasugrel, about 10 mg of prasugrel), the second unique interior volume includes the first lipid-lowering pharmaceutical composition (e.g., about 80 mg of atorvastatin), the third unique interior volume includes the second lipid-lowering composition (e.g., about 10 mg of ezetimibe), the fourth unique interior volume includes the BB pharmaceutical composition (e.g., about 150 mg of metoprolol succinate), the fifth unique interior volume includes lisinopril (e.g., about 5 mg of lisinopril), and the sixth unique interior volume includes the thiazide pharmaceutical composition (e.g., about 12.5 mg of hydrochlorothiazide).

In some embodiments, the first unique interior volume includes the anti-platelet pharmaceutical composition (e.g., about 81 mg of aspirin, about 5 mg of prasugrel, about 10 mg of prasugrel), the second unique interior volume includes the first lipid-lowering pharmaceutical composition (e.g., about 80 mg of atorvastatin), the third unique interior volume includes the second lipid-lowering composition (e.g., about 10 mg of ezetimibe), the fourth unique interior volume includes the BB pharmaceutical composition (e.g., about 150 mg of metoprolol succinate), the fifth unique interior volume includes the ACEi pharmaceutical composition (e.g., about 5 mg of lisinopril), and the sixth unique interior volume includes hydrochlorothiazide (e.g., about 12.5 mg of hydrochlorothiazide).

Furthermore, in some embodiments, the first unique interior volume includes the anti-platelet pharmaceutical composition, the second unique interior volume includes the first lipid-lowering pharmaceutical composition, the third unique interior volume includes the second lipid-lowering pharmaceutical composition, the fourth unique interior volume includes the BB pharmaceutical composition, the fifth unique interior volume includes the ACEi pharmaceutical composition, the sixth unique interior volume includes the K-sparing diuretic pharmaceutical composition, and the seventh unique interior volume includes the K-wasting diuretic pharmaceutical composition. In some embodiments, a member selected from: the first unique interior volume, the third unique interior volume, the fourth unique interior volume, the fifth unique interior volume, the sixth unique interior volume, and the seventh unique interior; the first unique interior volume, the second unique interior volume, the fourth unique interior volume, the fifth unique interior volume, the sixth unique interior volume, and the seventh unique interior; the first unique interior volume, the second unique interior volume, the third unique interior volume, the fifth unique interior volume, the sixth unique interior volume, and the seventh unique interior; the first unique interior volume, the second unique interior volume, the third unique interior volume, the fourth unique interior volume, the sixth unique interior volume, and the seventh unique interior; the first unique interior volume, the second unique interior volume, the third unique interior volume, the fourth unique interior volume, the fifth unique interior volume, and the seventh unique interior; the first unique interior volume, the second unique interior volume, the third unique interior volume, the fourth unique interior volume, the fifth unique interior volume, and the sixth unique interior volume; the second unique interior volume, the third unique interior volume, the fourth unique interior volume, the fifth unique interior volume, the sixth unique interior volume, and the seventh unique interior do not include a class of pharmaceutical compositions that is the same as in another unique interior volume.

In some embodiments, the first unique interior volume includes aspirin (e.g., about 81 mg of aspirin), the second unique interior volume includes the first lipid-lowering pharmaceutical composition (e.g., about 40 mg of atorvastatin), the third unique interior volume includes the second lipid-lowering composition (e.g., about 10 mg of ezetimibe), the fourth unique interior volume includes the BB pharmaceutical composition (e.g., about 200 mg of metoprolol succinate), the fifth unique interior volume includes the ACEi pharmaceutical composition (e.g., about 20 mg of enalapril), the sixth unique interior volume includes the K-sparing diuretic pharmaceutical composition (e.g., about 12.5 mg spironolactone), and the seventh unique interior volume includes the K-wasting diuretic pharmaceutical composition (e.g., about 120 mg of torsemide).

In some embodiments, the first unique interior volume includes prasugrel (e.g., about 5 mg of prasugrel, about 10 mg of prasugrel), the second unique interior volume includes the first lipid-lowering pharmaceutical composition (e.g., about 40 mg of atorvastatin), the third unique interior volume includes the second lipid-lowering composition (e.g., about 10 mg of ezetimibe), the fourth unique interior volume includes the BB pharmaceutical composition (e.g., about 200 mg of metoprolol succinate), the fifth unique interior volume includes the ACEi pharmaceutical composition (e.g., about 20 mg of enalapril), the sixth unique interior volume includes the K-sparing diuretic pharmaceutical composition (e.g., about 12.5 mg spironolactone), and the seventh unique interior volume includes the K-wasting diuretic pharmaceutical composition (e.g., about 120 mg of torsemide).

In some embodiments, the first unique interior volume includes the anti-platelet pharmaceutical composition (e.g., about 81 mg of aspirin, about 5 mg of prasugrel, about 10 mg of prasugrel), the second unique interior volume includes atorvastatin (e.g., about 40 mg of atorvastatin), the third unique interior volume includes the second lipid-lowering composition (e.g., about 10 mg of ezetimibe), the fourth unique interior volume includes the BB pharmaceutical composition (e.g., about 200 mg of metoprolol succinate), the fifth unique interior volume includes the ACEi pharmaceutical composition (e.g., about 20 mg of enalapril), the sixth unique interior volume includes the K-sparing diuretic pharmaceutical composition (e.g., about 12.5 mg spironolactone), and the seventh unique interior volume includes the K-wasting diuretic pharmaceutical composition (e.g., about 120 mg of torsemide).

In some embodiments, the first unique interior volume includes the anti-platelet pharmaceutical composition (e.g., about 81 mg of aspirin, about 5 mg of prasugrel, about 10 mg of prasugrel), the second unique interior volume includes the first lipid-lowering pharmaceutical composition (e.g., about 40 mg of atorvastatin), the third unique interior volume includes ezetimibe (e.g., about 10 mg of ezetimibe), the fourth unique interior volume includes the BB pharmaceutical composition (e.g., about 200 mg of metoprolol succinate), the fifth unique interior volume includes the ACEi pharmaceutical composition (e.g., about 20 mg of enalapril), the sixth unique interior volume includes the K-sparing diuretic pharmaceutical composition (e.g., about 12.5 mg spironolactone), and the seventh unique interior volume includes the K-wasting diuretic pharmaceutical composition (e.g., about 120 mg of torsemide).

In some embodiments, the first unique interior volume includes the anti-platelet pharmaceutical composition (e.g., about 81 mg of aspirin, about 5 mg of prasugrel, about 10 mg of prasugrel), the second unique interior volume includes the first lipid-lowering pharmaceutical composition (e.g., about 40 mg of atorvastatin), the third unique interior volume includes the second lipid-lowering composition (e.g., about 10 mg of ezetimibe), the fourth unique interior volume includes metoprolol succinate (e.g., about 200 mg of metoprolol succinate), the fifth unique interior volume includes the ACEi pharmaceutical composition (e.g., about 20 mg of enalapril), the sixth unique interior volume includes the K-sparing diuretic pharmaceutical composition (e.g., about 12.5 mg spironolactone), and the seventh unique interior volume includes the K-wasting diuretic pharmaceutical composition (e.g., about 120 mg of torsemide).

In some embodiments, the first unique interior volume includes the anti-platelet pharmaceutical composition (e.g., about 81 mg of aspirin, about 5 mg of prasugrel, about 10 mg of prasugrel), the second unique interior volume includes the first lipid-lowering pharmaceutical composition (e.g., about 40 mg of atorvastatin), the third unique interior volume includes the second lipid-lowering composition (e.g., about 10 mg of ezetimibe), the fourth unique interior volume includes the BB pharmaceutical composition (e.g., about 200 mg of metoprolol succinate), the fifth unique interior volume includes enalapril (e.g., about 20 mg of enalapril), the sixth unique interior volume includes the K-sparing diuretic pharmaceutical composition (e.g., about 12.5 mg spironolactone), and the seventh unique interior volume includes the K-wasting diuretic pharmaceutical composition (e.g., about 120 mg of torsemide).

In some embodiments, the first unique interior volume includes the anti-platelet pharmaceutical composition (e.g., about 81 mg of aspirin, about 5 mg of prasugrel, about 10 mg of prasugrel), the second unique interior volume includes the first lipid-lowering pharmaceutical composition (e.g., about 40 mg of atorvastatin), the third unique interior volume includes the second lipid-lowering composition (e.g., about 10 mg of ezetimibe), the fourth unique interior volume includes the BB pharmaceutical composition (e.g., about 200 mg of metoprolol succinate), the fifth unique interior volume includes the ACEi pharmaceutical composition (e.g., about 20 mg of enalapril), the sixth unique interior volume includes spironolactone (e.g., about 12.5 mg spironolactone), and the seventh unique interior volume includes the K-wasting diuretic pharmaceutical composition (e.g., about 120 mg of torsemide).

In some embodiments, the first unique interior volume includes the anti-platelet pharmaceutical composition (e.g., about 81 mg of aspirin, about 5 mg of prasugrel, about 10 mg of prasugrel), the second unique interior volume includes the first lipid-lowering pharmaceutical composition (e.g., about 40 mg of atorvastatin), the third unique interior volume includes the second lipid-lowering composition (e.g., about 10 mg of ezetimibe), the fourth unique interior volume includes the BB pharmaceutical composition (e.g., about 200 mg of metoprolol succinate), the fifth unique interior volume includes the ACEi pharmaceutical composition (e.g., about 20 mg of enalapril), the sixth unique interior volume includes the K-sparing diuretic pharmaceutical composition (e.g., about 12.5 mg spironolactone), and the seventh unique interior volume includes torsemide (e.g., about 120 mg of torsemide).

Furthermore, in some embodiments, the first unique interior volume includes the anti-platelet pharmaceutical composition, the second unique interior volume includes the first lipid-lowering pharmaceutical composition, the third unique interior volume includes the second lipid-lowering pharmaceutical composition, the fourth unique interior volume includes the BB pharmaceutical composition, the fifth unique interior volume includes the ARB pharmaceutical composition, the sixth unique interior volume includes the K-sparing diuretic pharmaceutical composition, the seventh unique interior volume includes the K-wasting diuretic pharmaceutical composition, and the eighth unique interior volume includes the CCB pharmaceutical composition. In some embodiments, a member selected from: the first unique interior volume, the second unique interior volume, the third unique interior volume, the fourth unique interior volume, the fifth unique interior volume, the sixth unique interior volume, and the seventh unique interior; the first unique interior volume, the second unique interior volume, the third unique interior volume, the fourth unique interior volume, the fifth unique interior volume, the sixth unique interior volume, and the eighth unique interior volume; the first unique interior volume, the second unique interior volume, the third unique interior volume, the fourth unique interior volume, the fifth unique interior volume, the seventh unique interior, and the eighth unique interior volume; the first unique interior volume, the second unique interior volume, the third unique interior volume, the fourth unique interior volume, the sixth unique interior volume, the seventh unique interior, and the eighth unique interior volume; the first unique interior volume, the second unique interior volume, the third unique interior volume, the fifth unique interior volume, the sixth unique interior volume, the seventh unique interior, and the eighth unique interior volume; the first unique interior volume, the second unique interior volume, the fourth unique interior volume, the fifth unique interior volume, the sixth unique interior volume, the seventh unique interior, and the eighth unique interior volume; the first unique interior volume, the third unique interior volume, the fourth unique interior volume, the fifth unique interior volume, the sixth unique interior volume, the seventh unique interior, and the eighth unique interior volume; the second unique interior volume, the third unique interior volume, the fourth unique interior volume, the fifth unique interior volume, the sixth unique interior volume, the seventh unique interior, and the eighth unique interior volume do not include a class of pharmaceutical compositions that is the same as in another unique interior volume.

In some embodiments, the first unique interior volume includes aspirin (e.g., about 81 mg of aspirin), the second unique interior volume includes the first lipid-lowering pharmaceutical composition (e.g., about 80 mg of atorvastatin), the third unique interior volume includes the second lipid-lowering composition (e.g., about 10 mg of ezetimibe), the fourth unique interior volume includes the BB pharmaceutical composition (e.g., about 200 mg of metoprolol succinate), the fifth unique interior volume includes the ARB pharmaceutical composition (e.g., about 80 mg of valsartan), the sixth unique interior volume includes the K-sparing diuretic pharmaceutical composition (e.g., about 12.5 mg spironolactone), the seventh unique interior volume includes the K-wasting diuretic pharmaceutical composition (e.g., about 160 mg of furosemide), and the eighth unique interior volume includes the CCB pharmaceutical composition (e.g., about 5 mg of amlodipine).

In some embodiments, the first unique interior volume includes prasugrel (e.g., about 5 mg of prasugrel, about 10 mg of prasugrel), the second unique interior volume includes the first lipid-lowering pharmaceutical composition (e.g., about 80 mg of atorvastatin), the third unique interior volume includes the second lipid-lowering composition (e.g., about 10 mg of ezetimibe), the fourth unique interior volume includes the BB pharmaceutical composition (e.g., about 200 mg of metoprolol succinate), the fifth unique interior volume includes the ARB pharmaceutical composition (e.g., about 80 mg of valsartan), the sixth unique interior volume includes the K-sparing diuretic pharmaceutical composition (e.g., about 12.5 mg spironolactone), the seventh unique interior volume includes the K-wasting diuretic pharmaceutical composition (e.g., about 160 mg of furosemide), and the eighth unique interior volume includes the CCB pharmaceutical composition (e.g., about 5 mg of amlodipine).

In some embodiments, the first unique interior volume includes the anti-platelet pharmaceutical composition (e.g., about 81 mg of aspirin, about 5 mg of prasugrel, about 10 mg of prasugrel), the second unique interior volume includes atorvastatin (e.g., about 80 mg of atorvastatin), the third unique interior volume includes the second lipid-lowering composition (e.g., about 10 mg of ezetimibe), the fourth unique interior volume includes the BB pharmaceutical composition (e.g., about 200 mg of metoprolol succinate), the fifth unique interior volume includes the ARB pharmaceutical composition (e.g., about 80 mg of valsartan), the sixth unique interior volume includes the K-sparing diuretic pharmaceutical composition (e.g., about 12.5 mg spironolactone), the seventh unique interior volume includes the K-wasting diuretic pharmaceutical composition (e.g., about 160 mg of furosemide), and the eighth unique interior volume includes the CCB pharmaceutical composition (e.g., about 5 mg of amlodipine).

In some embodiments, the first unique interior volume includes the anti-platelet pharmaceutical composition (e.g., about 81 mg of aspirin, about 5 mg of prasugrel, about 10 mg of prasugrel), the second unique interior volume includes the first lipid-lowering pharmaceutical composition (e.g., about 80 mg of atorvastatin), the third unique interior volume includes ezetimibe (e.g., about 10 mg of ezetimibe), the fourth unique interior volume includes the BB pharmaceutical composition (e.g., about 200 mg of metoprolol succinate), the fifth unique interior volume includes the ARB pharmaceutical composition (e.g., about 80 mg of valsartan), the sixth unique interior volume includes the K-sparing diuretic pharmaceutical composition (e.g., about 12.5 mg spironolactone), the seventh unique interior volume includes the K-wasting diuretic pharmaceutical composition (e.g., about 160 mg of furosemide), and the eighth unique interior volume includes the CCB pharmaceutical composition (e.g., about 5 mg of amlodipine).

In some embodiments, the first unique interior volume includes the anti-platelet pharmaceutical composition (e.g., about 81 mg of aspirin, about 5 mg of prasugrel, about 10 mg of prasugrel), the second unique interior volume includes the first lipid-lowering pharmaceutical composition (e.g., about 80 mg of atorvastatin), the third unique interior volume includes the second lipid-lowering composition (e.g., about 10 mg of ezetimibe), the fourth unique interior volume includes metoprolol succinate (e.g., about 200 mg of metoprolol succinate), the fifth unique interior volume includes the ARB pharmaceutical composition (e.g., about 80 mg of valsartan), the sixth unique interior volume includes the K-sparing diuretic pharmaceutical composition (e.g., about 12.5 mg spironolactone), the seventh unique interior volume includes the K-wasting diuretic pharmaceutical composition (e.g., about 160 mg of furosemide), and the eighth unique interior volume includes the CCB pharmaceutical composition (e.g., about 5 mg of amlodipine).

In some embodiments, the first unique interior volume includes the anti-platelet pharmaceutical composition (e.g., about 81 mg of aspirin, about 5 mg of prasugrel, about 10 mg of prasugrel), the second unique interior volume includes the first lipid-lowering pharmaceutical composition (e.g., about 80 mg of atorvastatin), the third unique interior volume includes the second lipid-lowering composition (e.g., about 10 mg of ezetimibe), the fourth unique interior volume includes the BB pharmaceutical composition (e.g., about 200 mg of metoprolol succinate), the fifth unique interior volume includes valsartan (e.g., about 80 mg of valsartan), the sixth unique interior volume includes the K-sparing diuretic pharmaceutical composition (e.g., about 12.5 mg spironolactone), the seventh unique interior volume includes the K-wasting diuretic pharmaceutical composition (e.g., about 160 mg of furosemide), and the eighth unique interior volume includes the CCB pharmaceutical composition (e.g., about 5 mg of amlodipine).

In some embodiments, the first unique interior volume includes the anti-platelet pharmaceutical composition (e.g., about 81 mg of aspirin, about 5 mg of prasugrel, about 10 mg of prasugrel), the second unique interior volume includes the first lipid-lowering pharmaceutical composition (e.g., about 80 mg of atorvastatin), the third unique interior volume includes the second lipid-lowering composition (e.g., about 10 mg of ezetimibe), the fourth unique interior volume includes the BB pharmaceutical composition (e.g., about 200 mg of metoprolol succinate), the fifth unique interior volume includes the ARB pharmaceutical composition (e.g., about 80 mg of valsartan), the sixth unique interior volume includes spironolactone (e.g., about 12.5 mg spironolactone), the seventh unique interior volume includes the K-wasting diuretic pharmaceutical composition (e.g., about 160 mg of furosemide), and the eighth unique interior volume includes the CCB pharmaceutical composition (e.g., about 5 mg of amlodipine).

In some embodiments, the first unique interior volume includes the anti-platelet pharmaceutical composition (e.g., about 81 mg of aspirin, about 5 mg of prasugrel, about 10 mg of prasugrel), the second unique interior volume includes the first lipid-lowering pharmaceutical composition (e.g., about 80 mg of atorvastatin), the third unique interior volume includes the second lipid-lowering composition (e.g., about 10 mg of ezetimibe), the fourth unique interior volume includes the BB pharmaceutical composition (e.g., about 200 mg of metoprolol succinate), the fifth unique interior volume includes the ARB pharmaceutical composition (e.g., about 80 mg of valsartan), the sixth unique interior volume includes the K-sparing diuretic pharmaceutical composition (e.g., about 12.5 mg spironolactone), the seventh unique interior volume includes furosemide (e.g., about 160 mg of furosemide), and the eighth unique interior volume includes the CCB pharmaceutical composition (e.g., about 5 mg of amlodipine).

In some embodiments, the first unique interior volume includes the anti-platelet pharmaceutical composition (e.g., about 81 mg of aspirin, about 5 mg of prasugrel, about 10 mg of prasugrel), the second unique interior volume includes the first lipid-lowering pharmaceutical composition (e.g., about 80 mg of atorvastatin), the third unique interior volume includes the second lipid-lowering composition (e.g., about 10 mg of ezetimibe), the fourth unique interior volume includes the BB pharmaceutical composition (e.g., about 200 mg of metoprolol succinate), the fifth unique interior volume includes the ARB pharmaceutical composition (e.g., about 80 mg of valsartan), the sixth unique interior volume includes the K-sparing diuretic pharmaceutical composition (e.g., about 12.5 mg spironolactone), the seventh unique interior volume includes the K-wasting diuretic pharmaceutical composition (e.g., about 160 mg of furosemide), and the eighth unique interior volume includes amlodipine (e.g., about 5 mg of amlodipine).

Additionally, in some embodiments, the first unique interior volume includes the ACEi pharmaceutical composition, the second unique interior volume includes the BB pharmaceutical composition, the third unique interior volume includes the CCB pharmaceutical composition, the fourth unique interior volume includes the K-sparing diuretic pharmaceutical composition, and the fifth unique interior volume includes the K-wasting diuretic pharmaceutical composition. In some embodiments, a member selected from: the first unique interior volume, the second unique interior volume, the third unique interior volume, and the fourth interior volume; the first unique interior volume, the second unique interior volume, the third unique interior volume, and the fifth unique interior volume; the first unique interior volume, the second unique interior volume, the fourth unique interior volume, and the fifth unique interior volume; the second unique interior volume, the third unique interior volume, the fourth unique interior volume, and the fifth unique interior do not include a class of pharmaceutical compositions that is the same as in another unique interior volume.

In some embodiments, the first unique interior volume includes lisinopril (e.g., about 40 mg of lisinopril), the second unique interior volume includes the BB pharmaceutical composition (e.g., about 50 mg of carvedilol phosphate), the third unique interior volume includes the CCB pharmaceutical composition (e.g., about 5 mg of amlodipine), the fourth unique interior volume includes the K-sparing diuretic pharmaceutical composition (e.g., about 25 mg of spironolactone), and the fifth unique interior volume includes the K-wasting diuretic pharmaceutical composition (e.g., about 200 mg of furosemide).

In some embodiments, the first unique interior volume includes the ACEi pharmaceutical composition (e.g., about 40 mg of lisinopril), the second unique interior volume includes carvedilol phosphate (e.g., about 50 mg of carvedilol phosphate), the third unique interior volume includes the CCB pharmaceutical composition (e.g., about 5 mg of amlodipine), the fourth unique interior volume includes the K-sparing diuretic pharmaceutical composition (e.g., about 25 mg of spironolactone), and the fifth unique interior volume includes the K-wasting diuretic pharmaceutical composition (e.g., about 200 mg of furosemide).

In some embodiments, the first unique interior volume includes the ACEi pharmaceutical composition (e.g., about 40 mg of lisinopril), the second unique interior volume includes the BB pharmaceutical composition (e.g., about 50 mg of carvedilol phosphate), the third unique interior volume includes amlodipine (e.g., about 5 mg of amlodipine), the fourth unique interior volume includes the K-sparing diuretic pharmaceutical composition (e.g., about 25 mg of spironolactone), and the fifth unique interior volume includes the K-wasting diuretic pharmaceutical composition (e.g., about 200 mg of furosemide).

In some embodiments, the first unique interior volume includes the ACEi pharmaceutical composition (e.g., about 40 mg of lisinopril), the second unique interior volume includes the BB pharmaceutical composition (e.g., about 50 mg of carvedilol phosphate), the third unique interior volume includes the CCB pharmaceutical composition (e.g., about 5 mg of amlodipine), the fourth unique interior volume includes spironolactone (e.g., about 25 mg of spironolactone), and the fifth unique interior volume includes the K-wasting diuretic pharmaceutical composition (e.g., about 200 mg of furosemide).

In some embodiments, the first unique interior volume includes the ACEi pharmaceutical composition (e.g., about 40 mg of lisinopril), the second unique interior volume includes the BB pharmaceutical composition (e.g., about 50 mg of carvedilol phosphate), the third unique interior volume includes the CCB pharmaceutical composition (e.g., about 5 mg of amlodipine), the fourth unique interior volume includes the K-sparing diuretic pharmaceutical composition (e.g., about 25 mg of spironolactone), and the fifth unique interior volume includes furosemide (e.g., about 200 mg of furosemide).

### Example: A Capsule Including Amlodipine

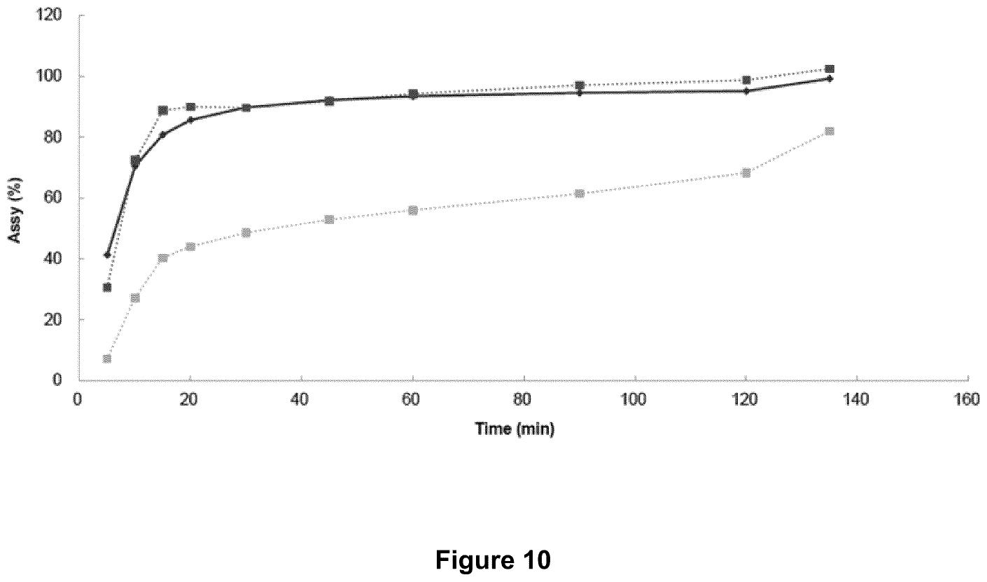

Referring briefly to FIG. 10, a chart is provided that depicts of a release profile (e.g., dissolution results) of an amlodipine pharmaceutical composition and at least second pharmaceutical composition at a pH of 6.8 using various drug delivery system (e.g., capsules and/or tablet). More particularly, the yellow line with boxes (depicted nearest the X-axis of the chart) provides a first release profile of the amlodipine pharmaceutical composition using a conventional capsule without one or more dividers 220 of the present disclosure, the dark red line with diamonds illustrates a target release profile of the amlodipine pharmaceutical composition (e.g., a tablet including amlodipine), and the bright red line with squares (depicted furthest from the X-axis of the chart) provides a second release profile of the amlodipine pharmaceutical composition using capsule 200 with one or more dividers 220 of the present disclosure. As shown, the second release profile for the capsule of the present disclosure is equivalent to the target release profile. However, the first release profile of the amlodipine pharmaceutical composition in which the amlodipine is not physically separated from the at least second pharmaceutical composition illustrates that, without the separation provided by dividers of the present disclosure, the amlodipine pharmaceutical composition in the conventional drug delivery system interact with each other, and cause the release profile of the amlodipine to be=different from the target release profile.

Referring briefly to FIG. 11, an exemplary method for manufacturing multicompartment capsule 200 of the present disclosure is provided. First hollow body 210-1 is positioned to allow first pharmaceutical composition 142-1 to be inserted into a first unique interior volume of the capsule. First divider 220-1 is then inserted into the first hollow body. In some embodiments, layer of material 600 on a surface of the first divider ensures the first divider remains at a desired position within the first hollow body 210-1. Second pharmaceutical composition 142-2 is inserted into a second unique interior volume of the capsule. Second divider 220-2 is then inserted into the first hollow body. Third pharmaceutical composition 142-3 is inserted into a second unique interior volume of the capsule. Finally, second hollow body 210-2 is replaced over the first hollow body and coupled to the first hollow body. In some embodiments, the first and second hollow bodies are sealed in place by known means by adhesive, a sealing strip, welding (e.g., thermal welding, sonic welding, etc.) or the like.

In various embodiments, the invention provides a method of devising a single unit dosage formulation of a pharmaceutical composition for a selected subject to prevent, treat, or ameliorate a selected condition of the subject (e.g., a hypertension condition, a coronary artery disease condition, a pericarditis condition, a systolic heart failure condition (e.g., a heart failure with reduced ejection fraction (HFrEF) condition)), a combination thereof, etc.). The method includes determining an appropriate dosages of two or more agents of use to prevent, treat, or ameliorate the condition taking into consideration the release rate of a first pharmaceutical composition in a first unique interior volume, and the size of this volume; taking into consideration the release rate of a second pharmaceutical composition in a second unique interior volume, and the size of this volume; and determining an effective dosage of each of the first and second pharmaceutical compositions.

In an exemplary embodiment, the invention provides a method of preventing, treating or ameliorating a condition in a patient by administering to a subject in need thereof a capsule of the invention containing at least a first and a second pharmaceutical composition known to be of use in preventing, treating or ameliorating in an appropriate dosage of each pharmaceutical composition for preventing, treating or ameliorating a condition in the subject to whom the capsule is administered.

In some embodiments, the invention provides a method of fulling an order for a capsule of the present disclosure. In some embodiments, the method includes communication a provision of the capsule to a subject (e.g., client of computer system 100 of FIG. 1). In some embodiments, the method includes communicating an over the counter (OTC) drug facts label for each respective pharmaceutical compositions in a plurality of pharmaceutical compositions of the capsule (e.g., first OTC drug facts label for first pharmaceutical composition 142-1 of capsule 200 of FIG. 9, second OTC drug facts label for second pharmaceutical composition 142-2 of capsule 200 of FIG. 9, third OTC drug facts label for third pharmaceutical composition 142-3 of capsule 200 of FIG. 9, etc.).

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that includes a computer program mechanism embedded in a non-transitory computer-readable storage medium. For instance, the computer program product could contain instructions for operating the user interfaces disclosed herein and described with respect to the Figures. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed is:

1. A capsule comprising:
a first hollow body and a second hollow body configured to couple together, thereby collectively forming an interior volume of the capsule, wherein each hollow body comprises a closed end portion;
a first divider, wherein the first divider comprises:
a base,
a sidewall protruding from a first circumferential edge portion of the base, the sidewall comprising an inner surface, an outer surface, and a second circumferential edge portion, the second circumferential edge portion configured to continuously engage a unique circumferential surface of the capsule, thereby securing the first divider within the interior volume of the capsule and forming at least two unique interior volumes of the interior volume based on a position of the first divider and the unique circumferential surface of the capsule; and
a plurality of pharmaceutical compositions, wherein each respective pharmaceutical composition in the plurality of pharmaceutical compositions occupies a unique interior volume of the at least two interior volumes.

2. The capsule of claim 1, wherein the first divider has a frustum shape.

3. The capsule of claim 1, further comprising a line of symmetry about a longitudinal axis of the capsule.

4. The capsule of claim 1, wherein the interior volume of the capsule is in a range of from about 0.1 milliliters (mL) to about 1.5 mL.

5. The capsule of claim 1, wherein the second circumferential edge portion deforms radially.

6. The capsule of claim 1, wherein the inner surface and/or the outer surface of the sidewall is generally concave or generally convex.

7. The capsule of claim 1, wherein the base further comprises an upper surface and a lower surface, and wherein the upper surface and/or the lower surface of the base is generally concave or generally convex.

8. The capsule of claim 7, further comprising a layer of a material disposed on a portion of the upper surface, the material configured to prevent the upper surface from adhering to a different surface or face other than the layer of the material.

9. The capsule of claim 8, wherein the material comprises a coefficient of friction in a range of from 0.1 to 0.8.

10. The capsule of claim 1, wherein the base further comprises an upper surface and a lower surface, and wherein the upper surface and the lower surface of the base are parallel or substantially parallel.

11. The capsule of claim 1, wherein the second circumferential edge portion comprises a chamfer or rounded edge.

12. The capsule of claim 11, wherein an outer diameter of the second circumferential edge portion exclusively engages the unique circumferential surface of the capsule.

13. The capsule of claim 1, wherein the base of each divider further comprises a third circumferential edge portion.

14. The capsule of claim 13, wherein the second circumferential edge portion comprises a first diameter and the third circumferential edge portion comprises a second diameter different from the first diameter.

15. The capsule of claim 14, wherein the first hollow body or the second hollow body comprises a third diameter greater than or equal to the second diameter.

16. The capsule of claim 15, wherein the third diameter is less than or equal to the first diameter.

17. The capsule of claim 13, wherein the third circumferential edge portion comprises a chamfer or rounded edge.

18. The capsule of claim 1, wherein a draft angle of the sidewall is greater than zero.

19. The capsule of claim 18, wherein the draft angle is in a range of from about 0.1 degrees (°) to about 5°.

20. The capsule of claim 1, wherein a characteristic length of the sidewall is in a range of from about 0.5 millimeters (mm) to about 20 mm.

21. The capsule of claim 1, wherein the first hollow body, the second hollow body, the first divider, or a combination thereof is transparent, translucent, or opaque.

22. The capsule of claim 1, wherein the first divider is a monolithic body.

23. The capsule of claim 1, wherein the plurality of pharmaceutical compositions comprises one or more liquid formulations, one or more solid formulations, one or more powdered formulations, one or more granule formulations, one or more gel formulations, or a combination thereof.

24. The capsule of claim 1, wherein the first divider is defined by a first orientation, and wherein the capsule further comprises a second divider defined by a second orientation.

25. The capsule of claim 24, wherein the first orientation is different from the second orientation.

26. The capsule of claim 1, wherein the first divider, the first hollow body, the second hollow body, or a combination thereof comprises a water soluble polymer, an erodible polymer, a degradable polymer, a swellable polymer, or a combination thereof.
